(19) United States
(12) Patent Application Publication
(10) Pub. No.: US 2022/0135564 A1
(43) Pub. Date: May 5, 2022

(54) DEUTERATED MITRAGYNINE ANALOGS AS SAFER OPIOID MODULATORS IN THE MITRAGYNINE CLASS

(71) Applicants: The Trustees of Columbia University in the City of New York, New York, NY (US); The Research Foundation for Mental Hygiene, Inc., Menands, NY (US); Sloan-Kettering Institute for Cancer Research, New York, NY (US)

(72) Inventors: Andrew C. Kruegel, Secaucus, NJ (US); Dalibor Sames, New York, NY (US); Jonathan A. Javitch, Dobbs Ferry, NY (US); Susruta Majumdar, St. Louis, MO (US)

(73) Assignees: The Trustees of Columbia University in the City of New York, New York, NY (US); The Research Foundation for Mental Hygiene, Inc., Menands, NY (US); Sloan-Kettering Institute for Cancer Research, New York, NY (US)

(21) Appl. No.: 17/427,502

(22) PCT Filed: Jan. 30, 2020
(86) PCT No.: PCT/US2020/015898

§ 371 Date: Jul. 30, 2021

Related U.S. Application Data
(60) Provisional application No. 62/800,369, filed on Feb. 1, 2019.

(51) Int. Cl. C07D 471/14

(52) U.S. Cl. CPC C07D 471/14; A61K 31/485

(57) ABSTRACT

The present invention provides a compound having the structure:

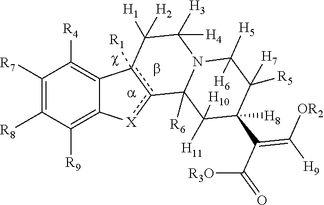

or a pharmaceutically acceptable salt or ester thereof, and methods of using the compound to treat pain, depressive disorders, mood disorders, anxiety disorders, opioid use disorder, and opioid withdrawal symptoms.

This application claims priority of U.S. Provisional Application No. 62/800,369, filed Feb. 1, 2019, the contents of which are hereby incorporated by reference.

Throughout this application, certain publications are referenced in parentheses. Full citations for these publications may be found immediately preceding the claims. The disclosures of these publications in their entireties are hereby incorporated by reference into this application in order to describe more fully the state of the art to which this invention relates.

### BACKGROUND OF THE INVENTION

The opioid receptors, and in particular, the mu-opioid receptor (MOR), are among the longest and most intensely studied molecular signaling systems in the central nervous system (Pasternak, G. W. et al. 2013). Likewise, the prototypical small molecule agonist of these receptors, morphine, has been used by humans as an important analgesic and recreational euphoriant since ancient times. Indeed, MOR agonists, including not only morphine itself but also a vast number of synthetic and semi-synthetic opioids, remain the gold standard of pain therapy. Unfortunately, acute MOR activation is also associated with serious side effects, including respiratory depression, constipation, sedation, nausea, and itching (Pasternak, G. W. et al. 2013; Inturrissi, C. E. 2002). At sufficiently high doses, the evoked respiratory depression can be severe enough to cause death. Further, the pronounced euphoria produced by MOR agonists makes them major drugs of abuse. These properties have made overdose from prescription opioid analgesics a leading cause of accidental death in the United States, killing more than 18,000 people in 2014 (NIDA 2015). Another shortcoming of MOR agonists is the rapid development of tolerance to their analgesic effects. Thus, continuing escalation of a dose is required to maintain an equivalent level of pain management. Similarly, when they are abused, tolerance to the euphoric effects of opioids is also rapidly developed. Thus, in either case, chronic use often results in severe physical dependence on MOR agonists due to cellular- and circuit-level adaptations to continuous receptor stimulation. Accordingly, much effort has been dedicated to the development of new MOR agonists retaining potent analgesic effects, while mitigating or eliminating the deleterious side effects of the agents currently in use (Pasternak, G. W. et al. 2013; Inturrissi, C. E. 2002; Pasternak, G. W. et al. 2010; Grinnell, S. G. et al. 2014; Largent-Milnes, T. et al. 2010; Stevenson, G. W. et al. 2015).

Historically, MOR agonists have also been applied in the treatment of mood disorders, notably including major depressive disorder (MDD). Indeed, until the mid-20th century, low doses of opium itself were used to treat depression, and the so-called “opium cure” was purportedly quite effective (Kraepelin, E. 1905). With the advent of tricyclic antidepressants (TCAs) in the 1950s however, the psychiatric use of opioids rapidly fell out of favor and has been largely dormant since likely due to negative medical and societal perceptions stemming from their abuse potential. However, there have been scattered clinical reports (both case studies and small controlled trials) since the 1970s indicating the effectiveness of MOR agonists in treating depression. The endogenous opioid peptide β-endorphin, as well as a number of small molecules, have all been reported to rapidly and robustly improve the symptoms of MDD and/or anxiety disorders in the clinical setting, even in treatment-resistant patients (Gerner, R. H. et al. 1980; Stoll, A. L. 1999; Dean, A. J. et al. 2004; Shapira, N. A. et al. 2001; Shapira, N. A. et al. 1997; Emrich, H. M. et al. 1982; Karp, J. F. et al. 2014; Bodkin, J. A. et al. 1995). These results have been recapitulated in rodent models, where a variety of MOR agonists have shown antidepressant effects (Besson, A. et al. 1996; Rojas-Corrales, M. O. et al. 2002; Fichna, J. et al. 2007; Rojas-Corrales, M. O. et al. 1998). Most recently, it was found that the atypical antidepressant tianeptine, which has been used clinically for several decades and extensively studied in rodents and other mammalian species, is a MOR agonist, suggesting that this agent exerts its antidepressant effects via direct MOR activation (Gassaway, M. M. et al. 2014; Samuels, B. A. et al. 2017). Mitragyna speciosa, commonly known as kratom, is a psychoactive plant native to Southeast Asia, where its leaves are used by humans for their mild stimulant effects and medicinal properties, including for the treatment of pain and opioid addiction. Mitragynine is the predominant psychoactive alkaloid found in kratom and is believed to be an important contributor to the plant's medicinal properties. Several other alkaloids, namely speciogynine, paynantheine, and speciociliatine, are also present in significant quantities in kratom and may contribute to the psychoactive and therapeutic properties of the plant (Kruegel, A. C. and Grundman, O. 2018).

Mitragynine is a partial agonist of the MOR and has analgesic and antidepressant properties in animal models (Kruegel, A. C. and Grundman, O. 2018; Kruegel, A. C. et al. 2016). It was recently discovered that mitragynine is metabolized in vivo to 7-hydroxymitragynine (7-OH), a much more potent MOR agonist and analgesic (Kruegel, A. C. et al. 2019). Additionally, data has been collected demonstrating that this metabolite is an important contributor to the analgesic and other opioid-mediated effects of mitragynine in vivo in mice.

### SUMMARY OF THE INVENTION

The present invention provides a composition which comprises a carrier and a compound having the structure:

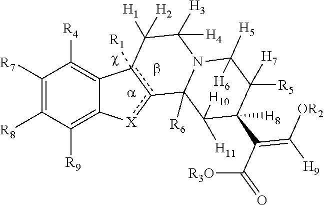

wherein
X is N or NH;
R₁ is —OH, —O-alkyl, —O—C(O)(alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O)(alkyl), —CO₂H, —CO₂-(alkyl), —NH(CO)-alkyl, —NH(CO)NH-alkyl, —NH(CO)-aryl, or —NH(CO)NH-aryl;
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester of the compound.

The present invention also provides a pharmaceutical composition which comprises a pharmaceutically acceptable carrier and a compound having

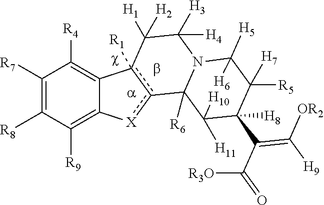

the structure:
wherein
X is N or NH; R₁ is —OH, —O-alkyl, —O—C(O)(alkyl), or is absent;
R₂ is —H or -alkyl;
R₃ is —H or -alkyl;
R₄ is —H, —F, —Cl, —Br, —I, -alkyl, -alkenyl, -alkynyl, —CN, CF₃, —NO₂, —OH, —NH₂, —SH, —C(O)NH₂, —C(O)NH(alkyl), C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —O-alkyl, —S-alkyl, —O-aryl, —S-aryl, —O-heteroaryl, —S— heteroaryl, -aryl, -heteroaryl, —O—C(O)(alkyl), —CO₂H, —CO₂-(alkyl), —NH(CO)-alkyl, —NH(CO)NH— alkyl, —NH(CO)-aryl, or —NH(CO)NH-aryl;
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester of the compound.

### BRIEF DESCRIPTION OF THE FIGURES

FIG. 1: 3-Dehydromitragynine (DHM) hydrochloride dramatically impairs motor coordination in male mice (C57BL/6J) compared to vehicle treatment, as evidenced by decreased latency to fall in the rotarod test. Data points represent mean±SEM; n=10 per treatment.

FIG. 2A: 3-Dehydromitragynine (DHM) is lethally toxic to mice. Groups of mice (n=6 per dose) were treated subcutaneously (s.c.) with different doses of DHM and tested for lethality 24 h after drug administration. The experiment was performed in the 129Sv6 strain. DHM had an LD₅₀ of 48.4 (71.27-342.3) mg/kg in 129Sv6 mice. Numbers in parentheses are 95% confidence intervals.

FIG. 2B: 3-Dehydromitragynine (DHM) is lethally toxic to mice. Groups of mice (n=6 per dose) were treated subcutaneously (s.c.) with different doses of DHM and tested for lethality 24 h after drug administration. The experiment was performed in the CD-1 strain. DHM had an LD₅₀ of 74 (48.08-120.5) mg/kg in CD-1 mice. Numbers in parentheses are 95% confidence intervals.

FIG. 3A: Deuteration attenuates formation of toxic metabolite 3-dehydromitragynine (DHM) in human liver microsomes (HLMs). Mitragynine and 3-deuteromitragynine (3-DM) were incubated with HLMs and concentrations of DHM were determined at the indicated time points. Deuteration, as in 3-DM, greatly attenuated the concentration of toxic metabolite DHM formed in HLMs compared to mitragynine. Data points represent mean±SEM of two incubations.

FIG. 3B: Deuteration does not attenuate formation of 7-hydroxy active metabolites in human liver microsomes (HLMs). Mitragynine and 3-deuteromitragynine (3-DM) were incubated with HLMs and concentrations of 7-hydroxymitragynine (7-OH, in the case of mitragynine) or 3-deutero-7-hydroxymitragynine (3-d-7-OH, in the case of 3-DM) were determined at the indicated time points. Deuteration, as in 3-DM, had little effect on formation of the corresponding active metabolite 3-d-7-OH. Data points represent mean±SEM of two incubations.

FIG. 4A: Deuteration attenuates mitragynine decomposition in mouse brain homogenate (MBH). Mitragynine and 3-deuteromitragynine (3-DM) were incubated in MBH and disappearance of parent compounds was monitored at the indicated time points. Deuteration, as in 3-DM, attenuated the decomposition of the parent compound in MBH compared to mitragynine. Data points represent mean±SEM of two incubations.

FIG. 4B: Deuteration attenuates formation of toxic metabolite 3-dehydromitragynine (DHM) in mouse brain homogenate (MBH). Mitragynine and 3-deuteromitragynine (3-DM) were incubated in MBH and formation of DHM was monitored at the indicated time points. Deuteration, as in 3-DM, attenuated formation of DHM in MBH compared to mitragynine.

FIG. 5A: Deuteration attenuates formation of toxic metabolite 3-dehydromitragynine (DHM) in mice. Male mice (129S1) were treated with mitragynine or 3-deutromitragynine (3-DM) (10 mg/kg, s.c.) and brain concentrations of DHM (as the hydrochloride) were determined at 15 minutes. Deuteration, as in 3-DM, greatly attenuated the concentration of toxic metabolite DHM found in the brain compared to mitragynine. Data bars represent mean±SEM; n=4 for mitragynine; n=1 for 3-DM.

FIG. 5B: Deuteration does not attenuate formation of 7-hydroxy active metabolites in mice. Male mice (129S1) were treated with mitragynine or 3-deutromitragynine (3-DM) (10 mg/kg, s.c.) and brain concentrations of 7-hydroxymitragynine (7-OH, in the case of mitragynine) or 3-deutero-7-hydroxymitragynine (3-d-7-OH, in the case of 3-DM) were determined at 15 minutes. Deuteration, as in 3-DM, had little effect on formation of the corresponding active metabolite 3-d-7-OH. Data bars represent mean±SEM; n=4 for mitragynine; n=1 for 3-DM.

FIG. 6A: Deuteration does not attenuate 7-OH decomposition in simulated gastric fluid (SGF). 7-Hydroxymitragynine (7-OH) and 3-deutero-7-hydroxymitragynine (3-d-7-OH) were dissolved in deuterated SGF at a concentration of 1.3 mg/mL and incubated at room temperature. Disappearance of parent compounds was monitored directly by NMR spectroscopy at the following time points: 35, 65, 125, 245, 365, and 1440 minutes. Deuteration, as in 3-d-7-OH, did not slow decomposition of the parent compound compared to 7-OH.

FIG. 6B: Deuteration attenuates formation of toxic metabolite 3-dehydromitragynine (DHM) in simulated gastric fluid (SGF). 7-Hydroxymitragynine (7-OH) and 3-deutero-7-hydroxymitragynine (3-d-7-OH) were dissolved in deuterated SGF at a concentration of 1.3 mg/mL and incubated at room temperature. Formation of DHM was monitored directly by NMR spectroscopy at the following time points: 35, 65, 125, 245, 365, and 1440 minutes. Deuteration, as in 3-d-7-OH, greatly attenuated the formation of toxic metabolite DHM. The concentration of DHM formed from 3-d-7-OH was below the lower limit of quantitation of -0.1 mM at the 35-, 65-, and 125-minute time points.

FIG. 7A: Deuteration attenuates formation of toxic metabolite 3-dehydromitragynine (DHM) in mice. Male mice (C57BL/6) were treated with mitragynine or 3-deutromitragynine (3-DM) (10 mg/kg, s.c.) and plasma concentrations of DHM were determined at the indicated time points. Deuteration, as in 3-DM, greatly attenuated the concentration of toxic metabolite DHM found in the plasma compared to that found with mitragynine. Two-way ANOVA: F_{1,42}=138.4, p<0.0001. All data points represent mean±SEM; n=4 per time point, per treatment.

FIG. 7B: Deuteration does not attenuate formation of 7-OH active metabolites in mice. Male mice (C57BL/6) were treated with mitragynine or 3-deutromitragynine (3-DM) (10 mg/kg, s.c.) and plasma concentrations of 7-hydroxymitragynine (7-OH, in the case of mitragynine) or 3-deutero-7-hydroxymitragynine (3-d-7-OH, in the case of 3-DM) were determined at the indicated time points. Deuteration, as in 3-DM, had no effect on the concentration of active metabolite 3-d-7-OH found in the plasma compared to the 7-OH found with mitragynine. Two-way ANOVA: F_{1,42}=0.0003117, ns. All data points represent mean±SEM; n=4 per time point, per treatment.

FIG. 7C: Deuteration attenuates formation of toxic metabolite 3-dehydromitragynine (DHM) in mice. Male mice (C57BL/6) were treated with mitragynine or 3-deutromitragynine (3-DM) (10 mg/kg, s.c.) and brain concentrations of DHM were determined at the indicated time points. Deuteration, as in 3-DM, greatly attenuated the concentration of toxic metabolite DHM found in the brain compared to that found with mitragynine. Two-way ANOVA: F_{1,42}=32.44, p<0.0001. All data points represent mean±SEM; n=4 per time point, per treatment.

FIG. 7D: Deuteration does not attenuate formation of 7-OH active metabolites in mice. Male mice (C57BL/6) were treated with mitragynine or 3-deutromitragynine (3-DM) (10 mg/kg, s.c.) and brain concentrations of 7-hydroxymitragynine (7-OH, in the case of mitragynine) or 3-deutero-7-hydroxymitragynine (3-d-7-OH, in the case of 3-DM) were determined at the indicated time points. Deuteration, as in 3-DM, had no effect on the concentration of active metabolite 3-d-7-OH found in the brain compared to the 7-OH found with mitragynine. Two-way ANOVA: F₁,42=0.8888, ns. All data points represent mean±SEM; n=4 per time point, per treatment.

FIG. 8A: Deuteration attenuates formation of metabolite M1 in mouse liver S9 fraction (MS9). Mitragynine and 3-deuteromitragynine (3-DM) were incubated with MS9 and M1 was quantified by mass spectrometric peak area at the indicated time points. Deuteration, as in 3-DM, greatly attenuated the formation of metabolite M1 in the presence of MS9 compared to mitragynine. Data points represent mean±SEM of two incubations.

FIG. 8B: Deuteration attenuates formation of metabolite M4 in mouse liver S9 fraction (MS9). Mitragynine and 3-deuteromitragynine (3-DM) were incubated with MS9 and M4 was quantified by mass spectrometric peak area at the indicated time points. Deuteration, as in 3-DM, greatly attenuated the formation of metabolite M4 in the presence of MS9 compared to mitragynine. Data points represent mean±SEM of two incubations.

FIG. 8C: Deuteration attenuates formation of metabolite M6 in mouse liver S9 fraction (MS9). Mitragynine and 3-deuteromitragynine (3-DM) were incubated with MS9 and M6 was quantified by mass spectrometric peak area at the indicated time points. Deuteration, as in 3-DM, greatly attenuated the formation of metabolite M6 in the presence of MS9 compared to mitragynine. Data points represent mean±SEM of two incubations.

FIG. 9: Mitragynine and 3-deuteromitragynine (3-DM) exhibited dose-dependent analgesic effects in the rat tail-flick assay. Groups of rats were treated with vehicle or ascending doses of test compounds and analgesic activity was assessed in the tail-flick assay using a 50° C. hot-water bath 30 minutes after drug administration. All data points represent mean±SEM; n=8 per treatment.

FIG. 10A: Deuteration attenuates 7-hydroxymitragynine (7-OH) decomposition in dog plasma (DP). 7-OH and 3-deutero-7-hydroxymitragynine (3-d-7-OH) were incubated in DP and disappearance of parent compounds was monitored at the indicated time points. Deuteration, as in 3-d-7-OH, attenuated the decomposition of the parent compound in DP compared to 7-OH. Data points represent mean±SEM of two incubations.

FIG. 10B: Deuteration attenuates formation of toxic metabolite 3-dehydromitragynine (DHM) in dog plasma (DP). 7-OH and 3-deutero-7-hydroxymitragynine (3-d-7-OH) were incubated in DP and formation of DHM was monitored at the indicated time points. Deuteration, as in 3-d-7-OH, attenuated formation of DHM in DP compared to 7-OH. Data points represent mean±SEM of two incubations.

### DETAILED DESCRIPTION OF THE INVENTION

The present invention provides a composition which comprises a carrier and a compound having the structure:

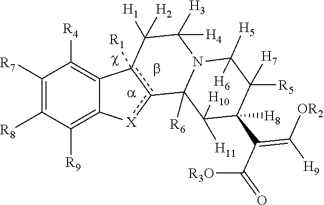

wherein
X is N or NH;
R₁ is —OH, —O-alkyl, —O—C(O)(alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site;
R₇, R₈ and R₉ are each, independently, —H, —F, —Cl, —Br, —I, -alkyl, -alkenyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O)(alkyl), —CO₂H, —CO₂-(alkyl), —NH(CO)-alkyl, —NH(CO)NH-alkyl, —NH(CO)-aryl, or
NH(CO)NH-aryl;
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R is present,
      or a pharmaceutically acceptable salt or ester of the compound.

The present invention provides a composition which comprises a carrier and a compound having the structure:

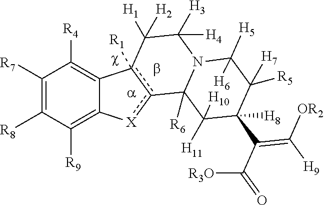

wherein
X is N or NH;
R₁ is —OH, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl or alkenyl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O) (alkyl), —CO₂H, or —CO₂-(alkyl);
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester of the compound.

In some embodiments, the composition wherein the compound has the structure:

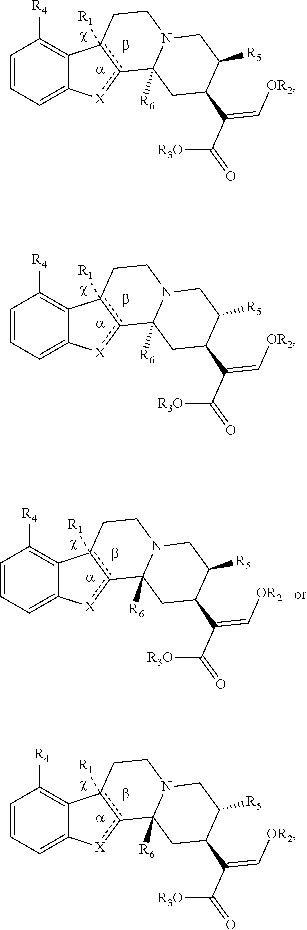

or a pharmaceutically acceptable salt or ester thereof.

In some embodiments, the composition wherein the compound has the structure:

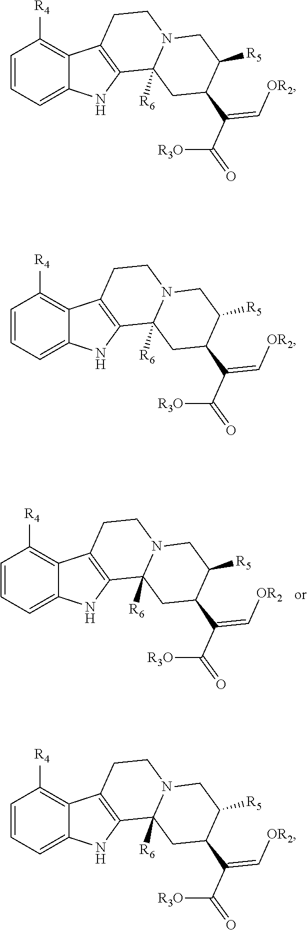

or a pharmaceutically acceptable salt or ester thereof.

In some embodiments, the composition wherein the compound has the structure:

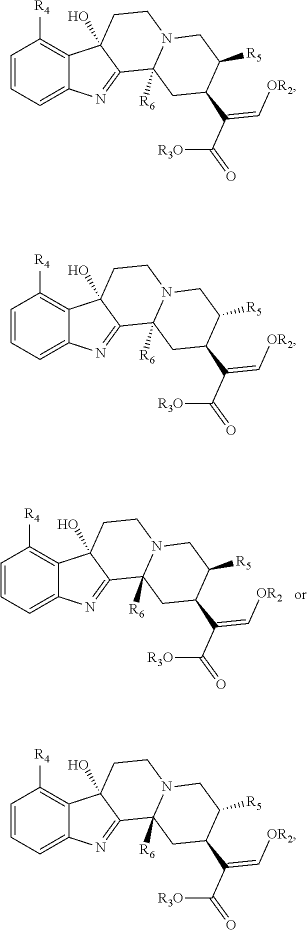

or a pharmaceutically acceptable salt or ester thereof.

In some embodiments, the composition wherein R₂ and R₃ are each methyl.

In some embodiments, the composition wherein R₄ is methoxy.

In some embodiments, the composition wherein R₅ is ethyl or vinyl.

In some embodiments, the composition wherein one or more or all of H1-H11 are deuterium-enriched.

In some embodiments, the composition wherein R₆ is a deuterium-enriched —H site.

In some embodiments, the composition wherein at least one of R₇, R₈ or R₉ is a deuterium-enriched —H site.

In some embodiments, the composition wherein H₁₀ and/or H₁₁ is a deuterium-enriched —H site.

In some embodiments, the composition wherein R₆ is methyl.

In some embodiments, the composition wherein the compound has the structure:

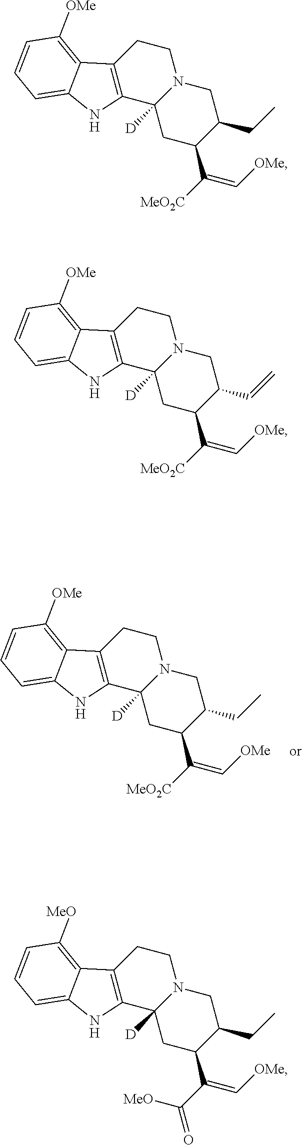

wherein D represents a deuterium-enriched —H site or a pharmaceutically acceptable salt or ester thereof.

In some embodiments, the composition wherein the compound has the structure:

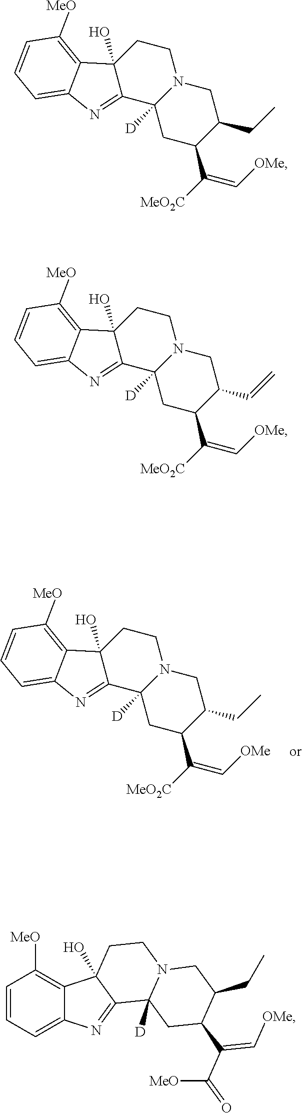

wherein D represents a deuterium-enriched —H site or a pharmaceutically acceptable salt or ester thereof.

In some embodiments, the composition wherein the compound has the structure:

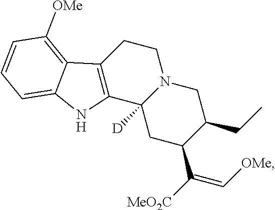

wherein D represents a deuterium-enriched —H site or a pharmaceutically acceptable salt or ester thereof.

In some embodiments, the composition wherein the compound has the structure:

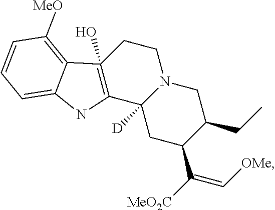

wherein D represents a deuterium-enriched site or a pharmaceutically acceptable salt or ester thereof.

In some embodiments, the composition wherein the compound has the structure:

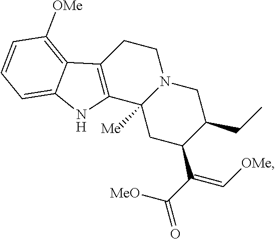 [SMILES: CC[C@@H]1CN2CCc3c([nH]c4cccc(OC)c34)[C@]2(C)C[C@@H]1/C(=C\OC)C(=O)OC]

or a pharmaceutically acceptable salt or ester thereof.

In some embodiments, the composition wherein the compound has the structure:

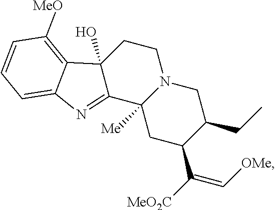 [SMILES: CC[C@@H]1CN2CC[C@@]3(O)C(=Nc4cccc(OC)c43)[C@]2(C)C[C@@H]1/C(=C\OC)C(=O)OC]

or a pharmaceutically acceptable salt or ester thereof.

In some embodiments, a pharmaceutical composition comprising the composition of the present invention wherein the carrier is a pharmaceutically acceptable carrier.

In some embodiments, a pharmaceutical composition comprising (i) the composition of the present invention wherein the carrier is a pharmaceutically acceptable carrier; and (ii) an NMDA receptor antagonist, an NMDA receptor partial agonist, a neurokinin 1 receptor antagonist, a neurokinin 2 receptor antagonist, a neurokinin 3 receptor antagonist, a DOR agonist, naloxone, methylnaltrexone, a selective serotonin reuptake inhibitor or a serotonin-norepinephrine reuptake inhibitor.

The present invention also provides a pharmaceutical composition which comprises a pharmaceutically acceptable carrier and a compound having the structure:

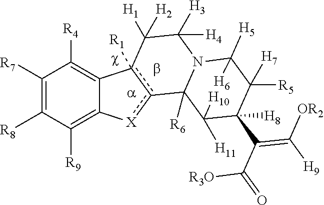

wherein
X is N or NH;
R₁ is —OH, —O-alkyl, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, CF₃, —NO₂, —OH, —NH₂, —SH, —C(O)NH₂, —C(O)NH(alkyl), C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O) (alkyl), —CO₂H, —CO₂-(alkyl), —NH(CO)-alkyl, —NH(CO)NH-alkyl, —NH(CO)-aryl, or NH(CO)NH-aryl;
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester thereof.

The present invention also provides a pharmaceutical composition which comprises a pharmaceutically acceptable carrier and a compound having the structure:

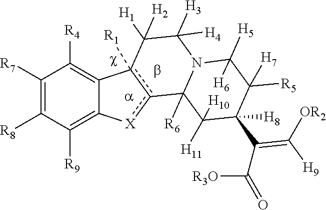

wherein
X is N or NH;
R₁ is —OH, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl or alkenyl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O)(alkyl), —CO₂H, or —CO₂-(alkyl);
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester thereof.

In some embodiments, the composition wherein R₆ is a deuterium-enriched —H site and the level of deuterium at the deuterium-enriched —H site of the compound is 0.02% to 100%.

In some embodiments, the composition wherein R₆ is a deuterium-enriched —H site and the level of deuterium at the deuterium-enriched —H site of the compound is 20%-100%, 50%-100%, 70%-100%, 90%-100%, 97%-100%, or 99%-100%.

In some embodiments, the composition of wherein R₆ is a deuterium-enriched —H site and the level of deuterium at the deuterium-enriched —H site of the compound is no less than 50%, no less than 70%, no less than 90%, no less than 97% or no less than 99%.

In some embodiments, the composition wherein the compound has the structure:

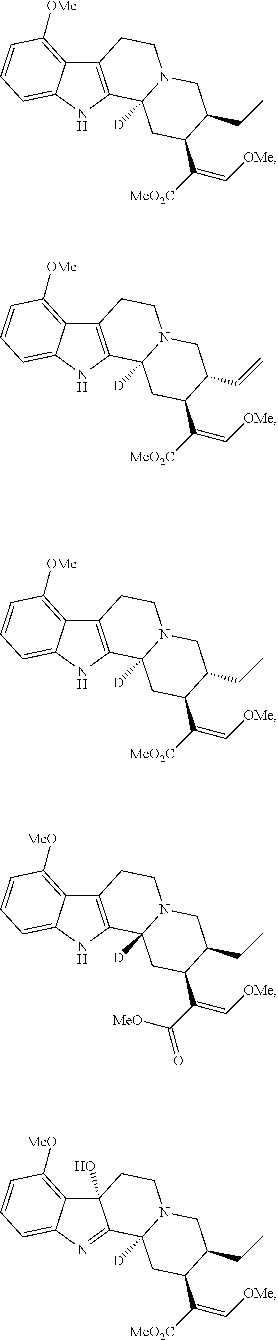

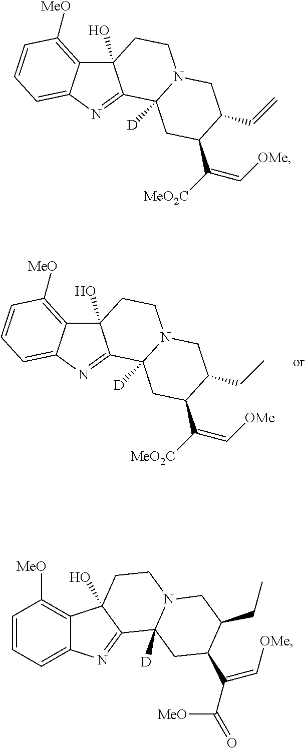

wherein D represents a deuterium-enriched —H site or a pharmaceutically acceptable salt or ester thereof.

In some embodiments, the above composition wherein the compound has the structure:

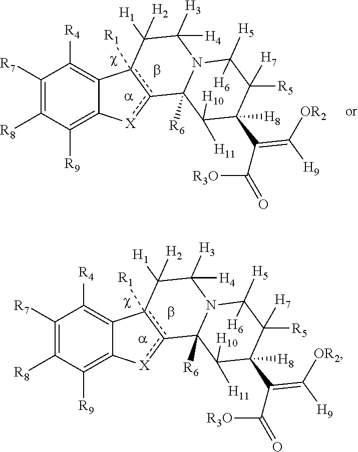

or a pharmaceutically acceptable salt or ester of the compound.

In some embodiments, the above composition wherein the compound has the structure:

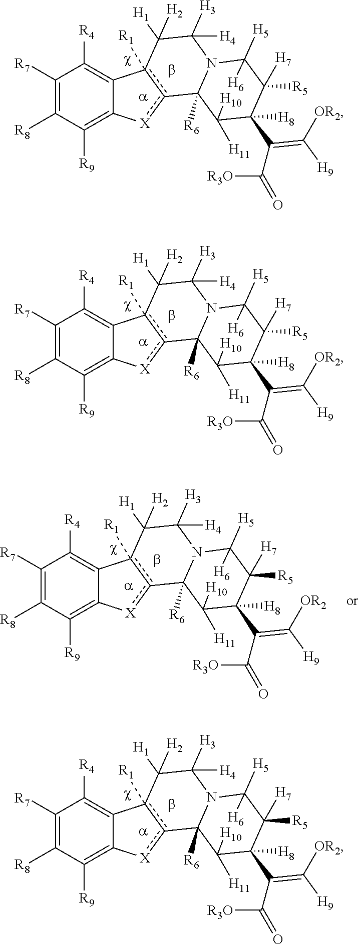

or a pharmaceutically acceptable salt or ester of the compound.

In some embodiments of any of the above composition, the compound
wherein
R₁ is —OH or is absent;
R₄ is —H, —OH or —O—C(O)(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site; and
R₇, R₈ and R₉ are each —H;
   or a pharmaceutically acceptable salt or ester thereof.

In some embodiments of any of the above composition, the compound
wherein
R₁ is —OH or is absent;
R₄ is —H, —OH or —O—C(O) (alkyl);
R₅ is alkyl or alkenyl;
R₆ is alkyl, aryl, or a deuterium-enriched —H site; and
R₇, R₈ and R₉ are each —H;
   or a pharmaceutically acceptable salt or ester thereof.

In some embodiments of any of the above composition, the compound
wherein
R₄, R₇, R₈ and R₉ are each —H;
   or a pharmaceutically acceptable salt or ester thereof.

In some embodiments, the composition wherein the compound has the structure:

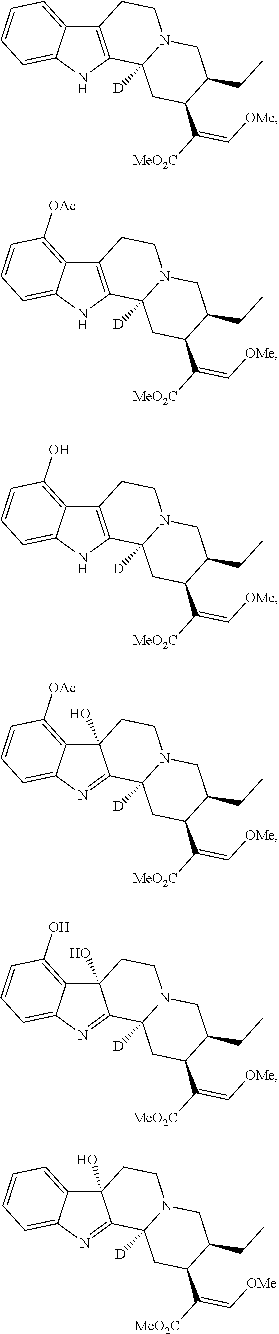

wherein D represents a deuterium-enriched —H site or a pharmaceutically acceptable salt or ester thereof.

In some embodiments of an of the above compositions, the compound
wherein
X is N or NH;
R₁ is —OH, —O-alkyl, —O—C(O) (alkyl), or is absent;
R₂ is —H or -alkyl;
R₃ is —H or -alkyl;
R₄ is —H, —OH, -alkyl or —O-alkyl;
R₅ is alkyl, alkenyl, alkyl-OH, alkyl-O-alkyl, cycloalkyl, alkyl-cycloalkyl, alkyl-aryl or alkyl-heteroaryl;
R₇, R₈ and R₉ are each, independently, —H, —F, —Cl, —Br, —I, —CN, —CF₃, —NO₂, —OH, —NH₂, —C(O)NH₂, —NH(CO)-alkyl, —NH(CO)NH— alkyl, —NH(CO)-aryl, —NH(CO)NH-aryl, —O-alkyl, —O-aryl, —O-heteroaryl, alkyl, aryl or heteroaryl;
α is a bond and is absent or present;
β is a bond and is absent or present; and
x is a bond and is absent or present,
   wherein when α is absent, β is present, χ is absent,
      X is NH and R₁ is absent, and
   wherein when α is present, β is absent, χ is present,
      X is N and R₂ is present,
         or a pharmaceutically acceptable salt or ester thereof.

In on embodiments of the above composition, the compound wherein when R₅ is ethyl, then R₈ is other than H or at least two of R₇, R₈ and R₉ are other than H, and wherein when α and χ are absent, β is present, R₂ and R₃ are each —CH₃, R₄ is —OCH₃ and each of R₇, R₈ and R₉ is —H, then R₅ is other than vinyl,

or a pharmaceutically acceptable salt or ester thereof.

In some embodiments of the composition, the compound having the structure:

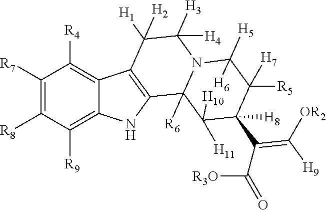

wherein
R₂ and R₃ are each, independently, —H or -alkyl;
R₄ is —H, —OH, -alkyl or —O-alkyl;
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site; and
R₇, R₈ and R₉ are each, independently, —H, —F, —Cl, —Br, —I, —CN, —CF₃, —NO₂, —OH, —NH₂, —C(O)NH₂, —NH(CO)-alkyl, —NH(CO)NH— alkyl, —NH(CO)-aryl, —NH(CO)NH-aryl, —O-alkyl, —O-aryl, —O-heteroaryl, alkyl, aryl or heteroaryl,
   or a pharmaceutically acceptable salt or ester thereof.

In on embodiments of the above composition, the compound wherein when R₅ is ethyl, then R₈ is other than H or at least two of R₇, R₈ and R₉ are other than H, and wherein when α and χ are absent, β is present, R₂ and R₃ are each —CH₃, R₄ is —OCH₃ and each of R₇, R₈ and R₉ is —H, then R₅ is other than vinyl,

or a pharmaceutically acceptable salt or ester thereof.

In some embodiments of the composition, the compound wherein R₈ is other than H or at least two of R₇, R₈ and R₉ are other than H; and R₅ is alkyl, alkenyl, alkyl-OH, alkyl-O-alkyl, cycloalkyl, alkyl-cycloalkyl, alkyl-aryl or alkyl-heteroaryl.

In some embodiments of the composition, the compound wherein R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl.

In some embodiments of the composition, the compound wherein R₇, R₈ and R₉ are each H; and R₅ is C₁-alkyl, C₃-C₁₂ alkyl, C₃-C₁₂ alkenyl, alkyl-OH, alkyl-O-alkyl, cycloalkyl, alkyl-cycloalkyl, alkyl-aryl or alkyl-heteroaryl.

In some embodiments of the composition, the compound wherein R₅ is —CH₃, —CH₂CH₂CH₃, —CH₃CH═CH₂, —CH₂OH, —CH₂-cyclopropyl, —CH₂-cyclobutyl or —CH₂CH₂-phenyl.

In some embodiments of the composition, the compound having the structure:

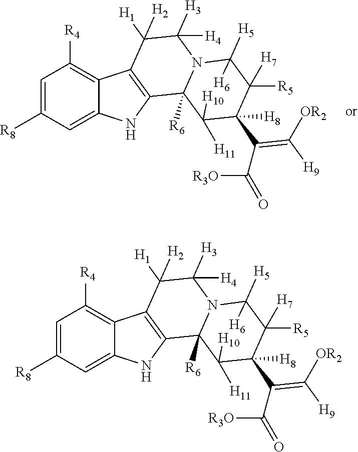

wherein
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl; and
R₈ is —Cl, —Br, —I, —CN, —CF₃, —NO₂, —OH, —CH₃, —OCH₃, —C(O)NH₂ or phenyl,
   or a pharmaceutically acceptable salt or ester thereof; or

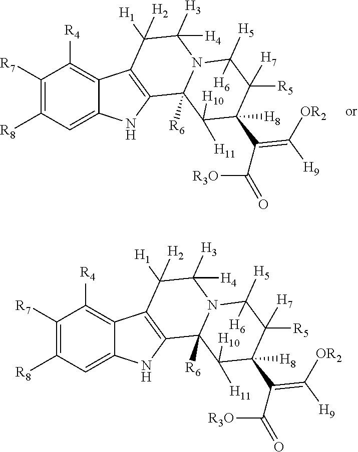

wherein
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl; and
R₇ and R₈ are each, independently, —F, —Cl, —Br, —I, —CN, —CF₃, —NO₂, —OH, —CH₃, —OCH₃, —C(O)NH₂ or phenyl,
   or a pharmaceutically acceptable salt or ester thereof; or

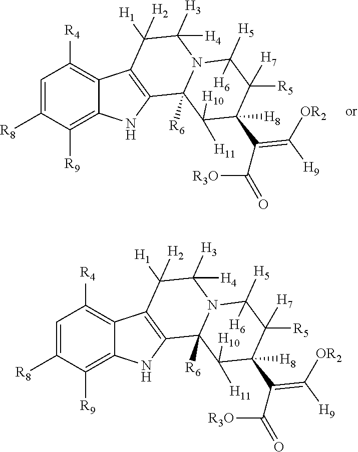

wherein
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH—CH₂, —CH₂OH, —CH₂-cyclopropyl, —CH₂-cyclobutyl or —CH₂CH₂-phenyl; and
R₈ and R₉ are each, independently, —F, —Cl, —Br, —I, —CN, —CF₃, —NO₂, —OH, —CH₃, —OCH₃, —C(O)NH₂ or phenyl, or a pharmaceutically acceptable salt or ester thereof; or

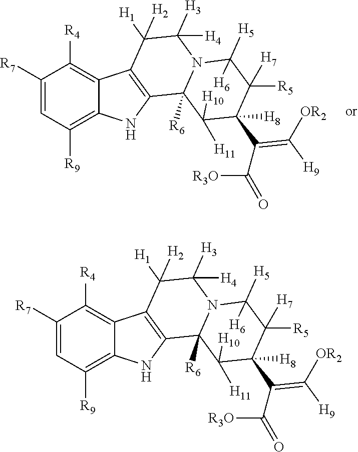

wherein
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl; and
R₇ and R₇₉ are each, independently, —F, —Cl, —Br, —I, —CN, —CF₃, —NO₂, —OH, —CH₃, —OCH₃, —C(O)NH₂ or phenyl,
   or a pharmaceutically acceptable salt or ester thereof.

In some embodiments of the composition, the compound having the structure:

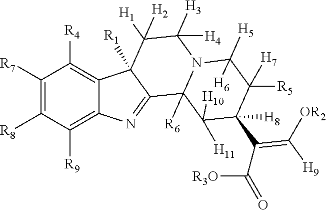

wherein
R₁ is —OH, —O-alkyl or —O(CO)-alkyl;
R₂ and R₃ are each, independently, —H or -alkyl;
R₄ is —H, —OH, -alkyl or —O-alkyl;
R₅ is alkyl, alkenyl, alkyl-OH, alkyl-O-alkyl, cycloalkyl, alkyl-cycloalkyl, alkyl-aryl or alkyl-heteroaryl; and
R₇, R₈ and R₉ are each, independently, —H, —F, —Cl, —Br, —I, —ON, —CF₃, —NO₂, —OH, —NH₂, —C(O)NH₂, —NH(CO)-alkyl, —NH(CO)NH— alkyl, —NH(CO)-aryl, —NH(CO)NH-aryl, —O-alkyl, —O-aryl, —O— heteroaryl, alkyl, aryl or heteroaryl,
   wherein when R₅ is ethyl, then R₈ is other than H or at least two of R₇, R₈ and R₉ are other than H, and
   or a pharmaceutically acceptable salt or ester thereof.

In some embodiments of the composition, the compound wherein R₉ is other than H or at least two of R₇, R₈ and R₉ are other than H; and R₅ is alkyl, alkenyl, alkyl-OH, alkyl-O-alkyl, cycloalkyl, alkyl-cycloalkyl, alkyl-aryl or alkyl-heteroaryl.

In some embodiments of the composition, the compound wherein R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl.

In some embodiments of the composition, the compound wherein R₇, R₈, and R₉ are each H; and R₅ is C₁-alkyl, C₃-C₁₂ alkyl, C₃-C₁₂ alkenyl, alkyl-OH, alkyl-O-alkyl, cycloalkyl, alkyl-cycloalkyl, alkyl-aryl or alkyl-heteroaryl.

In some embodiments of the composition, the compound wherein R₅ is —CH₃, —CH₂CH₂CH₃, —CH₃CH═CH₂, —CH₂OH, —CH₂-cyclopropyl, —CH₂-cyclobutyl or —CH₂CH₂-phenyl.

In some embodiments of the composition, the compound having the structure:

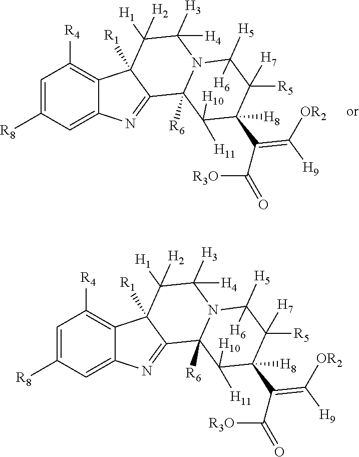

wherein
R₁ is —OH;
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl; and
R₈ is —F, —Cl, —Br, —I, —CN, —CF₃, —NO₂, —OH, —CH₃, —OCH₃, —C(O)NH₂ or phenyl, or a pharmaceutically acceptable salt or ester thereof; or

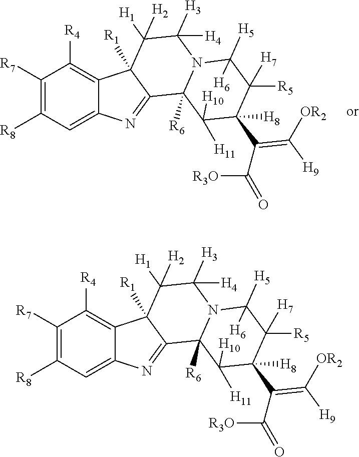

wherein
R₁ is —OH;
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂-cyclopropyl, —CH₂-cyclobutyl or —CH₂CH₂-phenyl; and
   R₇ and R₆ are each, independently, —F, —Cl, —Br, —I, —ON, —CF₃, —NO₂, —OH, —CH₃, —OCH₃, —C(O) NH₂ or phenyl,
      or a pharmaceutically acceptable salt or ester thereof; or

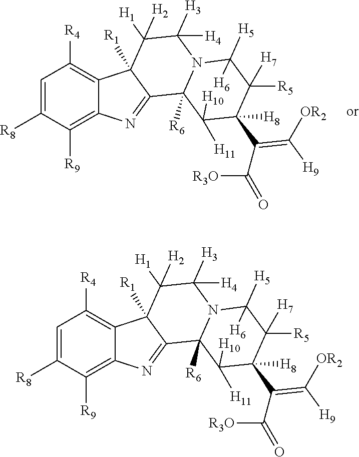

wherein
R₁ is —OH;
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂— cyclopropyl, —CH₂-cyclobutyl or —CH₂CH₂-phenyl; and
R₈ and R₉ are each, independently, —F, —Cl, —Br, —I, —CN, —CF₃, —NO₂, —OH, —CH₃, —OCH₃, —C(O)NH₂ or phenyl,
   or a pharmaceutically acceptable salt or ester thereof; or

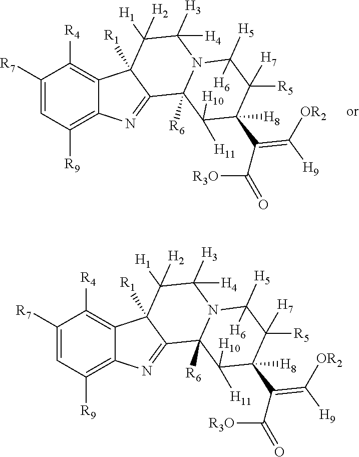

wherein
R₁ is —OH;
R₂ and R₃ are each, independently, —H or —CH₃;
R₄ is —OCH₃;
R₅ is —CH₃, —CH₂CH₃, —CH₂CH₂CH₃, —CH═CH₂, —CH₃CH═CH₂, —CH₂OH, —CH₂— cyclopropyl, —CH₂-cyclobutyl or —CH₂CH₂-phenyl; and
R₇ and R₉ are each, independently, —F, —Cl, —Br, —I, —CN, —CF₃, —NO₂, —OH, —CH₃, —OCH₃, —C(O)NH₂ or phenyl,
   or a pharmaceutically acceptable salt or ester thereof.

In some embodiments of the composition, the compound having the structure:

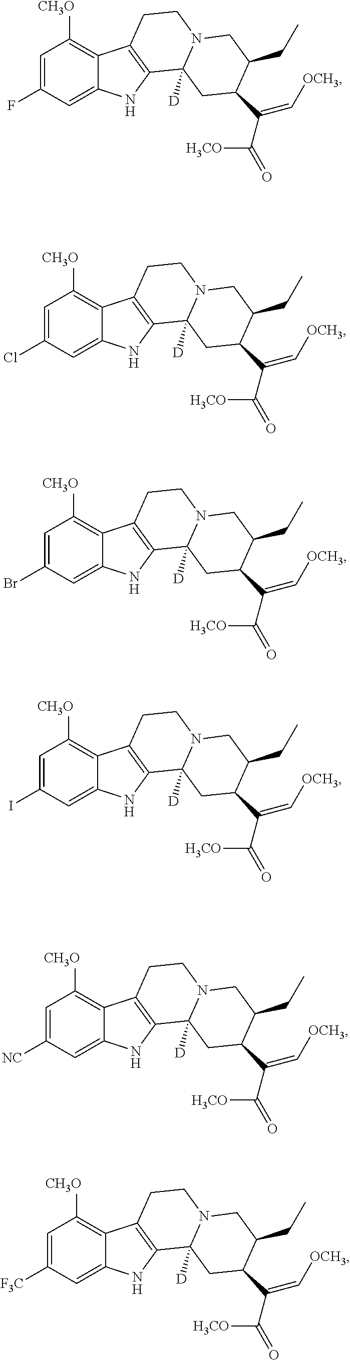

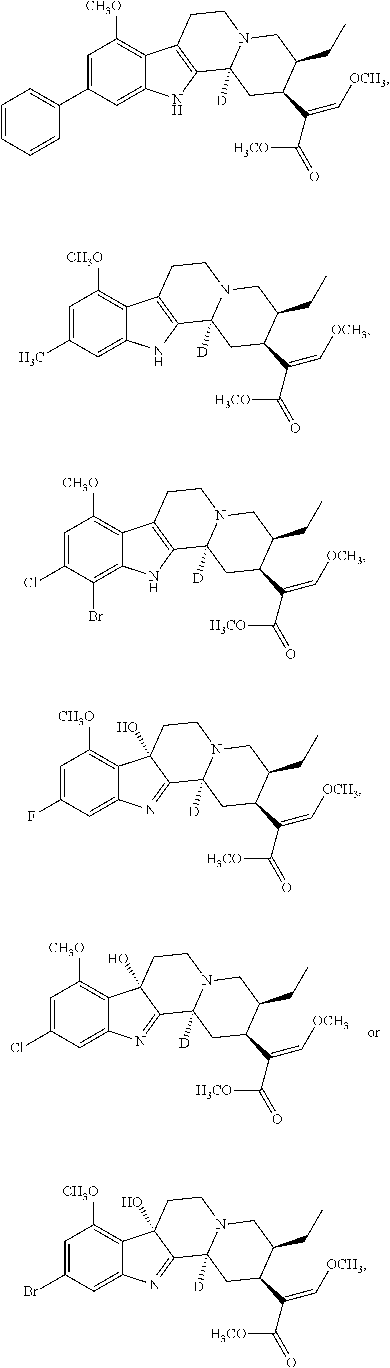

or a pharmaceutically acceptable salt or ester thereof.

The present invention provides a composition which comprises a mixture of molecules each having the structure:

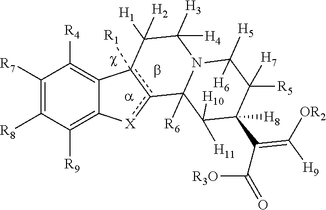

wherein
X is N or NH;
R₁ is —OH, —O-alkyl, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O)(alkyl), —CO₂H, —CO₂-(alkyl), —NH(CO)-alkyl, —NH(CO)NH— alkyl, —NH(CO)-aryl, or —NH(CO)NH-aryl;
α is a bond and is absent or present;
β is a bond and is absent or present; and
χ is a bond and is absent or present,
   wherein when α is absent, β is present, χ is absent,
   X is NH and R₁ is absent, and
   wherein when α is present, β is absent, χ is present,
   X is N and R₁ is present,
      or a pharmaceutically acceptable salt or ester of the compound,
wherein when R₆ is a deuterium-enriched —H site, the proportion of molecules having deuterium at the —R₆ position is substantially greater than 0.0156% of molecules in the composition.

The present invention provides a composition which comprises a mixture of deuterium containing and non-deuterium containing compounds having the structure:

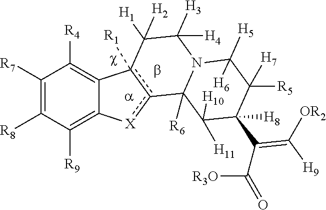

wherein
X is N or NH;
R₁ is —OH, —O-alkyl, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O)(alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O) (alkyl), —CO₂H, —CO₂-(alkyl), —NH(CO)-alkyl, —NH(CO)NH— alkyl, —NH(CO)-aryl, or —NH(CO)NH-aryl;
α is a bond and is absent or present;
β is a bond and is absent or present; and
x is a bond and is absent or present,
   wherein when α is absent, β is present, χ is absent,
   X is NH and R₁ is absent, and
   wherein when α is present, β is absent, χ is present,
   X is N and Riis present,
      or a pharmaceutically acceptable salt or ester of the compound,
wherein the proportion of molecules of the compound having deuterium at the —R₆ position is substantially greater than 0.0156% of molecules in the composition.

In some embodiments of any of the above composition, wherein the proportion of molecules of the compound having deuterium at the —R₆ position is greater than 99% of molecules in the composition.

In some embodiments of any of the above composition, wherein the proportion of molecules of the compound having deuterium at the —R₆ position is greater than 95% of molecules in the composition.

In some embodiments of any of the above composition, wherein the proportion of molecules of the compound having deuterium at the —R₆ position is greater than 90° of molecules in the composition.

In some embodiments of any of the above composition, wherein the proportion of molecules of the compound having deuterium at the —R₆ position is greater than 80% of molecules in the composition.

In some embodiments of any of the above composition, wherein the proportion of molecules of the compound having deuterium at the —R₆ position is greater than 70% of molecules in the composition.

In some embodiments of any of the above composition, wherein the proportion of molecules of the compound having deuterium at the —R₆ position is greater than 60% of molecules in the composition.

In some embodiments of any of the above composition, wherein the proportion of molecules of the compound having deuterium at the —R₆ position is greater than 50% of molecules in the composition.

In some embodiments of the above mixture, wherein the compound having deuterium at the —R₆ deuterium-enriched —H site is

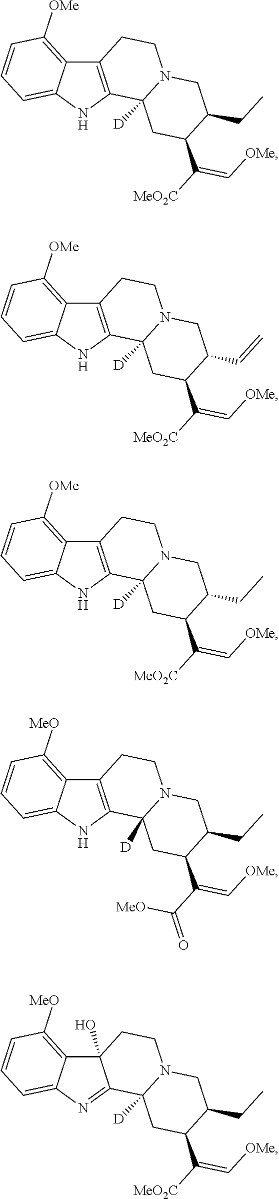 [SMILES: [2H][C@@]12C[C@H](/C(=C\OC)C(=O)OC)[C@H](CC)CN1CCc1c2[nH]c2cccc(OC)c12 ; [2H][C@@]12C[C@H](/C(=C\OC)C(=O)OC)[C@@H](C=C)CN1CCc1c2[nH]c2cccc(OC)c12 ; [2H][C@@]12C[C@H](/C(=C\OC)C(=O)OC)[C@@H](CC)CN1CCc1c2[nH]c2cccc(OC)c12 ; [2H][C@]12C[C@H](/C(=C\OC)C(=O)OC)[C@H](CC)CN1CCc1c2[nH]c2cccc(OC)c12 ; [2H][C@@]12C[C@H](/C(=C\OC)C(=O)OC)[C@H](CC)CN1CC[C@@]1(O)C2=Nc2cccc(OC)c21]

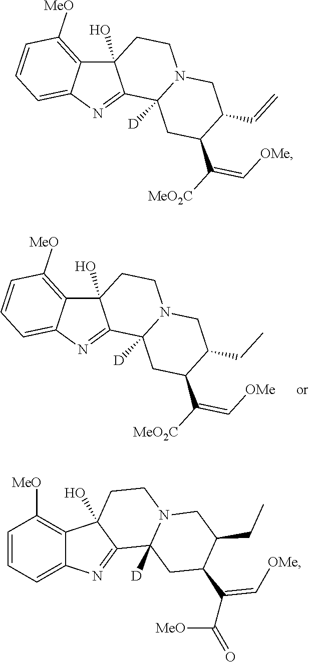 [SMILES: [2H][C@@]12C[C@H](/C(=C\OC)C(=O)OC)[C@@H](C=C)CN1CC[C@@]1(O)C2=Nc2cccc(OC)c21 ; [2H][C@@]12C[C@H](/C(=C\OC)C(=O)OC)[C@@H](CC)CN1CC[C@@]1(O)C2=Nc2cccc(OC)c21 ; [2H][C@]12C[C@H](/C(=C\OC)C(=O)OC)[C@H](CC)CN1CC[C@@]1(O)C2=Nc2cccc(OC)c21]

or a pharmaceutically acceptable salt or ester thereof.

In some embodiments of the above mixture, wherein the compound having deuterium at the —R₆ deuterium-enriched —H site is

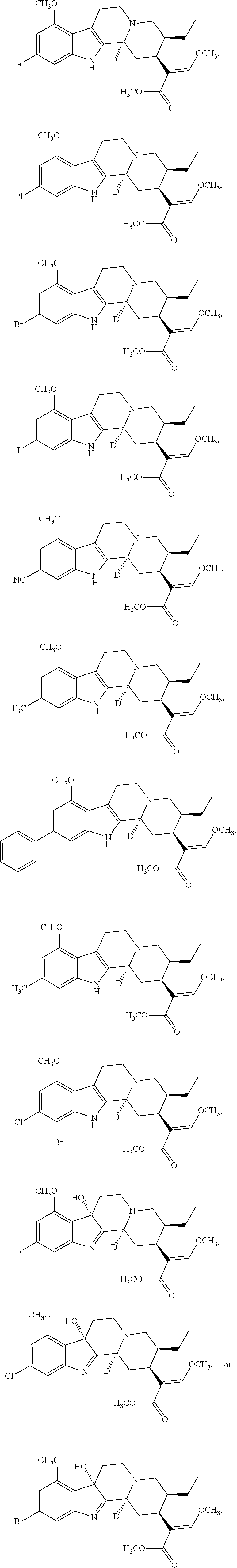

or a pharmaceutically acceptable salt or ester thereof.

In some embodiments of the above mixture, wherein the compound having deuterium at the —R₆ deuterium-enriched —H site is

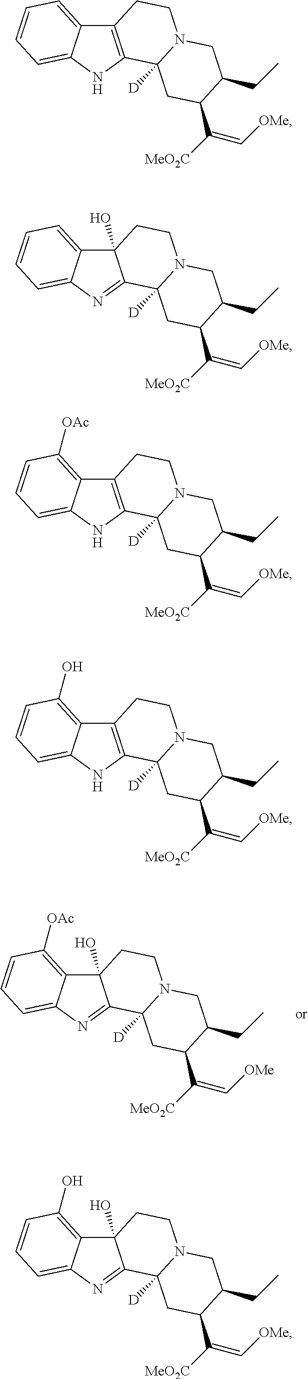 [SMILES: [2H][C@@]12C[C@H](/C(=C\OC)C(=O)OC)[C@H](CC)CN1CCc1c2[nH]c2ccccc12 ; [2H][C@@]12C[C@H](/C(=C\OC)C(=O)OC)[C@H](CC)CN1CC[C@@]1(O)C2=Nc2ccccc21 ; [2H][C@@]12C[C@H](/C(=C\OC)C(=O)OC)[C@H](CC)CN1CCc1c2[nH]c2cccc(OC(C)=O)c12 ; [2H][C@@]12C[C@H](/C(=C\OC)C(=O)OC)[C@H](CC)CN1CCc1c2[nH]c2cccc(O)c12 ; [2H][C@@]12C[C@H](/C(=C\OC)C(=O)OC)[C@H](CC)CN1CC[C@@]1(O)C2=Nc2cccc(OC(C)=O)c21 ; [2H][C@@]12C[C@H](/C(=C\OC)C(=O)OC)[C@H](CC)CN1CC[C@@]1(O)C2=Nc2cccc(O)c21]

or a pharmaceutically acceptable salt or ester thereof.

In some embodiments of any of the above composition, the composition further comprising a carrier.

In some embodiments of any of the above composition, the composition wherein the carrier is a pharmaceutically acceptable carrier.

In some embodiment of any of the above composition, the composition further comprising an NMDA receptor antagonist, an NMDA receptor partial agonist, a neurokinin 1 receptor antagonist, a neurokinin 2 receptor antagonist, a neurokinin 3 receptor antagonist, a DOR agonist, naloxone, methylnaltrexone, a selective serotonin reuptake inhibitor or a serotonin-norepinephrine reuptake inhibitor.

In some embodiments of any of the above composition, the composition wherein the NMDA receptor antagonist is ibogaine or noribogaine.

In some embodiments of any of the above compositions, the compound wherein at least one of H₁—H₁₁ is a deuterium-enriched —H site and R₆ is a deuterium-enriched —H site.

In some embodiments of any of the above compositions, the compound wherein at least one of H₁-H₁₁ is a deuterium-enriched —H site and R₆ is alkyl or aryl.

In some embodiments of any of the above compositions, the compound wherein each of H₁-H₁₂ is —H and R₆ is alkyl or aryl.

In some embodiments of any of the above compositions, the compound wherein each of H₁-H₁₁ is —H and R₆ is a deuterium-enriched —H site.

In some embodiments of any of the above compositions, the compound wherein R₆ is an alkyl, aryl, deuterium or hydrogen.

In some embodiments of any of the above recited compounds, H₁—H₁₁ are each independently —H or a deuterium-enriched —H site.

In some embodiments of any of the above recited compounds, H₁—H₁₁ are each independently —H or -D.

In some embodiments of any of the above recited compounds, R₆ is —H or a deuterium-enriched —H site.

In some embodiments of any of the above recited compounds, R₆ is —H or —D.

In some embodiments of any of the above recited compounds, wherein

R₆ is C₂-C₁₂ alkyl.

In some embodiments of any of the above recited compounds, wherein R₆ is C₃-C₁₂ alkyl.

In some embodiments of any of the above recited compounds, wherein R₆ is C₄-C₁₂ alkyl.

In some embodiments, the above pharmaceutical composition further comprising an NMDA receptor antagonist, an NMDA receptor partial agonist, a neurokinin 1 receptor antagonist, a neurokinin 2 receptor antagonist, a neurokinin 3 receptor antagonist, a DOR agonist, naloxone, methylnaltrexone, a selective serotonin reuptake inhibitor or a serotonin-norepinephrine reuptake inhibitor.

In some embodiments, a method of activating a mu-opioid receptor comprising contacting the mu-opioid receptor with the composition the present invention.

In some embodiments, a method of antagonizing a delta-opioid receptor and/or a kappa-opioid receptor comprising contacting the delta-opioid receptor and/or the kappa-opioid receptor with the composition of the present invention.

In some embodiments, a method of treating a subject afflicted with pain, a depressive disorder, or a mood disorder, or an anxiety disorder comprising administering an effective amount of the composition of the present invention to the subject so as to thereby treat the subject afflicted with pain, a depressive disorder, a mood disorder, or an anxiety disorder.

In some embodiments, a method of treating a subject afflicted with pain comprising administering to the subject an effective amount of an NMDA receptor antagonist, an NMDA receptor partial agonist, a neurokinin 1 receptor antagonist, or a delta-opioid receptor agonist and an effective amount of the composition of the present invention so as to thereby treat the subject afflicted with pain.

In some embodiments, a method of treating a subject afflicted with a depressive disorder or mood disorder comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist, or a delta-opioid receptor agonist and an effective amount of the composition of the present invention so as to thereby treat the subject afflicted with the depressive disorder or mood disorder.

In some embodiments, a method of treating a subject afflicted with an anxiety disorder comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist, or a delta-opioid receptor agonist and an effective amount of the composition of the present invention so as to thereby treat the subject afflicted with the anxiety disorder.

In some embodiments, a method of treating a subject afflicted with borderline personality disorder comprising administering an effective amount of the composition of the present invention to the subject so as to treat the subject afflicted with the borderline personality disorder.

In some embodiments, a method of treating a subject afflicted with a substance use disorder comprising administering an effective amount of the composition of the present invention to the subject so as to treat the subject afflicted with the substance use disorder.

In some embodiments, a method of treating a subject afflicted with opioid use disorder comprising administering an effective amount of the composition of the present invention to the subject so as to treat the subject afflicted with the opioid use disorder.

In some embodiments, a method of treating a subject afflicted with opioid withdrawal symptoms comprising administering an effective amount of the composition of the present invention to the subject so as to treat the subject afflicted with the opioid withdrawal symptoms.

In some embodiments, a method of treating a subject afflicted with borderline personality disorder comprising administering to the subject an effective amount of an NMDA receptor antagonist, an NMDA receptor partial agonist, a neurokinin 1 receptor antagonist, or a DOR agonist and an effective amount of the composition of the present invention so as to thereby treat the subject afflicted with borderline personality disorder.

In some embodiments, a method of treating a subject afflicted with opioid use disorder or opioid withdrawal symptoms comprising administering to the subject an effective amount of an NMDA receptor antagonist, an NMDA receptor partial agonist, or a neurokinin 1 receptor antagonist and an effective amount of the composition of the present invention so as to thereby treat the subject afflicted with the opioid use disorder or opioid withdrawal symptoms.

In some embodiments, a method of treating a subject afflicted with opioid use disorder or opioid withdrawal symptoms comprising administering to the subject an effective amount of naloxone or methylnaltrexone and an effective amount of the composition of the present invention so as to thereby treat the subject afflicted with the opioid use disorder or opioid withdrawal symptoms.

In some embodiments, a method of treating a subject afflicted with pain, a depressive disorder, a mood disorder, an anxiety disorder, or borderline personality disorder, comprising administering to the subject an effective amount of naloxone or methylnaltrexone and an effective amount of the composition of the present invention so as to thereby treat the subject afflicted with pain, the depressive disorder, the mood disorder, the anxiety disorder, or borderline personality disorder.

In some embodiments, a method of treating a subject afflicted with a depressive disorder, a mood disorder, an anxiety disorder, or borderline personality disorder, comprising administering to the subject an effective amount of a selective serotonin reuptake inhibitor or a serotonin-norepinephrine reuptake inhibitor and an effective amount of the composition of the present invention so as to thereby treat the subject afflicted with the depressive disorder, the mood disorder, the anxiety disorder, or borderline personality disorder.

A process for producing a composition comprising a compound having the structure:

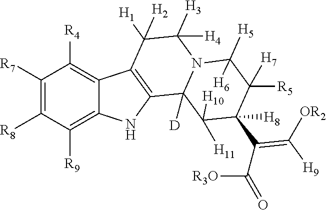

wherein D represents a hydrogen site which is deuterium-enriched, comprising
(i) reacting the compound having the following structure:

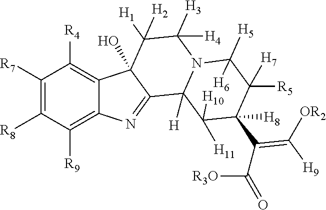

with an acid in a first suitable solvent so as to thereby produce the compound having the following structure:

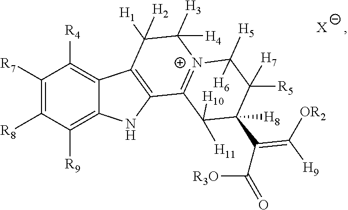

wherein X⁻ is a suitable counter ion; and
(ii) reacting the product of step (i) with NaBD₄ in a second suitable solvent under conditions sufficient to thereby produce the compound.

The above process may be applied to prepare any of the R₆ deuterium enriched compounds disclosed herein.

The present invention further provides a process for producing a composition comprising a compound having the structure:

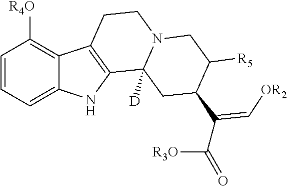

wherein
R₂ is -alkyl;
R₃ is -alkyl;
R₄ is -alkyl; and
R₅ is alkyl or alkenyl,
   wherein D represents a hydrogen site which is deuterium-enriched, comprising
   (i) reacting the compound having the following structure:

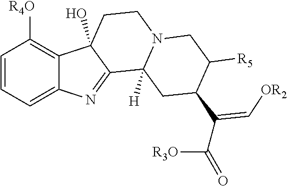

with an acid in a first suitable solvent so as to thereby produce the compound having the following structure:

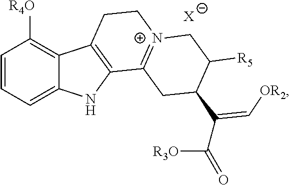

wherein X⁻ is the counter ion corresponding to the acid used; and (ii) reacting the product of step (i) with NaBD₄ in a second suitable solvent under conditions sufficient to thereby produce the composition comprising the compound.

In some embodiments, the process further comprising

(i) reacting the compound having the following structure:

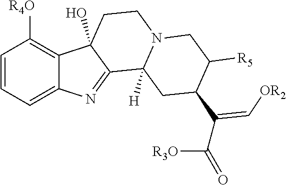

with HCl, HBr, HI, acetic acid, trifluoroacetic acid, sulfuric acid, phosphoric acid, formic acid, perchioric acid or nitric acid in a first suitable solvent so as to thereby produce the compound having the following structure:

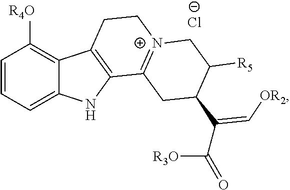

and
(ii) reacting the product of step (i) with NaBD₄ in a second suitable solvent under conditions sufficient to thereby produce the composition comprising the compound.

In some embodiments of the above process wherein the composition produced comprises a compound having the structure:

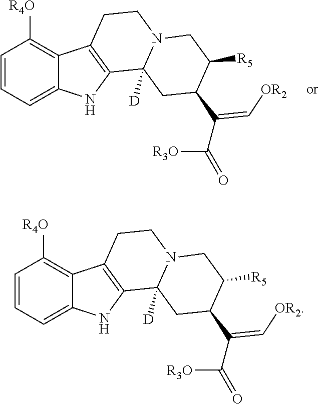

In some embodiments of the above process wherein the second suitable solvent is a deuterated solvent.

In some embodiments of the above process wherein the second suitable solvent is methanol-d₄.

In some embodiments of the above process wherein the second suitable solvent is methanol-OD.

In some embodiments of the above process wherein the composition produced comprises a compound having the structure:

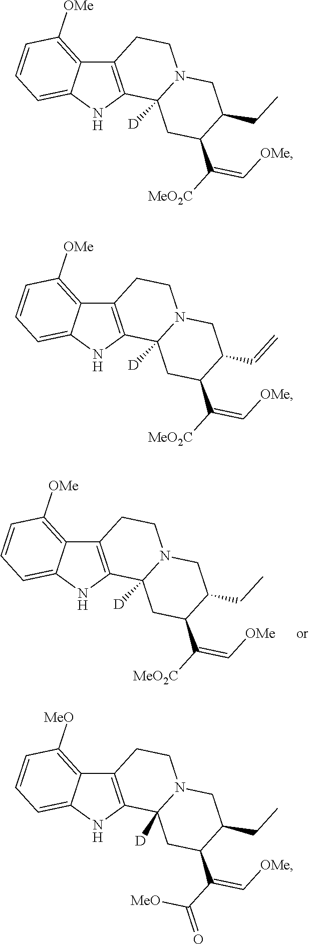

wherein D represents a hydrogen which is deuterium-enriched.

In some embodiments of the above process wherein the composition produced comprises a compound having the structure:

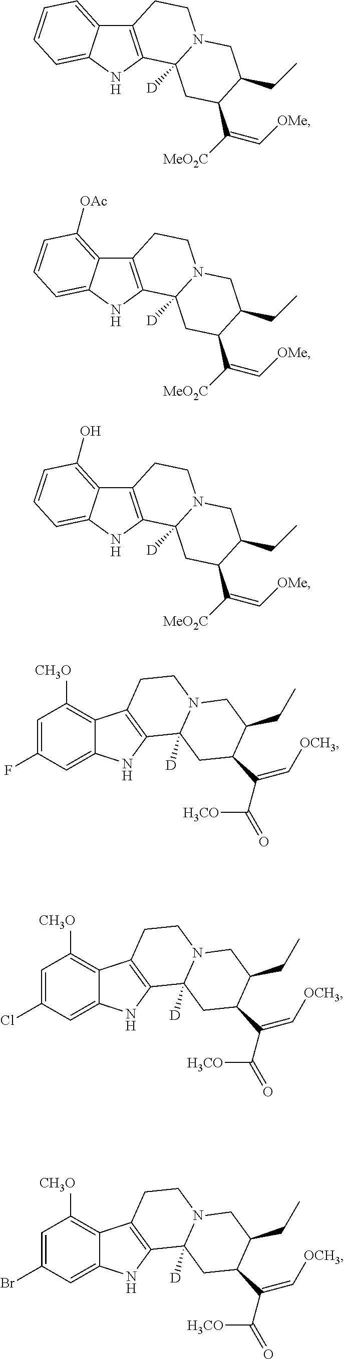

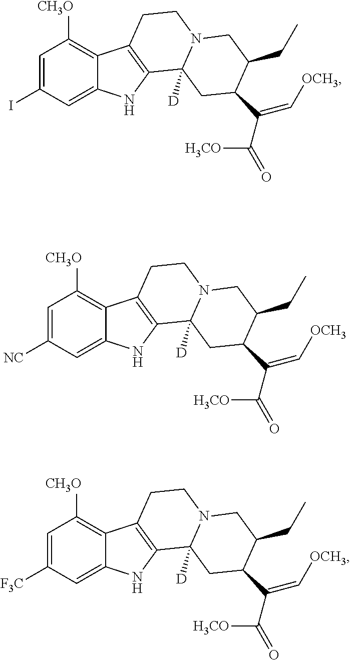

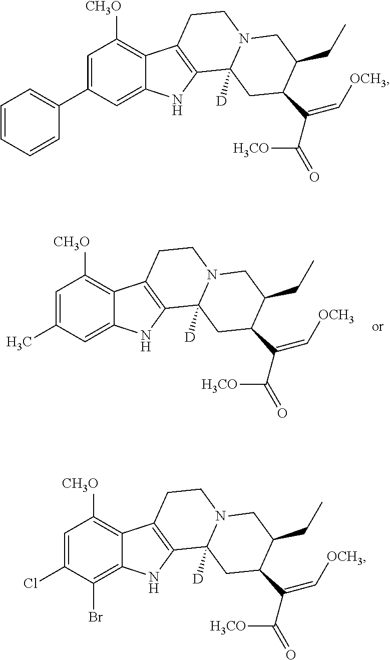

wherein D represents a hydrogen which is deuterium-enriched.

A process for producing a composition comprising a compound having the structure:

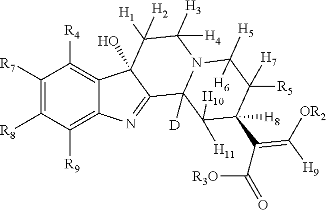

wherein D represents a deuterium-enriched site, comprising
(i) reacting the compound having the following structure:

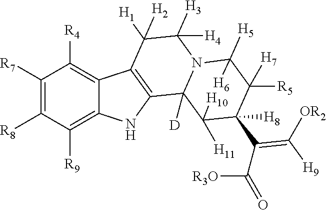

with an oxidizing agent in a suitable solvent under conditions sufficient to thereby produce the compound.

The above process may be applied to prepare any of the R₆ deuterium enriched 7-hydroxy compounds disclosed herein.

The present invention further provides a process for producing a composition comprising a compound having the structure:

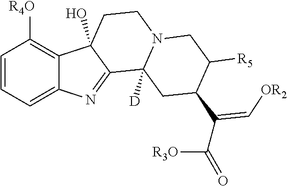

wherein
R₂ is -alkyl;
R₃ is -alkyl;
R₄ is -alkyl; and
R₅ is alkyl or alkenyl,
   wherein D represents a deuterium-enriched site, comprising
   (i) reacting the compound having the following structure:

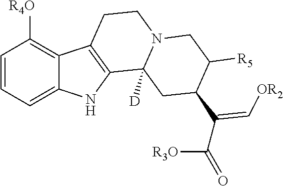

with an oxidizing agent in a suitable solvent under conditions sufficient to thereby produce the composition comprising the compound.

In some embodiments of the above process wherein the oxidizing agent is potassium peroxymonosulfate.

In some embodiments of the above process wherein the reaction occurs in the presence of a base.

In some embodiments of the above process wherein the base is sodium bicarbonate.

In some embodiments of the above process wherein the suitable solvent is acetone.

In some embodiments of the above process wherein the composition produced comprises a compound having the structure:

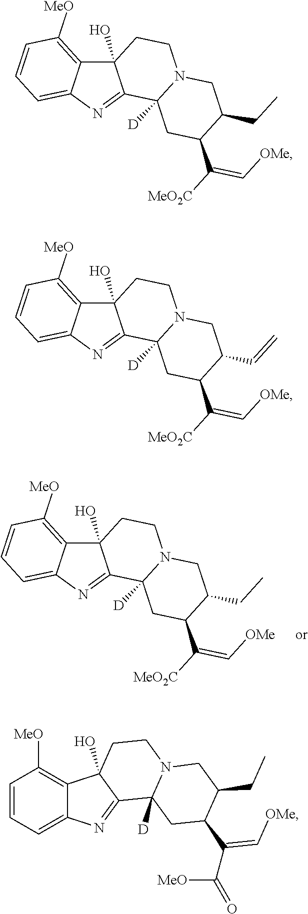

wherein D represents a hydrogen which is deuterium-enriched.

In some embodiments of the above process wherein the composition produced comprises a compound having the structure:

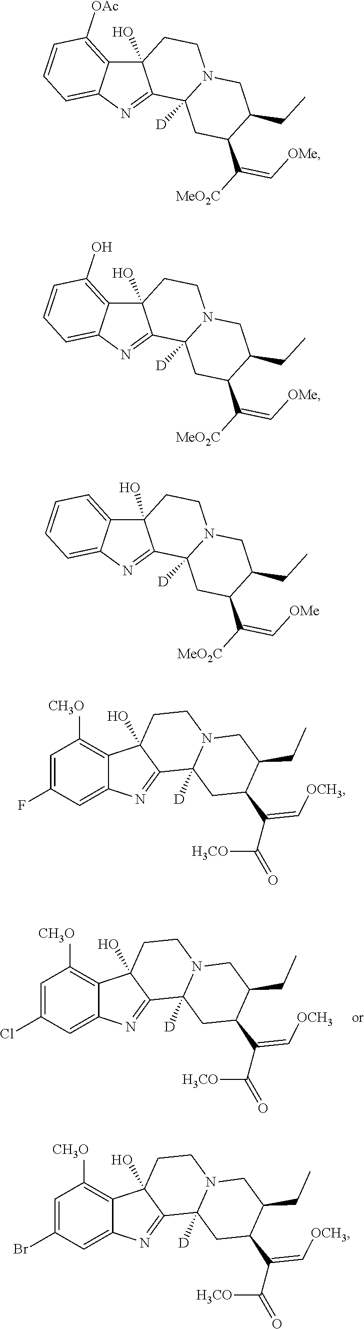

wherein D represents a hydrogen which is deuterium-enriched.

The present invention also provides a method for systemic in vivo delivery of a first composition which comprises a first carrier and a first compound having the structure:

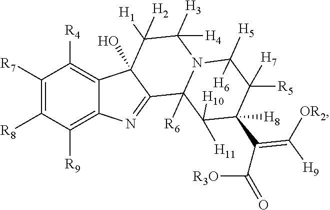

to a subject, the method comprising administering to the subject a second composition which comprises a second carrier and a second compound having the structure:

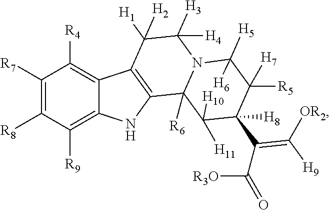

so as to thereby deliver the first compound to the subject.

The above method may be applied to deliver any of the 7-hydroxy compounds disclosed herein.

The present invention also provides a method for systemic in vivo delivery of a first composition which comprises a first carrier and a first compound having the structure:

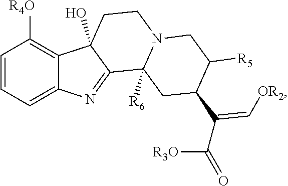

wherein
R₂ is —H or -alkyl;
R₃ is —H or -alkyl;
R₄ is —H or -alkyl;
R₅ is alkyl or alkenyl; and
R₆ is alkyl, aryl, or a deuterium-enriched —H site;
   to a subject, the method comprising administering to the subject a second composition which comprises a second carrier and a second compound having the structure:

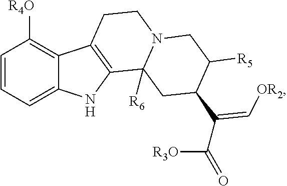

wherein
R₂ is —H or -alkyl;
R₃ is —H or -alkyl;
R₄ is —H or -alkyl;
R₅ is alkyl or alkenyl; and
R₆ is alkyl, aryl, or a deuterium-enriched —H site
   so as to thereby deliver the first compound to the subject.

In some embodiments of the above method, wherein the systemic in vivo delivery of the first composition which comprises the compound occurs without substantially delivering a third compound having the structure:

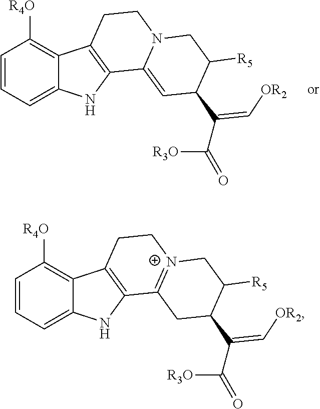

wherein
R₂ is —H or -alkyl;
R₃ is —H or -alkyl;
R₄ is —H or -alkyl; and
R₅ is alkyl or alkenyl.

In some embodiments of the above method, wherein the subject is afflicted with pain, a depressive disorder, a mood disorder, an anxiety disorder, or substance use disorder.

In some embodiments of the above method, wherein administration of the second composition is effective to treat the subject afflicted with the pain, depressive disorder, mood disorder, anxiety disorder, anxiety disorder, or substance use disorder.

In some embodiments of the above method, wherein the second composition is orally administered to the subject.

In some embodiments of the above method, wherein 10-30 mg of the second composition is administered to the subject.

In some embodiments of the above method, wherein 30-100 mg of the second composition compound is administered to the subject.

In some embodiments of the above method, wherein 100-300 mg of the second composition is administered to the subject.

In some embodiments of the above method, wherein the second compound has the structure:

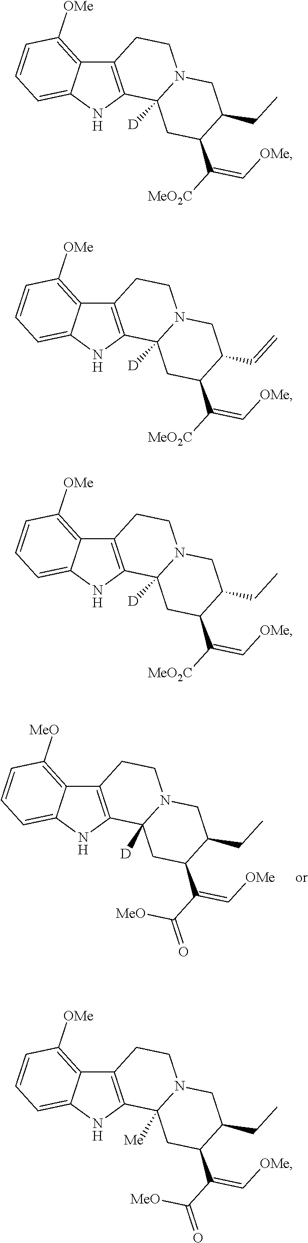

wherein D represents a deuterium-enriched —H site or a pharmaceutically acceptable salt or ester thereof.

In some embodiments of the above method, wherein the second compound has the structure:

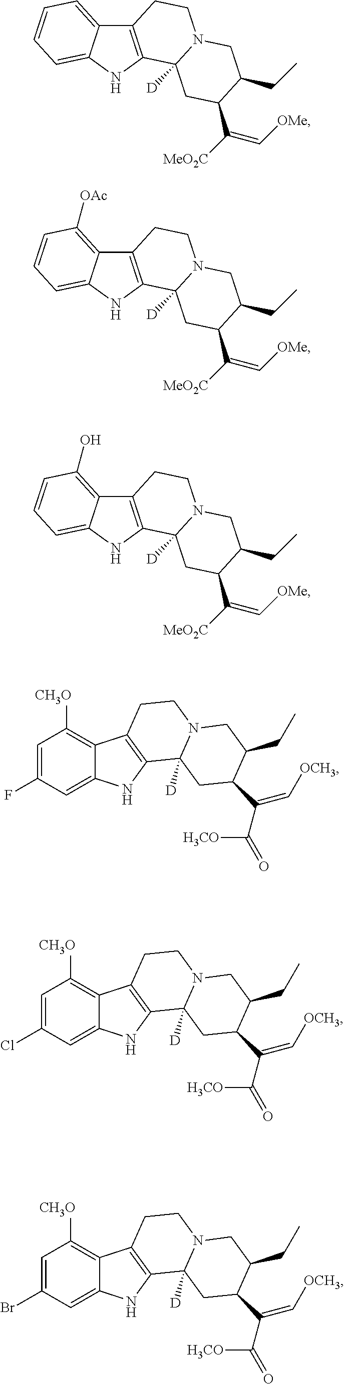

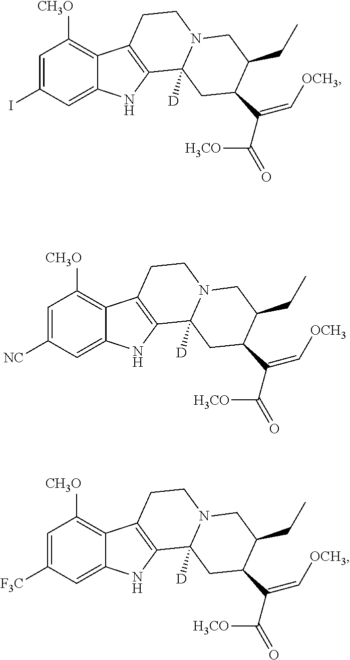

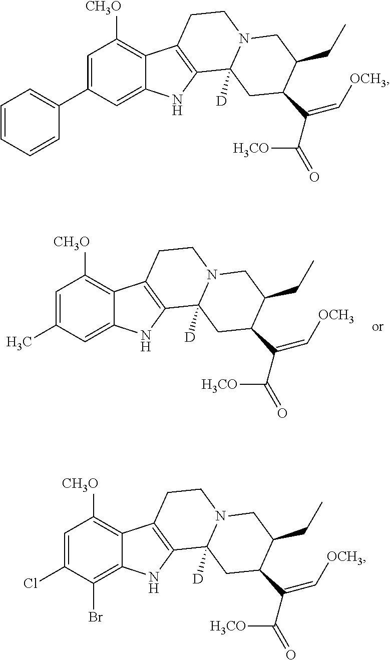

wherein D represents a deuterium-enriched —H site or a pharmaceutically acceptable salt or ester thereof.

In some embodiments of the above method, wherein the first compound has the structure:

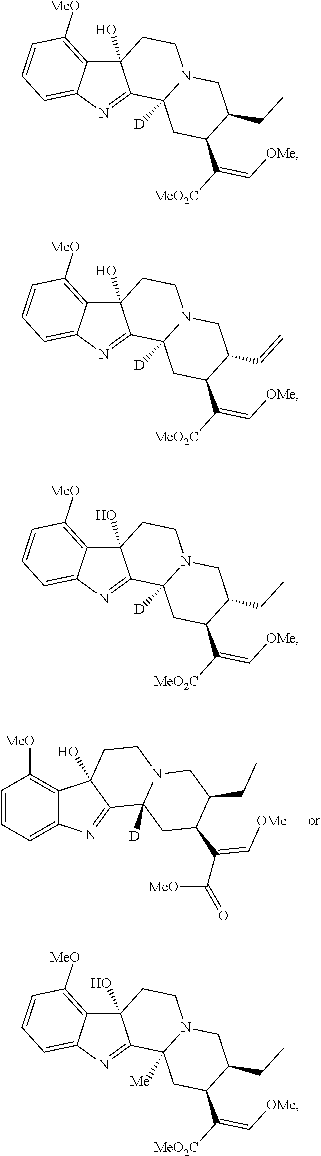 [SMILES: [2H][C@@]12C[C@H](/C(=C\OC)C(=O)OC)[C@H](CC)CN1CC[C@@]1(O)C2=Nc2cccc(OC)c21 ; [2H][C@@]12C[C@H](/C(=C\OC)C(=O)OC)[C@@H](C=C)CN1CC[C@@]1(O)C2=Nc2cccc(OC)c21 ; [2H][C@@]12C[C@H](/C(=C\OC)C(=O)OC)[C@@H](CC)CN1CC[C@@]1(O)C2=Nc2cccc(OC)c21 ; [2H][C@]12C[C@H](/C(=C\OC)C(=O)OC)[C@H](CC)CN1CC[C@@]1(O)C2=Nc2cccc(OC)c21 ; CC[C@@H]1CN2CC[C@@]3(O)C(=Nc4cccc(OC)c43)[C@]2(C)C[C@@H]1/C(=C\OC)C(=O)OC]

or a pharmaceutically acceptable salt or ester thereof.

In some embodiments of the above method, wherein the first has the structure:

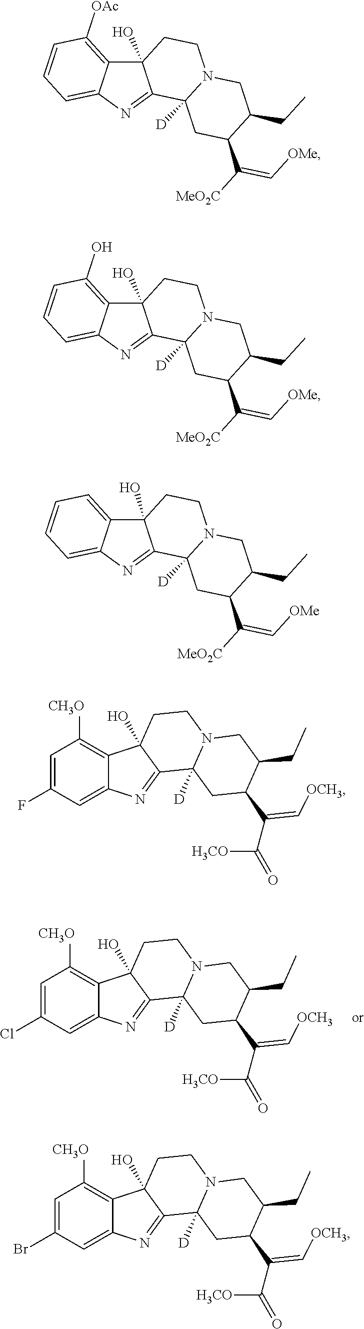

or a pharmaceutically acceptable salt or ester thereof.

In some embodiments of the above method, wherein formation of the toxic metabolite 3-dehydromitragynine is attenuated within the subject.

In another embodiment, R₁ is O—(C₁₋₅ alkyl). In one embodiment, R₁ is O—(C₁₋₁₀ alkyl). In one embodiment, R₁ is O—(C₁ alkyl).

In another embodiment, R₁ is —O(CO)—(C₁₋₅ alkyl). In one embodiment, R₁ is —O(CO)—(C₁₋₁₀ alkyl).

In one embodiment, R₂ is (C₁₋₅ alkyl). In one embodiment, R₂ is (C₁₋₁₀ alkyl). In one embodiment, R₂ is (C₁ alkyl).

In one embodiment, R₃ is (C₁₋₅ alkyl). In one embodiment, R₃ is (C₁₋₁₀ alkyl). In one embodiment, R₃ is (C₁ alkyl).

In one embodiment, R₄ is (C₁₋₅ alkyl). In one embodiment, R₄ is (C₁₋₁₀ alkyl). In one embodiment, R₄ is (C₁ alkyl).

In some embodiments, wherein when the composition contains more than the naturally occurring number of molecules of the compound having deuterium at one or more sites then the composition is a deuterium-enriched composition.

In some embodiments, wherein when R₆ is —H, the composition is enriched in the compound having deuterium at the R₆ position.

In some embodiments, wherein the pharmaceutical composition is enriched in the compound that contains deuterium in place of —H.

In some embodiments, the method wherein the subject is afflicted with pain, a, depressive disorder, a mood disorder, or an anxiety disorder.

In some embodiments, the anxiety disorder includes, but is not limited to, anxiety, generalized anxiety disorder (GAD), panic disorder, social phobia, social anxiety disorder, acute stress disorder, obsessive-compulsive disorder (OCD), or post-traumatic stress disorder (PTSD).

In some embodiments, the depressive disorder includes, but is not limited to, depression, major depression, dysthymia, cyclothymia, postpartum depression, seasonal affective disorder, atypical depression, psychotic depression, bipolar disorder, premenstrual dysphoric disorder, situational depression or adjustment disorder with depressed mood. Depressive disorders can also include other mood disorders and is not limited to the above list.

In some embodiments, the NMDA receptor antagonist is an arylcyclohexylamine, dextromorphinan or adamantane.

In some embodiments, the NMDA receptor antagonist is dextromethorphan, dextrorphan, dextrallorphan, memantine, amantadine, rimantadine, nitromemantine (YQW-36), ketamine (and its analogs, e.g. tiletamine), phencyclidine (and its analogs, e.g. tenocyclidine, eticyclidine, rolicyclidine), methoxetamine (and its analogs), gacyclidine (GK-11), neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CGP 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A) or AP-7.

In some embodiments, the NMDA receptor partial agonist is NRX-1074 or rapastinel (GLYX-13).

In some embodiments, the neurokinin 1 receptor antagonist is aprepitant, fosaprepitant, casopitant, maropitant, vestipitant, vofopitant, lanepitant, orvepitant, ezlopitant, netupitant, rolapitant, L-733060, L-703606, L-759274, L-822429, 1-760735, L-741671, L-742694, 1-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888 or GR 82334.

In some embodiments, the neurokinin 2 receptor antagonist is saredutant, ibodutant, nepadutant, GR-159897 or MEN-10376.

In some embodiments, the neurokinin 3 receptor antagonist is osanetant, talnetant, SB-222200 or SB-218795.

In some embodiments, the DOR agonist is tianeptine, (+)BW373U86, SNC-80, SNC-121, SNC-162, DPI-287, DPI-3290, DPI-221, TAN-67, KN-127, AZD2327, JNJ-20788560, NIH11082, RWJ-394674, ADL5747, ADL5859, UFP-512, AR-M100390, SB-235863 or 7-spiroindanyloxymorphone.

Potassium peroxymonosulfate is used as an oxidizing agent and is commercially available from DuPont under the trade name OXONE® as a component of a triple salt with the formula KHSO₅.0.5KHSO₄.0.5K₂SO₄. In some embodiments, the potassium peroxymonosulfate source is OXONE®.

In some embodiments, OXONE® refers to solution of KHSO₅.0.5KHSO₄. 0.5 K₂SO₄ in water. The concentration of OXONE® may be, but is not limited to, about 10%, 20%, 30%, 40% or 50%.

The term “MOR agonist” is intended to mean any compound or substance that activates the mu-opioid receptor (MOR). The agonist may be a partial, full or super agonist.

The term “DOR agonist” is intended to mean any compound or substance that activates the delta-opioid receptor (DOR). The agonist may be a partial, full or super agonist.

The term “KOR agonist” is intended to mean any compound or substance that activates the kappa-opioid receptor (KOR). The agonist may be a partial, full or super agonist.

The term “super agonist” is intended to mean a compound or substance that activates a receptor with a greater maximal response (higher E) than said receptor's primary endogenous ligand.

The term “MOR antagonist” is intended to mean any compound or substance that blocks or dampens activity of the mu-opioid receptor (MOR). In some instances, the MOR antagonist disrupts the interaction and inhibits the function of an agonist or inverse agonist at the MOR. The antagonist may be a competitive, non-competitive, uncompetitive, or silent antagonist.

The term “DOR antagonist” is intended to mean any compound or substance that blocks or dampens activity of the delta-opioid receptor (DOR). In some instances, the DOR antagonist disrupts the interaction and inhibits the function of an agonist or inverse agonist at the DOR. The antagonist may be a competitive, non-competitive, uncompetitive, or silent antagonist.

The term “KOR antagonist” is intended to mean any compound or substance that blocks or dampens activity of the kappa-opioid receptor (KOR). In some instances, the KOR antagonist disrupts the interaction and inhibits the function of an agonist or inverse agonist at the KOR. The antagonist may be a competitive, non-competitive, uncompetitive, or silent antagonist.

The present invention also provides a compound having the structure:

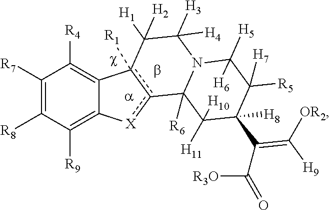

or a salt or ester thereof, for use in treating a subject afflicted with pain, a depressive disorder, an anxiety disorder or a mood disorder.

The present invention also provides a compound having the structure:

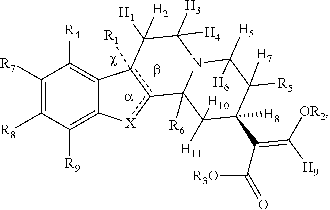

or a salt or ester thereof, for use in treating a subject afflicted with opioid withdrawal symptoms, opioid use disorder, and other substance use disorders, for example, substance use disorders associated with use of alcohol, cocaine, amphetamines, and/or other substances of abuse.

The present invention further provides a pharmaceutical composition comprising an amount of a compound having the structure:

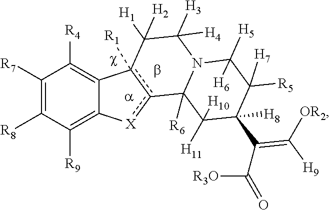

or a salt or ester thereof, for use in treating a subject afflicted with pain, a depressive disorder, an anxiety disorder or a mood disorder.

The present invention also provides a compound having the structure:

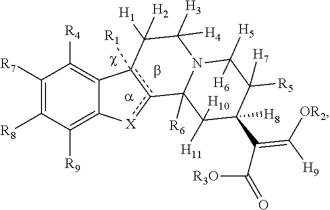

or a salt or ester thereof, for use as an add-on therapy or in combination with an NMDA receptor antagonist, an NMDA receptor partial agonist, a neurokinin 1 receptor antagonist, a neurokinin 2 receptor antagonist, a neurokinin 3 receptor antagonist, or a DOR agonist in treating a subject afflicted with pain, a depressive disorder, an anxiety disorder or a mood disorder.

In some embodiments, a package comprising:
a) a first pharmaceutical composition comprising an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist and a pharmaceutically acceptable carrier;
b) a second pharmaceutical composition comprising an amount of any compound of the present invention, or a salt or ester thereof; and
c) instructions for use of the first and second pharmaceutical compositions together to treat a subject afflicted with pain, a depressive disorder, an anxiety disorder or a mood disorder.

In some embodiments, a therapeutic package for dispensing to, or for use in dispensing to, a subject afflicted with pain, a depressive disorder, an anxiety disorder or a mood disorder, which comprises:

a) one or more unit doses, each such unit dose comprising:
(i) an amount of any compound of the present invention, or a salt or ester thereof; and
(ii) an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist,
wherein the respective amounts of said compound and said agonist or antagonist in said unit dose are effective, upon concomitant administration to said subject, to treat the subject, and
   (b) a finished pharmaceutical container therefor, said container containing said unit dose or unit doses, said container further containing or comprising labeling directing the use of said package in the treatment of said subject.

The therapeutic package of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

A pharmaceutical composition in unit dosage form, useful in treating a subject afflicted with pain, a depressive disorder, an anxiety disorder, or a mood disorder, which comprises:
(i) an amount of any compound of the present invention, or a salt or ester thereof; and
(ii) an amount of an NMDA receptor antagonist, an NMDA receptor partial agonist, a neurokinin 1 receptor antagonist, a neurokinin 2 receptor antagonist, a neurokinin 3 receptor antagonist, or a DOR agonist,
wherein the respective amounts of said compound and said agonist or antagonist in said composition are effective, upon concomitant administration to said subject of one or more of said unit dosage forms of said composition, to treat the subject.

The pharmaceutical composition of the above embodiment, wherein the respective amounts of said compound and said agonist or antagonist in said unit dose when taken together is more effective to treat the subject than when compared to the administration of said compound in the absence of said agonist or antagonist or the administration of said agonist or antagonist in the absence of said compound.

In some embodiments of the present method, compound, package, use or pharmaceutical composition, the compound has the structure:

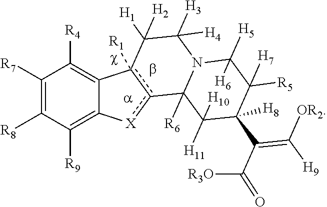

In some embodiments, a pharmaceutically acceptable salt of any of the above compounds of the present invention.

In some embodiments, a salt of the compound of the present invention is used in any of the above methods, uses, packages or compositions.

In some embodiments, a pharmaceutically acceptable salt of the compound of the present invention is used in any of the above methods, uses, packages or compositions.

In some embodiments, an ester of the compound of the present invention is used in any of the above methods, uses, packages or compositions.

Any of the above compounds may be used in any of the disclosed methods, uses, packages or pharmaceutical compositions.

Any of the compounds used in the disclosed methods, uses, packages or pharmaceutical compositions may be replaced with any other compound disclosed in the present invention.

Any of the above generic compounds may be used in any of the disclosed methods, uses, packages or compositions.

Techniques and method for making the compounds of the present application may be found in 1) International Publication No. WO 2017/165738 A1; 2) International Publication No. WO 2016/176657 A1; or 3) the WO International Publication of PCT International Application No. PCT/US2019/046677, the contents of each of which are hereby incorporated by reference. A person skilled in the art may use the techniques disclosed therein to prepare compounds which are not enriched in deuterium and thereafter use the techniques disclosed herein to prepare deuterium analogs thereof.

Except where otherwise specified, the structure of a compound of this invention includes an asymmetric carbon atom, it is understood that the compound occurs as a racemate, racemic mixture, scalemic mixtures and isolated single enantiomers. All such isomeric forms of these compounds are expressly included in this invention. Except where otherwise specified, each stereogenic carbon may be of the R or S configuration. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wiley, Pub. John Wiley & Sons, N Y, 1981. For example, the resolution may be carried out by preparative chromatography on a chiral column.

Except where otherwise specified, the subject invention is intended to include all isotopes of atoms occurring on the compounds disclosed herein. Isotopes include those atoms having the same atomic number but different mass numbers. By way of general example and without limitation, isotopes of hydrogen include tritium and deuterium. Isotopes of carbon include C-13 and C-14.

It will be noted that any notations of a carbon in, structures throughout this application, when used without further notation, are intended to represent all isotopes of carbon, such as ¹²C, ¹³C, or ¹⁴C. Furthermore, any compounds containing ¹³C or ¹⁴C may specifically have the structure of any of the compounds disclosed herein.

It will also be noted that any notations of a hydrogen (H) in structures throughout this application, when used without further notation, are intended to represent all isotopes of hydrogen, such as ¹H, ²H (D), or ³H (T) except where otherwise specified. Furthermore, any compounds containing ²H or ³H may specifically have the structure of any of the compounds disclosed herein except where otherwise specified.

Isotopically-labeled compounds can generally be prepared by conventional techniques known to those skilled in the art using appropriate isotopically-labeled reagents in place of the non-labeled reagents employed.

Deuterium (²H or D) is a stable, non-radioactive isotope of hydrogen and has an atomic weight of 2.0144. Hydrogen atom in a compound naturally occurs as a mixture of the isotopes ¹H (hydrogen or protium), D (²H or deuterium), and T (³H or tritium). The natural abundance of deuterium is 0.0156%. Thus, in a composition comprising molecules of a naturally occurring compound, the level of deuterium at a particular hydrogen atom site in that compound is expected to be 0.0156%. Thus, a composition comprising a compound with a level of deuterium at any site of hydrogen atom in the compound that has been enriched to be greater than its natural abundance of 0.0156% is novel over its naturally occurring counterpart.

As used herein, a hydrogen at a specific site in a compound is “deuterium-enriched” if the amount of deuterium at the specific site in the compound is more than the abundance of deuterium naturally occurring at that specific site in view of all of the molecules of the compound in a defined universe such as a composition or sample. Naturally occurring as used above refers to the abundance of deuterium which would be present at a relevant site in a compound if the compound was prepared without any affirmative step to enrich the abundance of deuterium. Thus, at a “deuterium-enriched” site in a compound, the abundance of deuterium at that site can range from more than 0.0156% to 100%. Examples of ways to obtain a deuterium-enriched site in a compound are exchanging hydrogen with deuterium or synthesizing the compound with deuterium-enriched starting materials.

In the compounds used in the method of the present invention, the substituents may be substituted or unsubstituted, unless specifically defined otherwise.

In the compounds used in the method of the present invention, alkyl, heteroalkyl, monocycle, bicycle, aryl, heteroaryl and heterocycle groups can be further substituted by replacing one or more hydrogen atoms with alternative non-hydrogen groups. These include, but are not limited to, halo, hydroxy, mercapto, amino, carboxy, cyano and carbamoyl.

It is understood that substituents and substitution patterns on the compounds used in the method of the present invention can be selected by one of ordinary skill in the art to provide compounds that are chemically stable and that can be readily synthesized by techniques known in the art from readily available starting materials. If a substituent is itself substituted with more than one group, it is understood that these multiple groups may be on the same carbon or on different carbons, so long as a stable structure results.

In choosing the compounds used in the method of the present invention, one of ordinary skill in the art will recognize that the various substituents, i.e. R₁, R₂, etc. are to be chosen in conformity with well-known principles of chemical structure connectivity.

As used herein, “alkyl” is intended to include both branched and straight-chain saturated aliphatic hydrocarbon groups having the specified number of carbon atoms. Thus, C₁-Cₙ as in “C₁-Cₙ alkyl” is defined to include groups having 1, 2, . . . , n−1 or n carbons in a linear or branched arrangement, and specifically includes methyl, ethyl, propyl, butyl, pentyl, hexyl, heptyl, isopropyl, isobutyl, sec-butyl and so on. An embodiment can be C₁-C₁₂ alkyl, C₂-C₁₂ alkyl, C₃-C₁₂ alkyl, C₄-C₁₁ alkyl and so on. An embodiment can be C₁-C₈ alkyl, C₂-C₈ alkyl, C₃-C₈ alkyl, C₄-C₈ alkyl and so on. “Alkoxy” represents an alkyl group as described above attached through an oxygen bridge.

The term “alkenyl” refers to a non-aromatic hydrocarbon radical, straight or branched, containing at least 1 carbon to carbon double bond, and up to the maximum possible number of non-aromatic carbon-carbon double bonds may be present. Thus, C₂-Cₙ alkenyl is defined to include groups having 1, 2 . . . , n−1 or n carbons. For example, “C₂-C₆ alkenyl” means an alkenyl radical having 2, 3, 4, 5, or 6 carbon atoms, and at least 1 carbon-carbon double bond, and up to, for example, 3 carbon-carbon double bonds in the case of a C₆ alkenyl, respectively. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated. An embodiment can be C₂-C₁₂ alkenyl or C₂-C₈ alkenyl.

The term “alkynyl” refers to a hydrocarbon radical straight or branched, containing at least 1 carbon to carbon triple bond, and up to the maximum possible number of non-aromatic carbon-carbon triple bonds may be present. Thus, C₂-Cₙ alkynyl is defined to include groups having 1, 2 . . . , n−1 or n carbons. For example, “C₂-C₆ alkynyl” means an alkynyl radical having 2 or 3 carbon atoms, and 1 carbon-carbon triple bond, or having 4 or 5 carbon atoms, and up to 2 carbon-carbon triple bonds, or having 6 carbon atoms, and up to 3 carbon-carbon triple bonds. Alkynyl groups include ethynyl, propynyl and butynyl. As described above with respect to alkyl, the straight or branched portion of the alkynyl group may contain triple bonds and may be substituted if a substituted alkynyl group is indicated. An embodiment can be a C₂-Cₙ alkynyl. An embodiment can be C₂-C₁₂ alkynyl or C₃-C₅ alkynyl.

As used herein, “hydroxyalkyl” includes alkyl groups as described above wherein one or more bonds to hydrogen contained therein are replaced by a bond to an —OH group. In some embodiments, C₁-C₁₂ hydroxyalkyl or C₁-C₆ hydroxyalkyl. C₁-Cₙ as in “C₁-Cₙ alkyl” is defined to include groups having 1, 2, . . . , n−1 or n carbons in a linear or branched arrangement (e.g. C₁-C₂ hydroxyalkyl, C₁-C₃ hydroxyalkyl, C₁-C₄ hydroxyalkyl, C₁-C₅ hydroxyalkyl, or C₁-C₆ hydroxyalkyl). For example, C₁-C₆, as in “C₁-C₆ hydroxyalkyl” is defined to include groups having 1, 2, 3, 4, 5, or 6 carbons in a linear or branched alkyl arrangement wherein a hydrogen contained therein is replaced by a bond to an —OH group.

As used herein, “heteroalkyl” includes both branched and straight-chain saturated aliphatic hydrocarbon groups having the specified number of carbon atoms and at least 1 heteroatom within the chain or branch.

As used herein, “monocycle” includes any stable polyatomic carbon ring of up to 10 atoms and may be unsubstituted or substituted. Examples of such non-aromatic monocycle elements include but are not limited to: cyclobutyl, cyclopentyl, cyclohexyl, and cycloheptyl. Examples of such aromatic monocycle elements include but are not limited to: phenyl.

As used herein, “bicycle” includes any stable polyatomic carbon ring of up to 10 atoms that is fused to a polyatomic carbon ring of up to atoms with each ring being independently unsubstituted or substituted. Examples of such non-aromatic bicycle elements include but are not limited to: decahydronaphthalene. Examples of such aromatic bicycle elements include but are not limited to: naphthalene.

As used herein, “aryl” is intended to mean any stable monocyclic, bicyclic or polycyclic carbon ring of up to 10 atoms in each ring, wherein at least one ring is aromatic, and may be unsubstituted or substituted. Examples of such aryl elements include but are not limited to: phenyl, p-toluenyl (4-methylphenyl), naphthyl, tetrahydro-naphthyl, indanyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or polycyclic ring of up to 10 atoms in each ring, wherein at least one ring is aromatic and contains from 1 to 4 heteroatoms selected from the group consisting of O, N and S. Bicyclic aromatic heteroaryl groups include phenyl, pyridine, pyrimidine or pyridazine rings that are (a) fused to a 6-membered aromatic (unsaturated) heterocyclic ring having one nitrogen atom; (b) fused to a 5- or 6-membered aromatic (unsaturated) heterocyclic ring having two nitrogen atoms; (c) fused to a 5-membered aromatic (unsaturated) heterocyclic ring having one nitrogen atom together with either one oxygen or one sulfur atom; or (d) fused to a 5-membered aromatic (unsaturated) heterocyclic ring having one heteroatom selected from O, N or S.

Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazoiyl, isoxazolyl, naphthpyridinyl, oxadiazolyl, oxazolyl, oxazoline, isoxazoline, oxetanyl, pyranyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrazolyl, tetrazolopyridyl, thiadiazolyl, thiazolyl, thienyl, triazolyl, azetidinyl, aziridinyl, 1,4-dioxanyl, hexahydroazepinyl, dihydrobenzoimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, tetrahydrothienyl, acridinyl, carbazolyl, cinnolinyl, quinoxalinyl, pyrrazolyl, indolyl, benzotriazolyl, benzothiazolyl, benzoxazolyl, isoxazolyl, isothiazolyl, furanyl, thienyl, benzothienyl, benzofuranyl, quinolinyl, isoquinolinyl, oxazolyl, isoxazolyl, indolyl, pyrazinyl, pyridazinyl, pyridinyl, pyrimidinyl, pyrrolyl, tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle”, “heterocyclyl” or “heterocyclic” refers to a mono- or poly-cyclic ring system which can be saturated or contains one or more degrees of unsaturation and contains one or more heteroatoms. Preferred heteroatoms include N, O, and/or S, including N-oxides, sulfur oxides, and dioxides. Preferably the ring is three to ten-membered and is either saturated or has one or more degrees of unsaturation. The heterocycle may be unsubstituted or substituted, with multiple degrees of substitution being allowed. Such rings may be optionally fused to one or more of another “heterocyclic” ring(s), heteroaryl ring(s), aryl ring(s), or cycloalkyl ring(s). Examples of heterocycles include, but are not limited to, tetrahydrofuran, pyran, 1,4-dioxane, 1,3-dioxane, piperidine, piperazine, pyrrolidine, morpholine, thiomorpholine, tetrahydrothiopyran, tetrahydrothiophene, 1,3-oxathiolane, and the like.

The term “ester” is intended to a mean an organic compound containing the R—O—CO—R′ group.

The term “substitution”, “substituted” and “substituent” refers to a functional group as described above in which one or more bonds to a hydrogen atom contained therein are replaced by a bond to non-hydrogen or non-carbon atoms, provided that normal valencies are maintained and that the substitution results in a stable compound. Substituted groups also include groups in which one or more bonds to a carbon(s) or hydrogen(s) atom are replaced by one or more bonds, including double or triple bonds, to a heteroatom. Examples of substituent groups include the functional groups described above, and halogens (i.e., F, Cl, Br, and I); alkyl groups, such as methyl, ethyl, n-propyl, isopropryl, n-butyl, tert-butyl, and trifluoromethyl; hydroxyl; alkoxy groups, such as methoxy, ethoxy, n-propoxy, and isopropoxy; aryloxy groups, such as phenoxy; arylalkyloxy, such as benzyloxy (phenylmethoxy) and p-trifluoromethylbenzyloxy (4-trifluoromethylphenylmethoxy); heteroaryloxy groups; sulfonyl groups, such as trifluoromethanesulfonyl, methanesulfonyl, and p-toluenesulfonyl; nitro, nitrosyl; mercapto; sulfanyl groups, such as methylsulfanyl, ethylsulfanyl and propylsulfanyl; cyano; amino groups, such as amino, methylamino, dimethylamino, ethylamino, and diethylamino; and carboxyl. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

The compounds used in the method of the present invention may be prepared by techniques well known in organic synthesis and familiar to a practitioner ordinarily skilled in the art. However, these may not be the only means by which to synthesize or obtain the desired compounds.

The compounds used in the method of the present invention may be prepared by techniques described in Vogel's Textbook of Practical Organic Chemistry, A. T. Vogel, A. R. Tatchell, B. S. Furnis, A. J. Hannaford, P. W. G. Smith, (Prentice Hall) 5^{th} Edition (1996), March's Advanced Organic Chemistry: Reactions, Mechanisms, and Structure, Michael B. Smith, Jerry March, (Wiley-Interscience) 5^{th} Edition (2007), and references therein, which are incorporated by reference herein. However, these may not be the only means by which to synthesize or obtain the desired compounds.

The various R groups attached to the aromatic rings of the compounds disclosed herein may be added to the rings by standard procedures, for example those set forth in Advanced Organic Chemistry: Part B: Reactions and Synthesis, Francis Carey and Richard Sundberg, (Springer) 5th ed. Edition. (2007), the content of which is hereby incorporated by reference.

Another aspect of the invention comprises a compound used in the method of the present invention as a pharmaceutical composition.

As used herein, the term “pharmaceutically active agent” means any substance or compound suitable for administration to a subject and furnishes biological activity or other direct effect in the treatment, cure, mitigation, diagnosis, or prevention of disease, or affects the structure or any function of the subject. Pharmaceutically active agents include, but are not limited to, substances and compounds described in the Physicians' Desk Reference (PDR Network, LLC; 64th edition; Nov. 15, 2009) and “Approved Drug Products with Therapeutic Equivalence Evaluations” (U.S. Department of Health and Human Services, 30^{th} edition, 2010), which are hereby incorporated by reference. Pharmaceutically active agents which have pendant carboxylic acid groups may be modified in accordance with the present invention using standard esterification reactions and methods readily available and known to those having ordinary skill in the art of chemical synthesis. Where a pharmaceutically active agent does not possess a carboxylic acid group, the ordinarily skilled artisan will be able to design and incorporate a carboxylic acid group into the pharmaceutically active agent where esterification may subsequently be carried out so long as the modification does not interfere with the pharmaceutically active agent's biological activity or effect.

The compounds used in the method of the present invention may be in a salt form. As used herein, a “salt” is a salt of the instant compounds which has been modified by making acid or base salts of the compounds. In the case of compounds used to treat a disease or medical disorder, the salt is pharmaceutically acceptable. Examples of pharmaceutically acceptable salts include, but are not limited to, mineral or organic acid salts of basic residues such as amines; alkali or organic salts of acidic residues such as phenols; alkali or organic salts of acidic residues such as carboxylic acids. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the sodium, potassium, or lithium salts, and the like. Carboxylate salts are the sodium, potassium, or lithium salts, and the like. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

As used herein, “treating” means preventing, slowing, halting, or reversing the progression of a disease. Treating may also mean improving one or more symptoms of a disease.

The compounds used in the method of the present invention may be administered in various forms, including those detailed herein. The treatment with the compound may be a component of a combination therapy or an adjunct therapy, i.e. the subject or patient in need of the drug is treated or given another drug for the disease in conjunction with one or more of the instant compounds. This combination therapy can be sequential therapy where the patient is treated first with one drug and then the other or the two drugs are given simultaneously. These can be administered independently by the same route or by two or more different routes of administration depending on the dosage forms employed.

As used herein, a “pharmaceutically acceptable carrier” is a pharmaceutically acceptable solvent, suspending agent or vehicle, for delivering the instant compounds to the animal or human. The carrier may be liquid or solid and is selected with the planned manner of administration in mind. Liposomes are also a pharmaceutically acceptable carrier, as are capsules, coatings and various syringes.

The dosage of the compounds administered in treatment will vary depending upon factors such as the pharmacodynamic characteristics of a specific chemotherapeutic agent and its mode and route of administration; the age, sex, metabolic rate, absorptive efficiency, health and weight of the recipient; the nature and extent of the symptoms; the kind of concurrent treatment being administered; the frequency of treatment with; and the desired therapeutic effect.

A dosage unit of the compounds used in the method of the present invention may comprise a single compound or mixtures thereof with additional agents. The compounds can be administered in oral dosage forms as tablets, capsules, pills, powders, granules, elixirs, tinctures, suspensions, syrups, and emulsions. The compounds may also be administered in intravenous (bolus or infusion), intraperitoneal, subcutaneous, or intramuscular form, or introduced directly, e.g. by injection, topical application, or other methods, into or onto a site of disease, all using dosage forms well known to those of ordinary skill in the pharmaceutical arts.

The compounds used in the method of the present invention can be administered in admixture with suitable pharmaceutical diluents, extenders, excipients, or carriers (collectively referred to herein as a pharmaceutically acceptable carrier) suitably selected with respect to the intended form of administration and as consistent with conventional pharmaceutical practices. The unit will be in a form suitable for oral, rectal, topical, intravenous or direct injection or parenteral administration. The compounds can be administered alone or mixed with a pharmaceutically acceptable carrier. This carrier can be a solid or liquid, and the type of carrier is generally chosen based on the type of administration being used. The active agent can be co-administered in the form of a tablet or capsule, liposome, as an agglomerated powder or in a liquid form. Examples of suitable solid carriers include lactose, sucrose, gelatin and agar. Capsule or tablets can be easily formulated and can be made easy to swallow or chew; other solid forms include granules, and bulk powders. Tablets may contain suitable binders, lubricants, diluents, disintegrating agents, coloring agents, flavoring agents, flow-inducing agents, and melting agents. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

Techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al. 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol. 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol (Gilbert S. Banker, Christopher T. Rhodes, Eds.). All of the aforementioned publications are incorporated by reference herein.

Tablets may contain suitable binders, lubricants, disintegrating agents, coloring agents, flavoring agents, flow-inducing agents, and melting agents. For instance, for oral administration in the dosage unit form of a tablet or capsule, the active drug component can be combined with an oral, non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol and the like. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds used in the method of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines. The compounds may be administered as components of tissue-targeted emulsions.

The compounds used in the method of the present invention may also be coupled to soluble polymers as targetable drug carriers or as a prodrug. Such polymers include polyvinylpyrrolidone, pyran copolymer, polyhydroxylpropylmethacrylamide-phenol, polyhydroxyethylasparta-midephenol, or polyethyleneoxide-polylysine substituted with palmitoyl residues. Furthermore, the compounds may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Gelatin capsules may contain the active ingredient compounds and powdered carriers, such as lactose, starch, cellulose derivatives, magnesium stearate, stearic acid, and the like. Similar diluents can be used to make compressed tablets. Both tablets and capsules can be manufactured as immediate release products or as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

For oral administration in liquid dosage form, the oral drug components are combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water-soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol. Suitable pharmaceutical carriers are described in Remington's Pharmaceutical Sciences, 17th ed., 1989, a standard reference text in this field.

The compounds used in the method of the present invention may also be administered in intranasal form via use of suitable intranasal vehicles, or via transdermal routes, using those forms of transdermal skin patches well known to those of ordinary skill in that art. To be administered in the form of a transdermal delivery system, the dosage administration will generally be continuous rather than intermittent throughout the dosage regimen.

Parenteral and intravenous forms may also include minerals and other materials to make them compatible with the type of injection or delivery system chosen.

Each embodiment disclosed herein is contemplated as being applicable to each of the other disclosed embodiments. Thus, all combinations of the various elements described herein are within the scope of the invention. Any of the disclosed generic or specific compounds may be applicable to any of the disclosed compositions, processes or methods.

This invention will be better understood by reference to the Experimental Details which follow, but those skilled in the art will readily appreciate that the specific experiments detailed are only illustrative of the invention as described more fully in the claims, which follow thereafter.

### Experimental Details

General Considerations. Reagents and solvents were obtained from commercial sources and were used without further purification unless otherwise stated. Reactions were monitored by TLC using solvent mixtures appropriate to each reaction. All column chromatography was performed on silica gel (40-63 μm). Preparative TLC was conducted on glass plates coated with a 1 mm silica layer. Nuclear magnetic resonance spectra were recorded on Bruker 400 or 500 MHz instruments, as indicated. Chemical shifts are reported as δ values in ppm referenced to CDCl₃ (¹H NMR=7.26 and ¹³C NMR=77.16) or methanol-d₄ (¹H NMR=3.31 and ¹³C NMR=49.00). Multiplicity is indicated as follows: s (singlet); d (doublet); t (triplet); dd (doublet of doublets); td (triplet of doublets); dt (doublet of triplets); ddd (doublet of doublet of doublets); m (multiplet); br (broad). All carbon peaks are rounded to one decimal place unless such rounding would cause two close peaks to become identical; in these cases, two decimal places are retained. Low-resolution mass spectra were recorded on an Advion quadrupole instrument (ionization mode: APCI+). Percent deuteration was determined by mass spectrometry on a high-resolution quadrupole-time-of-flight instrument (ionization mode: ESI+) by quantitative comparison of the isotope pattern of deuterated compounds to controls having natural isotopic abundance.

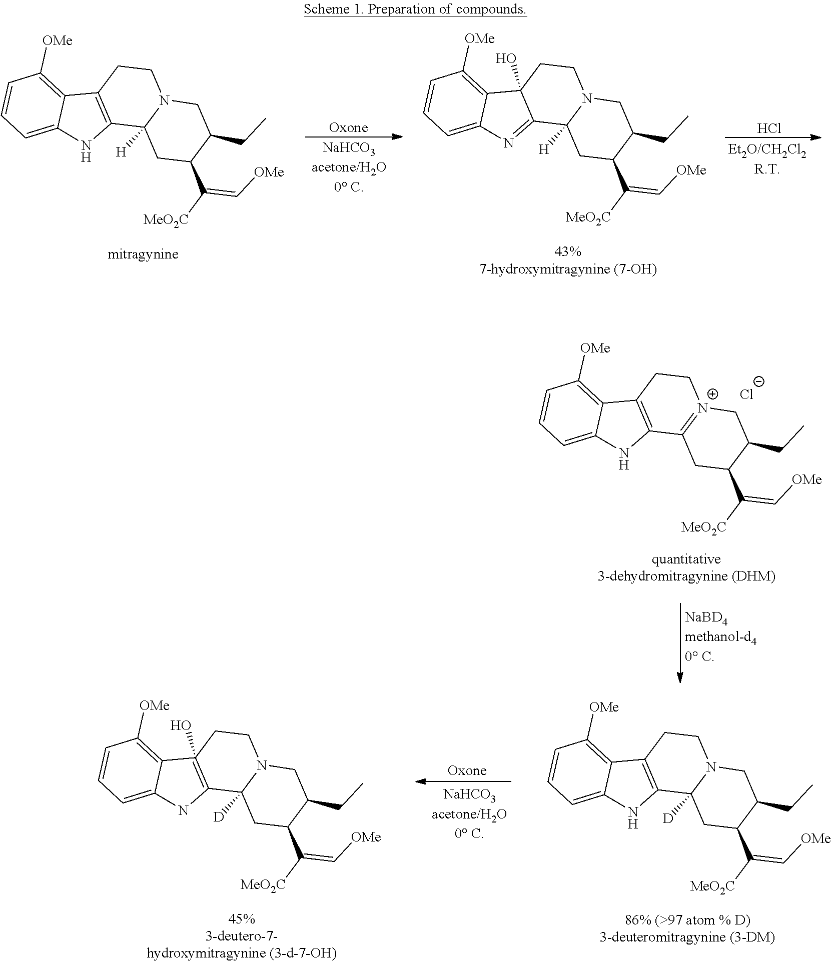

Mitragynine. Mitragynine free base was obtained by extraction from powdered Mitragyna speciosa leaves as previously described (Kruegel et al. 2016). Spectral and physical properties were in agreement with those previously reported (Kruegel et al. 2016).

7-Hydroxymitragynine (7-OH) Procedure 1). Mitragynine (1.99 g, 5.00 mmol) was dissolved in acetone (100 ML), saturated aqueous NaHCO₃ (10 mL) was added, and the mixture was cooled to 0° C. A solution of Oxone monopersulfate (2KHSO₅.KHSO₄.K₂SO₄; 2.31 g, 3.75 mmol) in water (10 mL) was then added dropwise over 35 minutes and the mixture left to stir at 0° C. After 45 minutes, additional Oxone monopersulfate (769 mg, 1.25 mmol) in water (3.3 mL) was added over ˜2 minutes and stirring was continued at 0° C. for an additional 15 minutes. At this time, the reaction was diluted with water (150 mL) and extracted with EtOAc (3×50 mL). The combined organics were washed with brine (50 mL), dried over Na₂SO₄, and concentrated in vacuo to give the crude product as a tan foam (1.42 g). This material was purified by column chromatography (6:4 hexanes:EtOAc+2% Et₃N) to provide pure 7-hydroxymitragynine as an amorphous, pale-yellow solid (882 mg, 43%). Spectral and physical properties were in agreement with those previously reported (Kruegel et al. 2016).

7-Hydroxymitragynine (7-OH) (Procedure 2—Larger Scale). Mitragynine (9.96 g, 25.00 mmol) was dissolved in acetone (750 mL), saturated aqueous NaHCO₃ (500 mL) was added, and the mixture was cooled to 0° C. A solution of Oxone monopersulfate (2KHSO₅.KHSO₄.K₂SO₄; 15.39 g, 25.00 mmol) in water (250 mL) was then pre-cooled to 0° C. and added dropwise over 30 minutes (mixture was hard to stir at first but became less viscous over the course of the addition). TLC at the end of the Oxone addition showed no starting material so the reaction was worked up (at ˜15 minutes after the end of the addition). EtOAc (500 mL) and water (500 mL) were added to the reaction mixture while it was still stirring at 0° C. and the resulting mixture was then poured into a separatory funnel containing additional water (1,000 mL). The organic later was separated and the aqueous phase extracted with additional EtOAc (2×500 mL). The combined organics were washed with brine (300 mL), dried over Na₂SO₄, and concentrated in vacuo to give the crude product as a yellow ochre foam (7.35 g). This material was purified by silica column chromatography (320 g silica; 600 mL column volume; 60 mL fractions; step gradient: 20%→30%→35%→40%→45%→50%→55% EtOAc in hexanes+2% Et₃N, 1 column volume per step) to provide the following fractions: fractions 49-51=very pale-yellow amorphous solid, 7-hydroxymitragynine+˜2% 7-hydroxycorynantheidine, 1.09 g (11%); fractions 52-64=pale-yellow amorphous solid, 7-hydroxymitragynine, 2.99 g (29%). Spectral properties were in agreement with those previously reported (Kruegel et al. 2016).

3-Dehydromitragynine hydrochloride (DHM) (Procedure 1). To a solution of 7-hydroxymitragynine (746 mg, 1.80 mmol) in anhydrous CH₂Cl₂ (27 mL) under argon was added 2.0 M HCl in Et₂O (9.0 mL) and the resulting mixture was stirred at room temperature for 45 minutes (all solids dissolved to give a transparent yellow solution after 2-3 minutes). The reaction mixture was then concentrated directly in vacuo to give pure 3-dehydromitragynine hydrochloride as a yellow solid (797 mg, quantitative). ¹H NMR (500 MHz, CDCl₃) δ 13.56 (br s, 1H), 7.49 (s, 1H), 7.42 (d, J=8.4 Hz, 1H), 7.25 (t, J=8.0 Hz 1H), 6.38 (d, J=7.7 Hz, 1H), 4.03-3.81 (m, 3H), 3.89 (s, 3H), 3.80-3.65 (m, 1H), 3.76 (s, 3H), 3.65-3.53 (m, 2H), 3.61 (s, 3H), 3.53-3.36 (m, 2H), 3.29 (t, J=12.6 Hz, 1H), 2.10 (br s, 1H), 1.55-1.43 (m, 1H), 1.22-1.10 (m, 1H), 0.98 (t, J=7.4 Hz, 3H).

3-Dehydromitragynine hydrochloride (DHM) (Procedure 2—Larger Scale). To a solution of 7-hydroxymitragynine (14.09 g, 34.00 mmol) in anhydrous CH₂Cl₂ (510 mL) under argon was added 2.0 M HCl in Et₂O (170 mL) (yellow suspension forms and slight warming occurs on HCl addition) and the resulting mixture was stirred at room temperature for 40 minutes (all solids dissolved to give a transparent yellow-orange solution after 2-3 minutes). The reaction mixture was then concentrated directly in vacuo to give pure 3-dehydromitragynine hydrochloride as a yellow solid (15.87 g, quantitative). The NMR spectra of this material were identical to those of material obtained via Procedure 1 above.

3-Deuteromitragynine (3-DM) (Procedure 1). To a solution of 3-dehydromitragynine hydrochloride (606 mg, 1.40 mmol) in methanol-d₄ (28 mL) at 0° C. was added NaBD₄ (293 mg, 7.00 mmol) and the yellow solution was stirred at 0° C. for 20 minutes. The reaction was then diluted with water (100 mL) and extracted with CH₂C₂ (3×50 mL). The combined organics were washed with water (2×50 mL), dried over Na₂SO₄, and concentrated in vacuo to give the crude product as a very pale-yellow foam (0.52 g). This material was purified by column chromatography (8:2 hexanes:EtOAc+2% Et₃N, 4 column volumes→7:3 hexanes:EtOAc+2% Et₃N, 3 column volumes) to provide pure 3-deuteromitragynine as an amorphous, off-white solid (480 mg, 86%). ¹H NMR (500 MHz, CDCl₃) δ 7.70 (br s, 1H), 7.43 (s, 1H), 6.99 (t, J=7.9 Hz, 1H), 6.90 (d, J=7.7 Hz, 1H), 6.46 (d, J=7.7 Hz, 1H), 3.88 (s, 3H), 3.73 (s, 3H), 3.71 (s, 3H), 3.12 (ddd, J=15.8, 11.6, 5.9 Hz, 1H), 3.07-2.89 (m, 4H), 2.58-2.42 (m, 3H), 1.85-1.73 (m, 2H), 1.66-1.58 (m, 1H), 1.25-1.15 (m, 1H), 0.87 (t, J=7.4 Hz, 3H); ¹³C NMR (126 MHz, CDCl₃) δ 169.4, 160.7, 154.6, 137.4, 133.8, 121.9, 117.7, 111.6, 107.9, 104.4, 99.8, 61.6, 60.9 (t, J_{CD}=19.5 Hz), 57.9, 55.4, 53.9, 51.5, 40.8, 40.1, 29.9, 24.1, 19.2, 13.0; HR-MS calcd. for C₂₃H₃₀DN₂O₄ [M+H]⁺ 400.2341, found 400.2332; Deuterium Enrichment=97.5-97.7 atom % D (by HR-MS).

3-Deuteromitragynine (3-DM) (Procedure 2). To a solution of 3-dehydromitragynine hydrochloride (54.1 mg, 0.125 mmol) in MeOH (2.5 mL) at 0° C. was added NaBD₄ (26.2 mg, 0.625 mmol) and the yellow solution was allowed to warm to room temperature and stirred for 25 minutes. The reaction was then diluted with water (10 mL) and extracted with CH₂Cl₂ (3×5 mL). The combined organics were washed with water (2×5 mL), dried over Na₂SO₄, and concentrated in vacuo to give the crude product as a foamy yellow glass (47.2 mg). This material was purified by column chromatography (7:3 hexanes:EtOAc+2% Et₃N) to provide pure 3-deuteromitragynine as an amorphous, yellow solid (39.4 mg, 79%). The NMR spectra of this material were identical to those of material obtained via Procedure 1 above, with the exception of visible residual peaks for undeuterated mitragynine in both the proton and carbon spectra. Deuterium Enrichment=93.5-93.8 atom D (by HR-MS).

3-Deuteromitragynine (3-DM) (Procedure 3). To a solution of 3-dehydromitragynine hydrochloride (14.72 g, 34.00 mmol=15.85 g of crude containing CH₂Cl₂ from last step) in methanol-OD (CH₂OD; 170 mL) at 0° C. was added NaBD₄ (2.85 mg, 68.00 mmol) and the yellow solution (clouds immediately after NaBD₄ addition) was stirred at 0° C. for 20 minutes (effervescence stops after 10 minutes). The reaction was then diluted with water (500 mL) and extracted with CH₂Cl₂ (3×250 mL). The combined organics were washed with water (2×250 mL), dried over Na₂SO₄, and concentrated in vacuo to give the crude product as a pale-yellow foam (14.28 g). This material was purified by silica column chromatography (320 g silica; 600 mL column volume; 60 mL fractions; step gradient: 10% (2 column volumes)→20% (2 column volumes) 30% (4 column volumes) EtOAc in hexanes+2% Et₃N, first 2 column volumes discarded) to provide the following fractions: fractions 19-45=cream-colored amorphous solid, 3-deuteromitragynine, 11.86 g (87%); fractions 17-18+46-55=pale-yellow amorphous solid, impure 3-deuteromitragynine, 0.66 g (˜5%). The NMR spectra of this material were identical to those of material obtained via Procedures 1 and 2 above. Deuterium Enrichment=98.2-98.4 atom % D (by HR-MS).

3-Deutero-7-hydroxymitragynine (3-d-7-OH). 3-Deuteromitragynine (10.0 mg, 0.025 mmol) was dissolved in acetone (0.75 mL), saturated aqueous NaHCO₃ (0.50 mL) was added, and the mixture was cooled to 0° C. A solution of Oxone monopersulfate (2KHSO₅.KHSO₄.K₂SO₄; 15.4 mg, 0.025 mmol) in water (0.25 mL) was then added dropwise over 25 minutes and the mixture was stirred for 20 minutes at 0° C. At this time, the reaction was diluted with water (10 mL) and extracted with EtOAc (3×5 mL). The combined organics were washed with brine (5 mL), dried over Na₂SO₄, and concentrated in vacuo to give the crude product as a pale yellowish-tan, foamy glass (8.3 mg). This material was purified by preparative TLC (1:1 hexanes:EtOAc+Et₃N, 10×20 cm plate) to provide pure 3-deutero-7-hydroxymitragynine as an amorphous tan solid (4.7 mg, 45%). ¹H NMR (500 MHz, CDCl₃) δ 7.44 (s, 1H), 7.29 (t, J=8.0 Hz, 1H), 7.21 (d, J=7.6 Hz, 1H), 6.73 (d, J=8.2 Hz, 1H), 3.87 (s, 3H), 3.80 (s, 3H), 3.69 (s, 3H), 3.08-2.98 (m, 2H), 2.86-2.75 (m, 2H), 2.68-2.59 (m, 2H), 2.48 (dd, J=11.4, 3.0 Hz, 1H), 2.24 (s, 1H), 1.87 (dd, J=13.6, 3.1 Hz, 1H), 1.76-1.63 (m, 2H), 1.63-1.56 (m, 1H), 1.29-1.19 (m, 1H), 0.82 (t, J=7.4 Hz, 3H); ¹³C NMR (126 MHz, CDCl₃) δ 184.3, 169.5, 160.9, 156.0, 155.3, 130.9, 126.6, 114.4, 111.4, 109.0, 81.3, 61.9, 61.2 (t, J_{CD}=19.1 Hz), 58.3, 55.6, 51.5, 50.2, 40.7, 39.4, 36.1, 26.1, 19.1, 13.0; Deuterium Enrichment=>97 atom % D (by NMR).

Additional analogs of mitragynine deuterated at the 3 position can be prepared in an analogous manner, as exemplified by the procedures shown in Scheme 2. Analogs deuterated at the 3 position attenuate metabolic formation of the analogous 3-dehydro oxidized derivatives.

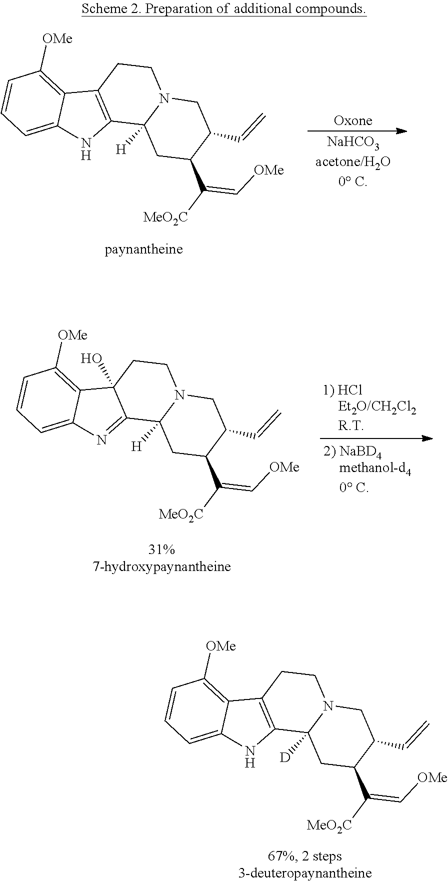 [SMILES: C=C[C@H]1CN2CCc3c([nH]c4cccc(OC)c34)[C@@H]2C[C@@H]1/C(=C\OC)C(=O)OC ; C=C[C@H]1CN2CC[C@@]3(O)C(=Nc4cccc(OC)c43)[C@@H]2C[C@@H]1/C(=C\OC)C(=O)OC ; [2H][C@@]12C[C@H](/C(=C\OC)C(=O)OC)[C@@H](C=C)CN1CCc1c2[nH]c2cccc(OC)c12]

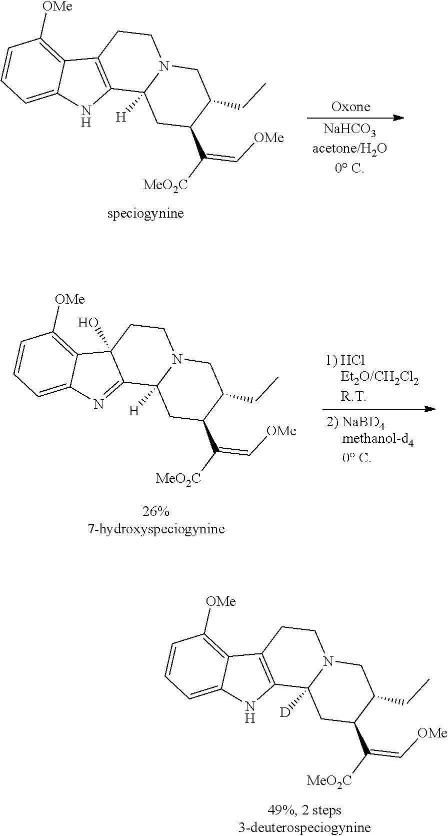 [SMILES: CC[C@H]1CN2CCc3c([nH]c4cccc(OC)c34)[C@@H]2C[C@@H]1/C(=C\OC)C(=O)OC ; CC[C@H]1CN2CC[C@@]3(O)C(=Nc4cccc(OC)c43)[C@@H]2C[C@@H]1/C(=C\OC)C(=O)OC ; [2H][C@@]12C[C@H](/C(=C\OC)C(=O)OC)[C@@H](CC)CN1CCc1c2[nH]c2cccc(OC)c12]

Paynantheine. Paynantheine free base was obtained by extraction from powdered Mitragyna speciosa leaves as previously described (Kruegel et al. 2016). Spectral and physical properties were in agreement with those previously reported (Kruegel et al. 2016).

Speciogynine. Speciogynine free base was obtained by extraction from powdered Mitragyna speciosa leaves as previously described (Kruegel et al. 2016). Spectral, and physical properties were in agreement with those previously reported (Kruegel et al. 2016).

7-Hydroxypaynantheine. A saturated aqueous solution of NaHCO₃ (5 mL) was added to a cooled (0° C.) solution of paynantheine (92 mg, 0.232 mmol) in acetone (7 mL). A precipitate immediately formed and a solution of Oxone monopersulfate (2KHSO₅.KHSO₄.K₂SO₄; 71 mg, 0.115 mmol) in water (2.1 mL) was then added in three portions over a period of 20 minutes. Immediately following the final addition, the reaction was quenched with water (30 mL) and extracted with EtOAc (3×15 mL). The combined organics were washed with brine, dried over Na₂SO₄, and concentrated in vacuo to give the crude product. This material was purified by preparative TLC (1:1 hexanes:EtOAc+5% Et₃N, 20×20 cm plate) to provide 7-hydroxypaynantheine as a pale-yellow solid (30 mg, 31%). ¹H NMR (500 MHz, CDCl₃) δ 7.29 (s, 1H), 7.26 (t, J=8.0 Hz, 1H), 7.15 (d, J=7.6 Hz, 1H), 6.71 (d, J=8.3 Hz, 1H), 5.55 (dt, J=17.7, 9.3 Hz, 1H), 5.01-4.94 (m, 1H), 4.92 (dd, J=10.3, 1.8 Hz, 1H), 3.83 (s, 3H), 3.77 (s, 3H), 3.65 (s, 3H), 3.20 (d, J=10.6 Hz, 1H), 2.99 (d, J=8.2 Hz, 1H), 2.83 (t, J=11.7 Hz, 1H), 2.74-2.66 (m, 1H), 2.63 (d, J=14.3 Hz, 1H), 2.59 (s, 1H), 2.36 (q, J=11.9 Hz, 1H), 2.27 (t, J=11.8 Hz, 1H), 2.01 (d, J=11.5 Hz, 1H), 1.65 (td, J=13.6, 3.8 Hz, 1H), 1.23 (s, 1H), 0.83 (m, 1H).

7-Hydroxyspeciogynine. A saturated aqueous solution of NaHCO₃ (5 mL) was added to a cooled (0° C.) solution of speciogynine (92 mg, 0.231 mmol) in acetone (7 mL). A precipitate immediately formed and a solution of Oxone monopersulfate (2KHSO₅.KHSO₄.K₂SO₄; 71 mg, 0.115 mmol) in water (2.1 mL) was then added in three portions over a period of 20 minutes. Immediately following the final addition, the reaction was quenched with water (30 mL) and extracted with EtOAc (3×15 mL). The combined organics were washed with brine, dried over Na₂SO₄, and concentrated in vacuo to give the crude product. This material was purified by preparative TLC (2:1 hexanes:EtOAc+2% Et₃N, 20×20 cm plate) to provide 7-hydroxyspeciogynine as a pale-orange solid (25 mg, 26% yield). ¹H NMR (500 MHz, CDCl₃) δ 7.34 (d, J=14.2 Hz, 1H), 7.28 (t, J=8.0 Hz, 1H), 7.18 (d, J=7.6 Hz, 1H), 6.73 (d, J=8.2 Hz, 1H), 3.85 (s, 3H), 3.81-3.74 (m, 3H), 3.67 (s, 3H), 3.19 (d, J=9.4 Hz, 1H), 3.14 (dd, J=11.0, 3.6 Hz, 1H), 2.84 (t, J=12.3 Hz, 1H), 2.79-2.70 (m, 1H), 2.66 (d, J=14.2 Hz, 1H), 2.62-2.52 (m, 1H), 2.48-2.14 (m, 2.7H), 2.13-1.77 (m, 2.3H), 1.70 (td, J=13.7, 4.5 Hz, 1H), 1.50-1.33 (m, 1H), 1.07-0.97 (m, 1H), 0.84 (t, J=7.2 Hz, 3H) (Note: partial integrals due to conformers).

3-Deuteropaynantheine. To a solution of 7-hydroxypaynantheine (20 mg, 0.0485 mmol) in anhydrous CH₂Cl₂ (0.75 mL) under argon was added 2M HCl in Et₂O (0.24 mL) at room temperature and the mixture was left to stir. The reaction was monitored by TLC and LR-MS (APCI+) and 20 minutes after disappearance of starting material, the reaction was halted by removal of solvent in vacuo. The resulting crude 3-dehydropaynantheine hydrochloride was used in the next step without purification.

A solution of crude 3-dehydropaynantheine hydrochloride (20 mg, ˜0.0464 mmol) in methanol-d₄ (2 mL) was cooled to 0° C., NaBD₄ (12 mg, 0.287 mmol) was added in one portion, and the resulting solution was left to stir at 0° C. for 25 minutes. The reaction was then poured into water (10 mL) and extracted with CH₂Cl₂ (3×5 mL). The combined organics were washed with water (2×5 mL), dried over Na₂SO₄, and concentrated in vacuo to give the crude product. This material was purified by preparative TLC (3:1 hexanes:EtOAc+2% Et₃N, 20×20 cm plate) to give 3-deuteropaynantheine as a pale-yellow solid (13 mg, 67% for 2 steps). ¹H NMR (400 MHz, CDCl₃) δ 7.72 (s, 1H), 7.33 (s, 1H), 6.99 (t, J=7.9 Hz, 1H), 6.87 (d, J=7.9 Hz, 1H), 6.46 (d, J=7.7 Hz, 1H), 5.58 (ddd, J=18.2, 10.2, 8.3 Hz, 1H), 5.05-4.90 (m, 2H), 3.87 (s, 3H), 3.77 (s, 3H), 3.69 (s, 3H), 3.18 (ddd, J=16.6, 11.2, 5.7 Hz, 1H), 3.11-2.95 (m, 4H), 2.80-2.70 (m, 1H), 2.59 (td, J=11.2, 4.4 Hz, 1H), 2.34-2.24 (m, 1H), 2.07 (d, J=12.3 Hz, 1H), 2.00 (d, J=7.8 Hz, 1H) ppm; LR-MS calcd. for C₂₃H₂₈DN₂O₄ [M+H]⁺ 398.2, found 398.5.

3-Deuterospeciogynine. To a solution of 7-hydroxyspeciogynine (20 mg, 0.0483 mmol) in anhydrous CH₂Cl₂ (0.75 mL) under argon was added 2M HCl in Et₂O (0.24 mL) at room temperature and the mixture was left to stir. The reaction was monitored by TLC and LR-MS (APCI+) and 20 minutes after disappearance of starting material, the reaction was halted by removal of solvent in vacuo. The resulting crude 3-dehydrospeciogynine hydrochloride (19 mg) was used in the next step without purification.

A solution of crude 3-dehydrospeciogynine hydrochloride (12 mg, ˜0.0277 mmol) in methanol-d₄ (1 mL) was cooled to 0° C., NaBD₄ (7.0 mg, 0.167 mmol) was added in one portion, and the resulting solution was left to stir at 0° C. for 25 minutes. The reaction was then poured into water (10 mL) and extracted with CH₂Cl₂ (3×5 mL). The combined organics were washed with water (2×5 mL), dried over Na₂SO₄, and concentrated in vacuo to give the crude product. This material was purified by preparative TLC (3:2 hexanes:EtOAc+2% Et₃N, 20×20 cm plate) to give 3-deuterospeciogynine as a pale-yellow solid (6.0 mg, 49% for 2 steps). ¹H NMR (400 MHz, CDCl₃) δ 7.67 (br s, 1H), 7.36 (br s, 1H), 7.00 (t, J=7.9 Hz, 1H), 6.88 (d, J=8.0 Hz, 1H), 6.46 (d, J=7.7 Hz, 1H), 3.90 (s, 3H), 3.83-3.61 (br m, 6H), 3.28-2.97 (m, 4H), 2.70-2.51 (m, 2H), 2.37-2.23 (m, 1H), 2.15-1.82 (m, 3H), 1.50-1.36 (m, 1H), 1.12-0.97 (m, 1H), 0.86 (t, 3H, J=7.5 Hz) ppm; LR-MS calcd. for C₂₃H₃₀DN₂O₄ [M+H]⁺ 400.2, found 400.7.

### Example 1. Effects of 3-Dehydromitragynine in Rotarod Test in Mice

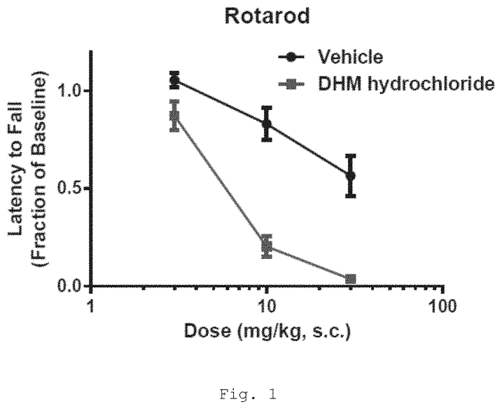

The rotarod test is useful for measuring the motor coordination of rodents and therefore, for identifying test drugs that induce ataxic effects. 3-Dehydromitragynine (DHM) reduces the performance of mice in this test in a dose-dependent manner; indicating an impairment of motor coordination by this compound (FIG. 1).

Animals. This study was conducted using male C57BL/6J mice, 7 weeks of age (n=10/treatment), purchased from The Jackson Laboratory (Bar Harbor, Me.). Animals were housed in groups of five and allowed to acclimate for 1 week prior to testing. Mice had ad libitum access to food and water and were maintained on a 12-hour light/dark cycle. All testing was done in the light cycle.

Drug. DHM hydrochloride was dissolved in double-distilled H₂O containing 10% N-methyl-2-pyrrolidone (NMP). Drug or vehicle was administered subcutaneously 15 minutes prior to the start of behavioral testing at a volume of 10 mL/kg body weight. Dosing was performed cumulatively at 3, 10, and 30 mg/kg, with 1 hour between injections.

Rotarod Testing. On the test day, animals were acclimated to the testing room for 1 hour. An accelerating rotarod (Model 7650, UGO Basile, Comeria, VA, Italy) was used to measure the motor coordination of the animals. Time on the rotarod was measured by stopwatch, starting when animals were placed on the rod and ending at the time the animal fell off the apparatus. Rotarod speed began at 0 rpm and gradually increased to 40 rpm over 5 minutes. Animals received one round of training 24 hours prior to the test date, during which time a baseline was collected. Training consisted of placing the animals back onto the rotarod if an animal fell within 10 seconds of the start of the test or replacement on the rod.

### Example 2. Lethality of 3-Dehydromitragynine in Mice

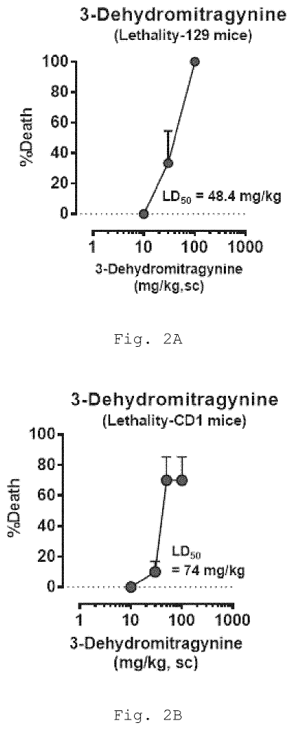

Treatment of mice with 3-dehydromitragynine (DHM) results in death in a dose-dependent manner in two different strains, demonstrating the general toxicity of this metabolite (FIG. 2).

Lethality Assay. Groups of mice (n=6 per dose, 129Sv6 or CD-1 strain) were treated subcutaneously (s.c.) with different doses of DHM and tested for lethality 24 h after drug administration.

### Example 3. Attenuated Formation of 3-Dehydromitragynine in Liver Microsomes

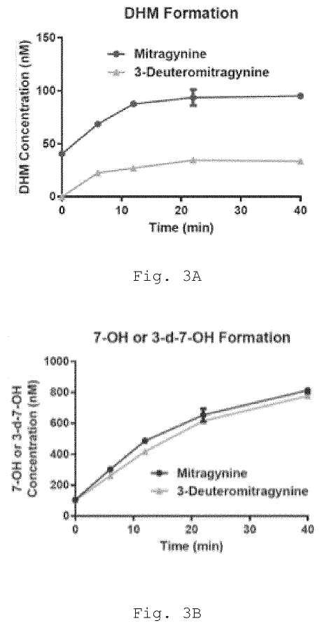

In human liver microsomes (HLM), both 7-hydroxymitragynine (7-OH) and 3-dehydromitragynine (DHM) are formed as metabolites of mitragynine (FIG. 3). Deuteration of the 3 position of mitragynine, as in 3-deuteromitragynine (3-DM), attenuates formation of DHM via a kinetic isotope effect (FIG. 3A), while having no effect on oxidative metabolism at the 7 position to give 3-deutero-7-hydroxymitragynine (3-d-7-OH, analogous to 7-OH formed from mitragynine) (FIG. 3B). Accordingly, 3-DM provides a significant advantage over mitragynine because it attenuates formation of the toxic metabolite DHM while having no effect on formation of the active metabolite 3-d-7-OH.

HLM Metabolite Formation. Pooled HLM from 50 adult male and female donors (XenoTech H0630, lot 1610016) were used. Microsomal incubations of mitragynine and 3-DM were carried out in 96-well plates in 5 aliquots of 40 μL each (one for each time point). Liver microsomal incubation medium contained PBS (100 mM, pH 7.4), MgCl2 (3.3 mM), NADPH (3 mM), glucose-6-phosphate (5.3 mM), glucose-6-phosphate dehydrogenase (0.67 units/mL) with 0.42 mg of liver microsomal protein per ml. Control incubations were performed replacing the NADPH-cofactor system with PBS. Test compounds (2 μM, final solvent concentration 1.6%) were incubated with microsomes at 37° C., shaking at 100 rpm. Incubations were performed in duplicate. Five time points over 40 minutes were analyzed. The reactions were stopped by adding 8 volumes of 90% acetonitrile-water to incubation aliquots, followed by protein sedimentation by centrifugation at 5500 rpm for 3 minutes. Supernatants were analyzed for parent compound remaining and metabolites DHM (both mitragynine and 3-DM incubations), 7-OH (mitragynine incubations), and 3-d-7-OH (3-DM incubations), using a fit-for-purpose liquid chromatography-tandem mass spectrometry (LC-MS/MS) method, with authentic samples of each analyte used for calibration and identification.

### Example 4. Attenuated Formation of 3-Dehydromitragynine in Brain Homogenate

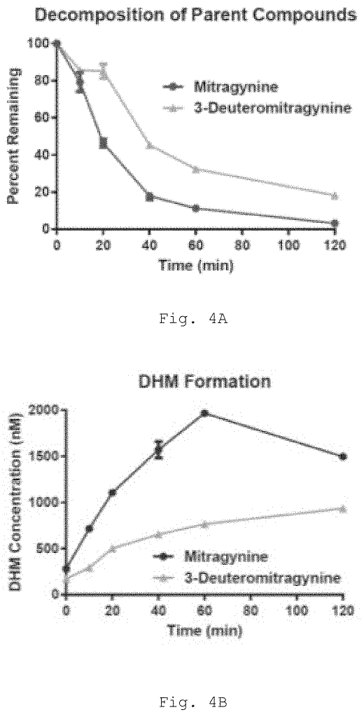

In mouse brain homogenate (MBH), mitragynine is unstable and decomposes to form 3-dehydromitragynine (DHM) as a major metabolite (FIG. 4). Deuteration of the 3 position of mitragynine, as in 3-deuteromitragynine (3-DM), slows the decomposition of mitragynine (FIG. 4A) and attenuates formation of DHM (FIG. 4B), via a kinetic isotope effect. Accordingly, 3-DM provides a significant advantage over mitragynine because it is both more stable in the brain and also attenuates formation of the toxic metabolite DHM directly in the brain.

MBH Preparation. Male BALB/c mice (12-14 weeks old) were housed in polypropylene cages with free access to standard commercial food pellets and tap water. Animals were sacrificed by cervical dislocation immediately prior to brain homogenate preparation. Brains from 10 mice were fragmented into small pieces and homogenized in ice-cold artificial cerebrospinal fluid solution (ACSF: 126 mM NaCl, 2.68 mM KCl, 1 mM Na₂HPO₄, 0.88 mM MgSO₄, 22 mM NaHCO₃, 1.45 mM CaCl₂, 10 mM HEPES, 11 mM D-glucose, pH 7.4) using a TH-01 OMNI homogenizer. Samples were centrifuged at 1500×g for 10 minutes. Supernatants were decanted and collected. Total protein concentration was determined by Bradford assay and equaled 17.7 mg/mL. The obtained brain homogenate was flash-frozen in liquid nitrogen. Aliquots were stored at −70° C. until use.

MBH Stability and Metabolite Formation. Brain homogenate incubations of mitragynine and 3-DM were carried out in 96-well plates in 6 aliquots of 40 μL each. The incubation medium consisted of artificial cerebrospinal fluid solution (ACSF: 126 mM NaCl, 2.68 mM KCl, 1 mM Na₂HPO₄, 0.88 mM MgSO₄, 22 mM NaHCO₃, 1.45 mM CaCl₂, 10 mM HEPES, 11 mM D-glucose, pH 7.4) with 2 mg of brain protein per mL. Test compounds (2 μM, final solvent concentration 1%) were incubated with brain homogenate at 37° C., shaking at 100 rpm. Incubations were performed in duplicate. Six time points over 120 minutes were analyzed. The reactions were stopped by adding 10 volumes of a 40% acetonitrile-40% methanol-20% water mixture to incubation aliquots, followed by protein sedimentation by centrifugation at 5500 rpm for 3 minutes. Supernatants were analyzed for parent compound remaining and DHM, using a fit-for-purpose liquid chromatography-tandem mass spectrometry (LC-MS/MS) method, with authentic samples of each analyte used for calibration and identification.

### Example 5. Attenuated Formation of 3-Dehydromitragynine in Mice (Pharmacokinetics)

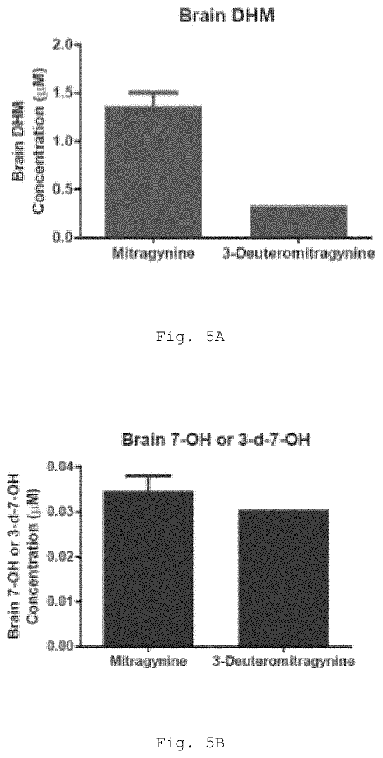

In mice, both 7-hydroxymitragynine (7-OH) and 3-dehydromitragynine (DHM) are formed as metabolites of mitragynine (FIG. 5) and both metabolites can be detected in the brain. Deuteration of the 3 position of mitragynine, as in 3-deuteromitragynine (3-DM), attenuates formation of DHM via a kinetic isotope effect and reduces the concentration of this compound observed in the brain (FIG. 5A). At the same time, deuteration at position 3 has no effect on oxidative metabolism at the 7 position to give 3-deutero-7-hydroxymitragynine (3-d-7-OH, analogous to 7-OH formed from mitragynine) (FIG. 5B). Accordingly, 3-DM provides a significant advantage over mitragynine because it attenuates formation of the toxic metabolite DHM while having no effect on formation of the active metabolite 3-d-7-OH.

Animals. This study was conducted using male 129S1 mice, 7 weeks of age, purchased from The Jackson Laboratory (Bar Harbor, Me.). Animals were housed in groups of five and allowed to acclimate for 1 week prior to testing. Mice had ad libitum access to food and water and were maintained on a 12-hour light/dark cycle. All testing was done in the light cycle.

Drugs. Mitragynine and 3-DM were dissolved in double-distilled H₂O containing 2 molar equivalents of acetic acid and 1.25% N-methyl-2-pyrrolidone (NMP). Drugs were administered subcutaneously at a volume of 10 mL/kg body weight and a dose of 10 mg/kg.

Pharmacokinetics. Animals were euthanized using cervical dislocation 15 minutes after drug administration. Immediately after sacrifice, whole brains were dissected out and stored at −80° C. for later analysis. After thawing, brains were homogenized in ice-cold water and protein was precipitated by treatment with 3:1 acetonitrile:MeOH followed by centrifugation at 13,500×g for 8 minutes. Supernatants were analyzed for parent compound remaining and metabolites DHM (both mitragynine and 3-DM treatment), 7-OH (mitragynine treatment), and 3-d-7-OH (3-DM treatment), using a fit-for-purpose liquid chromatography-tandem mass spectrometry (LC-MS/MS) method, with authentic samples of each analyte used for calibration and identification.

### Example 6. Attenuated Formation of 3-Dehydromitragynine in Simulated Gastric Fluid (SGF)

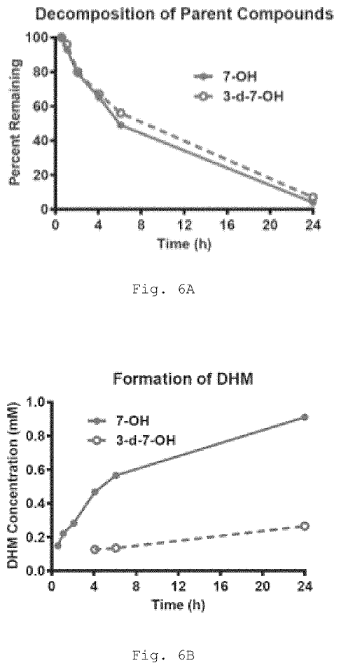

It is also found that 3-dehydromitragynine (OHM) is formed by dehydration and rearrangement of 7-hydroxymitragynine (7-OH) under protic acidic conditions in either aqueous or organic solvents. Accordingly, this conversion also occurs in contact with stomach acid (HCl) and the toxic metabolite DHM may thus form following the direct oral administration of 7-OH. However, deuteration of 7-hydroxymitragynine, as in 3-deutero-7-hydroxymitragynine (3-d-7-OH), attenuates this conversion via a kinetic isotope effect. This can be demonstrated by incubating 7-OH and 3-d-7-OH samples in simulated gastric fluid (SGF). Under these conditions, both 7-OH and 3-d-7-OH decompose at a similar rate (FIG. 6A), but the quantity of DHM formed from 3-d-7-OH is significantly reduced compared to that formed from 7-OH (FIG. 6B). Deuteration slows conversion of 3-d-7-OH to DHM and diverts the acid-catalyzed rearrangement toward other decomposition products (unknown NMR peaks of greater intensity were observed in the 3-d-7-OH incubations), these alternative pathways accounting for the identical rate of decomposition of the parent compounds. Accordingly, 3-d-7-OH provides a significant advantage over 7-OH because it permits oral administration with less risk of exposure to the toxic metabolite DHM.

SGF Incubations. Deuterated SGF (to permit direct NMR monitoring of the reactions) was prepared by combining NaCl (10 mg) and 37% DCl in D₂O (35 μL) and diluting up to a final volume of 5.0 mL with D₂O. Solutions of 7-OH and 3-d-7-OH in deuterated SGF were prepared at a concentration of 1.3 mg/mL containing N-methyl-2-pyrrolidone (NMP) as an internal standard (IS) at a concentration of 3.33 μL/mL and the reaction mixtures were left to stand at room temperature. NMR spectra were recorded on a Bruker 500 MHz instrument at the following time points: 35, 65, 125, 245, 365, and 1440 minutes (relative to time of solution mixing). Chemical shifts were referenced to the D₂O residual solvent peak at 4.79 ppm. Decomposition of parent compounds was quantified by the peak area ratio of a doublet at 6.63 ppm (corresponding to 7-OH and 3-d-7-OH) and a singlet at 2.84 ppm (corresponding to IS). These ratios, calculated at each time point, were normalized to the ratio determined at 35 minutes to obtain values as percent remaining (100% at 35 minutes). Concentration of DHM was determined at each time point by comparing the peak areas of a doublet at 6.70 ppm (corresponding to DHM) and a singlet at 2.84 ppm (corresponding to IS) and using the known concentration of internal standard (3.33 μL/mL=34.5 mM). The concentration of DHM formed from 3-d-7-OH was below the lower limit of quantitation of -0.1 mM at the 35-, 65-, and 125-minute time points.

### Example 7. Attenuated Formation of 3-Dehydromitragynine in Mice (Pharmacokinetics)

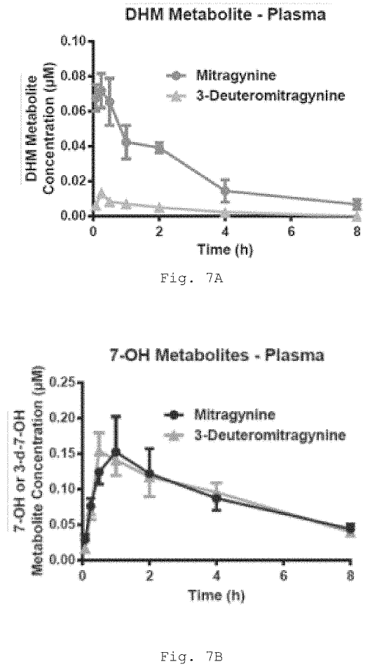

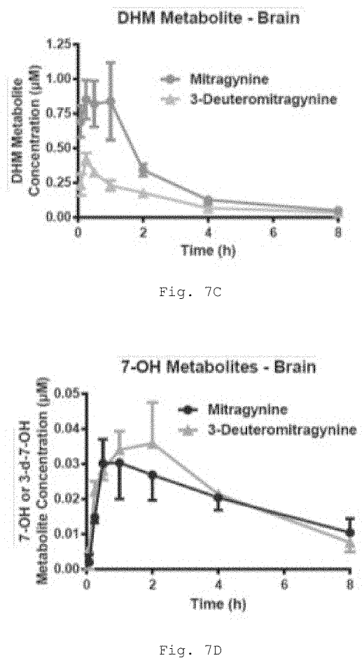

In mice, both 7-hydroxymitragynine (7-OH) and 3-dehydromitragynine (DHM) are formed as metabolites of mitragynine (FIG. 7) and both metabolites can be detected in the plasma and brain. Deuteration of the 3 position of mitragynine, as in 3-deuteromitragynine (3-DM), attenuates formation of DHM via a kinetic isotope effect and reduces the concentration of this compound observed in the plasma and brain (FIGS. 7A and 7C). At the same time, deuteration at position 3 has no effect on oxidative metabolism at the 7 position to give 3-deutero-7-hydroxymitragynine (3-d-7-OH, analogous to 7-OH formed from mitragynine) (FIGS. 7B and 7D). Accordingly, 3-DM provides a significant advantage over mitragynine because it attenuates formation of the toxic metabolite DHM while having no effect on formation of the active metabolite 3-d-7-OH.

Animals. Healthy male C₅₇BL/6 mice (8-12 weeks old) weighing between 19 to 28 g were procured from Global, India. Four mice were housed in each cage. Temperature and humidity were maintained at 22±3° C. and 30-70%, respectively and illumination was controlled on a 12-h light/dark cycle. Temperature and humidity were recorded by an auto-controlled data logger system. All animals were provided a laboratory rodent diet (Envigo Research Private Ltd, Hyderabad, India) and reverse osmosis water treated with ultraviolet light was provided ad libitum.

Drugs. Mitragynine and 3-DM were dissolved in normal saline made slightly acidic (˜pH 3) with 1M aqueous HCl. Drugs were administered subcutaneously at a volume of 10 mL/kg body weight and a dose of 10 mg/kg.

Pharmacokinetics. Blood samples (approximately 60 μL) were collected under light isoflurane anesthesia from the retro orbital plexus at 0.083, 0.25, 0.5, 1, 2, 4, 8 and 24 h (4 animals per time point). Plasma samples were separated by centrifugation of whole blood and stored below −70° C. until bioanalysis. Immediately after collection of blood, mice were euthanized and brain samples were collected at 0.083, 0.25, 0.5, 1, 2, 4, 8 and 24 h (4 animals per time point). Brain samples were homogenized using ice-cold phosphate buffered saline (pH 7.4) and homogenates were stored below −70° C. until analysis. Total homogenate volume was three times the tissue weight. Aliquots (25 μL) of each study sample (dilution factor applied for several samples) or spiked calibration standard were added to individual micro-centrifuge tubes followed by 100 μL of internal standard solution prepared in acetonitrile (Glipizide, 500 ng/mL; except for blank, where 100 μL of acetonitrile was added). Samples were vortexed for 5 minutes and then centrifuged for 5 minutes at a speed of 4000 rpm at 4° C. Following centrifugation, supernatants were analyzed for metabolites DHM (both mitragynine and 3-DM treatment) and 7-OH (mitragynine treatment) or 3-d-7-OH (3-DM treatment), using a fit-for-purpose liquid chromatography-tandem mass spectrometry (LC-MS/MS) method, with authentic samples of each analyte used for calibration and identification.

### Example 8. Identification of Metabolites of 3-Dehydromitragynine (M1, M4, and M6)

In mouse liver S9 fraction (MS9), three major metabolites of 3-dehydromitragynine (DHM), M1, M4, and M6, were identified by liquid chromatography-tandem mass spectrometry (LC-MS/MS) analysis (Scheme 3 and Table 1). Oxidative demethylation at the 9 position yields M4, which is followed by glucuronidation to give M1. Alternatively, demethylation occurs on the acrylate moiety to give M6 (note—exact site of demethylation not identified conclusively, could occur at either the ester or enol ether). Other minor metabolites (M2, M3, and M5) were also identified (Table 1).

TABLE 1
Metabolites of DHM detected in positive ion mode in MS9 after
60-min incubation.

| | | | | | | Found in | |
|---|---|---|---|---|---|---|---|
| | | Expected | Mass | MRM | | mouse | |
| | Metabolic | m/z, Da | shift, | transition | RT, | S9 liver | % |
| Reference | transformation | [M + 1] | Δm, Da | Q→Q3 | min | fraction | Area |
| DHM | Parent | 397.3 | 0 | 397.3/227.1 | 18.4 | + | 20 |
| M1 | Demethylation + | 559.3 | 162 | 559.3/383.3 | 9.7 | + | 10 |
| | Glucuronidation | | | | | | |
| M2 | Di-Demethylation | 369.3 | −28 | 369.3/213.1 | 10.5 | + | 1.5 |
| M3 | Oxidation | 413.3 | 16 | 413.3/227.1 | 13.0 | + | 0.7 |
| M4 | Demethylation | 383.3 | −14 | 383.3/213.1 | 13.3 | + | 23 |
| M5 | Oxidation + | 589.3 | 192 | 589.3/413.3 | 15.0 | + | 0.9 |
| | Glucuronidation | | | | | | |
| M6 | Demethylation | 383.3 | −14 | 383.3/227.1 | 15.9 | + | 41 |

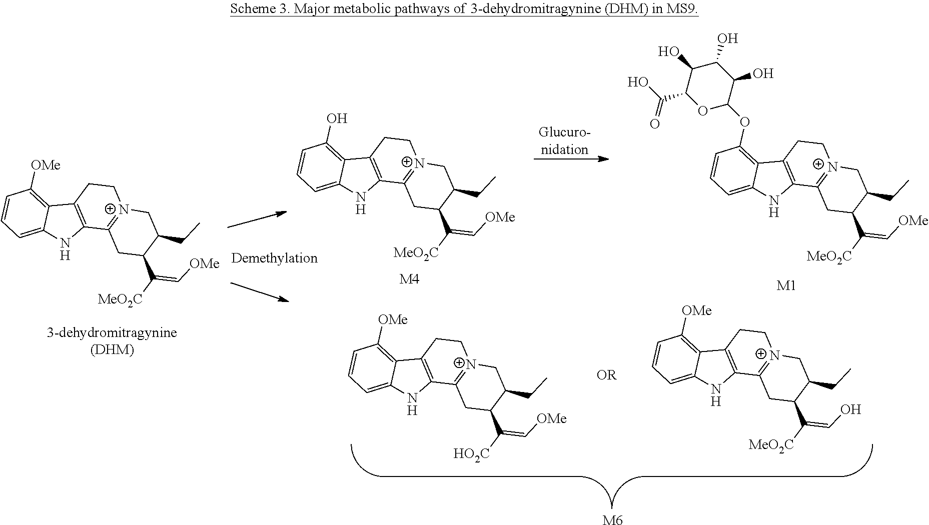

Analytical System. The metabolic profiling of DHM in mouse liver S9 fraction was performed using high performance liquid chromatography-tandem mass spectrometry (HPLC-MS/MS). A Shimadzu HPLC system comprising 2 isocratic pumps (LC-10ADvp), an autosampler (SIL-30ACMP), a system controller (CBM-20A), a high-pressure switching valve (FCV-20AH6), and a degasser (DGU-14A) was used for separations. Mass spectrometric analyses were performed using an API 4000 QTRAP mass spectrometer from Applied Biosystems/MDS Sciex (AB Sciex) with Turbo V ion source and TurboIonspray interface. Data acquisition and system control was performed using Analyst 1.6.3 software from AB Sciex.

Liver S9 Incubations. Liver S9 fraction pooled from male CD-1 mice was used. Incubations were carried out in 1.1 mL microtubes (in 96-well format plate) in aliquots of 40 μL each (2 for each time point; 0, 6, 16, 30, and 60 min). The S9 incubation medium contained phosphate buffer (100 mM, pH 7.4), MgCl₂ (3.3 mM), NADPH (3 mM), glucose-6-phosphate (5.3 mM), glucose-6-phosphate dehydrogenase (0.67 units/mL), UDPGA (2.5 mM), PAPS (0.3 mM), and reduced glutathione (GSH, 2 mM), with 2 mg of S9 protein per mL. Control incubations were performed replacing the cofactor system with phosphate buffer. Test compound DHM (10 μM, final solvent concentration 1.6%) was incubated with S9 at 37° C., shaking at 100 rpm. Incubations were performed in duplicate. Five time points over 60 minutes (0, 6, 16, 30, and 60 min) were analyzed. The reactions were stopped by adding 6 volumes of 90% acetonitrile-water to incubation aliquots followed by protein sedimentation by centrifugation at 5500 rpm for 3 minutes. Supernatants (two time points, 0 and 60 min) were analyzed for remaining parent compound and putative metabolites by HPLC-MS/MS.

General Strategy for Identification of Metabolites. The approach to metabolite profiling integrated multiple reaction monitoring of predicted metabolites with information dependent acquisition (MRM-IDA) using a hybrid triple quadrupole linear ion trap mass spectrometer. The strategy for metabolite identification integrated the following steps:

1. Determination of parent compound fragmentation pathways by analysis of MS/MS product ion spectra (product ion scan, MS2). The assignment of MS/MS product ions with specific fragments of the molecule was performed using ACD/Labs MS Fragmenter software.

2. Multiple MRM-IDA methods were created using LightSight software (AB Sciex). LightSight comprises a comprehensive database of all classical metabolic biotransformations, both phases I and II of metabolism, allowing it to create MRM methods for a full set of predicted metabolites. The methods comprised the survey MRM-IDA scans for parent compound and metabolites linked to information dependent enhanced product ion (EPI) scans.

3. The identification of metabolites found by the MRM-IDA experiment was performed by analysis of spectra obtained by enhanced product ion (EPI) scans. Interpretation of the MS/MS product spectra of all metabolites and comparison with the parent compound demonstrated the mass shifts in specific fragments and showed the substructures that were metabolized.

### Example 9. Attenuated Formation of M1, M4, and M6 Metabolites in Mouse Liver S9 Fraction

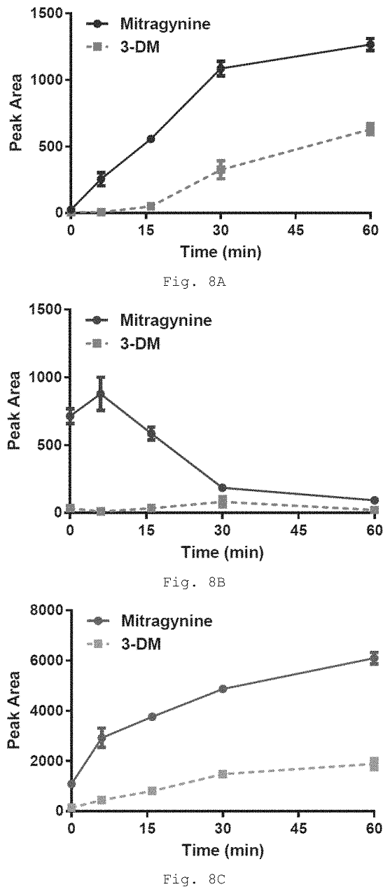

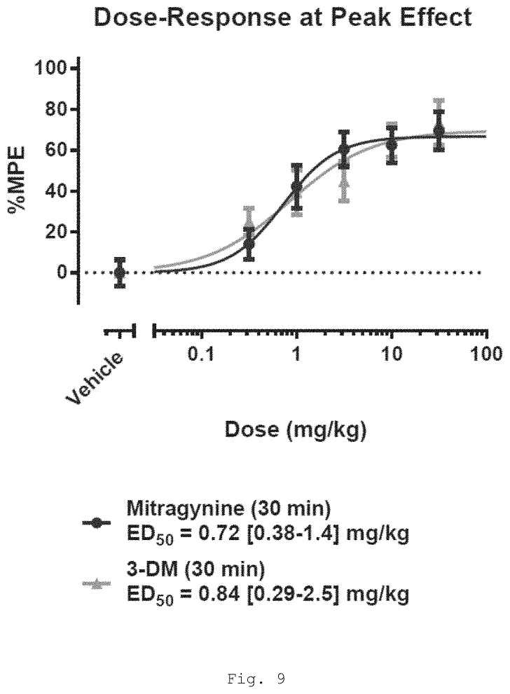

In mouse liver S9 fraction (MS9), metabolites M1, M4, and M6 are formed as downstream metabolites of 3-dehydromitragynine (DHM) (FIG. 8, see also Example 8). Deuteration of the 3 position of mitragynine, as in 3-deuteromitragynine (3-DM), attenuates formation of M1, M4, and M6 by slowing formation of their parent compound DHM via a kinetic isotope effect (FIG. 8). This attenuated formation of downstream metabolites of DHM (M1, M4, and M6) provides further evidence for the attenuation of DHM formation by 3-DM as compared to non-deuterated mitragynine.

MS9 Metabolite Formation. Liver S9 fraction pooled from male CD-1 mice was used. Incubations were carried out in 96-well plates in 5 aliquots of 40 μL each (one for each time point). The S9 incubation medium contained phosphate buffer (100 mM, pH 7.4), MgCl₂ (3.3 mM), NADPH (3 mM), glucose-6-phosphate (5.3 mM), glucose-6-phosphate dehydrogenase (0.67 units/mL), UDPGA (2.5 mM), PAPS (0.3 mM), and reduced glutathione (2 mM), with 2 mg of S9 protein per mL. Control incubations were performed replacing the cofactor system with phosphate buffer. Test compounds (2 μM, final solvent concentration 1.6%) were incubated with S9 at 37° C., shaking at 100 rpm. Incubations were performed in duplicate. Five time points over 60 minutes were analyzed. The reactions were stopped by adding 8 volumes of 90% acetonitrile-water to incubation aliquots, followed by protein sedimentation by centrifugation at 5500 rpm for 3 minutes. Supernatants were analyzed for remaining parent compound and metabolites by liquid chromatography-tandem mass spectrometry (LC-MS/MS).

### Example 10. 3-Alkylmitragynine Derivatives

3-Methylmitragynine and other 3-alkylmitragynine derivatives are prepared (Scheme 4) by treatment of 3-dehydromitragynine (iminium form) with appropriate organometallic alkylating reagents, for example, according to published procedures (Barteselli, A. et al. 2015; Nakagawa, M. et al. 1990). Like deuteration, alkylation of the 3-position is expected to also attenuate metabolic conversion to the toxic metabolite 3-dehydromitragynine.

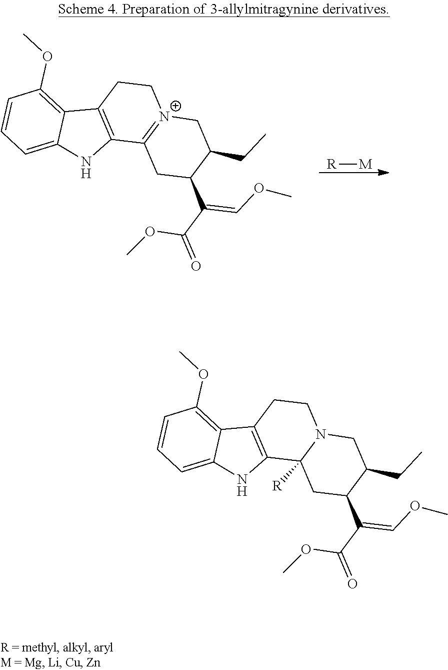

### Example 11. 3-Deuterospeciociliatine and 3,14-Dideuterospeciociliatine Derivatives

3-Deuterospeciociliatine is prepared (Scheme 5) by enantioselective hydrogenation of 3-dehydromitragynine. For example, following reported procedures, 3-dehydromitragynine (iminium form) is treated with a mixture of Noyori's catalyst, silver hexafluoroantimonate (V), cetrimonium bromide, and deuterated sodium formate in D₂O to give the desired product (Evanno, L. et al. 2009; Piemontesi, C. et al. 2016).

Similar procedures are used to prepare 3,14-dideuterospeciociliatine (Scheme 5) by first treating 3-dehydromitragynine (iminium form) with base to generate the corresponding enamine form (3,14-dehydromitragynine), followed by treatment under enantioselective hydrogenation conditions to give the desired product.

In both compounds, deuteration of the 3-position attenuates metabolic conversion to the toxic metabolite 3-dehydromitragynine.

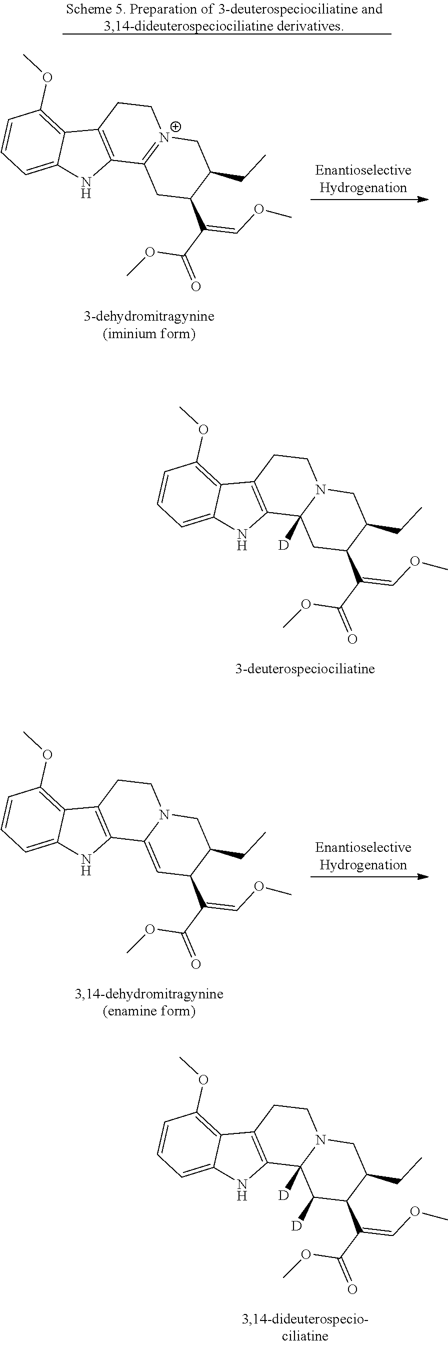 [SMILES: CC[C@@H]1C[N+]2=C(C[C@@H]1/C(=C\OC)C(=O)OC)c1[nH]c3cccc(OC)c3c1CC2 ; [2H][C@]12C[C@H](/C(=C\OC)C(=O)OC)[C@H](CC)CN1CCc1c2[nH]c2cccc(OC)c12 ; CC[C@@H]1CN2CCc3c([nH]c4cccc(OC)c34)C2=C[C@@H]1/C(=C\OC)C(=O)OC ; [2H][C@@H]1[C@H](/C(=C\OC)C(=O)OC)[C@H](CC)CN2CCc3c([nH]c4cccc(OC)c34)[C@@]12[2H]]

### Example 12. Deuterated Corynantheidine Derivatives

Deuterated derivatives of corynantheidine are prepared according to the procedures shown in Schemes 6A-B. In such compounds, deuteration of the 3-position attenuates metabolic or acid-mediated (in the case of 7-hydroxy derivatives) conversion to the corresponding toxic metabolites 3-dehydrocorynantheidine or 3-dehydro-9-hydroxycorynantheidine.

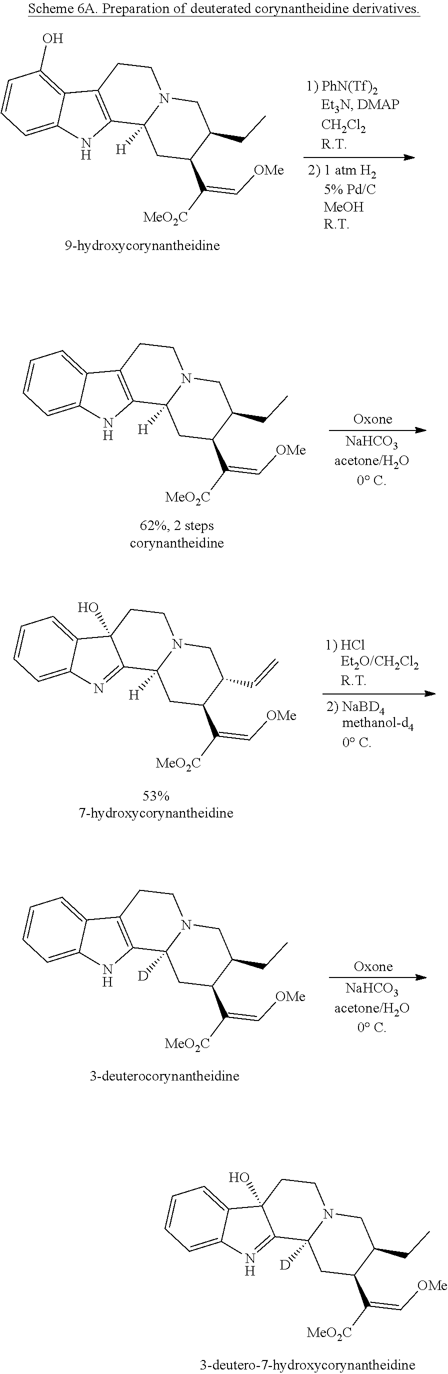 [SMILES: CC[C@@H]1CN2CCc3c([nH]c4cccc(O)c34)[C@@H]2C[C@@H]1/C(=C\OC)C(=O)OC ; CC[C@@H]1CN2CCc3c([nH]c4ccccc34)[C@@H]2C[C@@H]1/C(=C\OC)C(=O)OC ; C=C[C@H]1CN2CC[C@@]3(O)C(=Nc4ccccc43)[C@@H]2C[C@@H]1/C(=C\OC)C(=O)OC ; [2H][C@@]12C[C@H](/C(=C\OC)C(=O)OC)[C@H](CC)CN1CCc1c2[nH]c2ccccc12 ; [2H][C@@]12C[C@H](/C(=C\OC)C(=O)OC)[C@H](CC)CN1CC[C@@]1(O)C2=Nc2ccccc21]

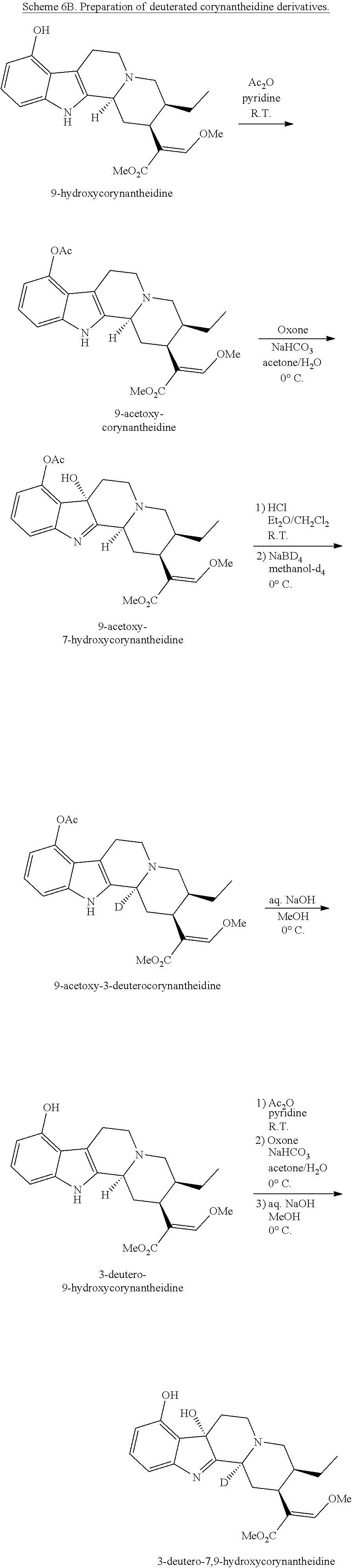 [SMILES: CC[C@@H]1CN2CCc3c([nH]c4cccc(O)c34)[C@@H]2C[C@@H]1/C(=C\OC)C(=O)OC ; CC[C@@H]1CN2CCc3c([nH]c4cccc(OC(C)=O)c34)[C@@H]2C[C@@H]1/C(=C\OC)C(=O)OC ; CC[C@@H]1CN2CC[C@@]3(O)C(=Nc4cccc(OC(C)=O)c43)[C@@H]2C[C@@H]1/C(=C\OC)C(=O)OC ; [2H][C@@]12C[C@H](/C(=C\OC)C(=O)OC)[C@H](CC)CN1CCc1c2[nH]c2cccc(OC(C)=O)c12 ; CC[C@@H]1CN2CCc3c([nH]c4cccc(O)c34)[C@@H]2C[C@@H]1/C(=C\OC)C(=O)OC ; [2H][C@@]12C[C@H](/C(=C\OC)C(=O)OC)[C@H](CC)CN1CC[C@@]1(O)C2=Nc2cccc(O)c21]

9-Hydroxycorynantheidine. 9-Hydroxycorynantheidine was obtained by demethylation of mitragynine as previously described (Kruegel et al., 2016). Spectral and physical properties were in agreement with those previously reported (Kruegel et al. 2016).

Corynantheidine. To a solution of 9-hydroxycorynantheidine (2.00 g, 5.20 mmol) in anhydrous CH₂Cl₂ (139 mL) under argon at room temperature was added 4-(dimethylamino)pyridine (127 mg, 1.04 mmol), Et₃N (1.45 mL, 1.05 g, 10.40 mmol), and N-phenyl-bis(trifluoromethanesulfonimide) (2.42 g, 6.76 mmol), and the resulting brown solution was left to stir at room temperature. After 2 h, the reaction mixture was concentrated in vacuo to give a sticky dark-brown glass (5.27 g). This material was purified directly by column chromatography (8:2 hexanes:EtOAc, 3 column volumes 7:3 hexanes:EtOAc, 2 column volumes 1:1 hexanes:EtOAc, 1 column volume) to give the triflate intermediate containing impurities as a very pale-yellow foam (2.49 g). A quantity (2.45 g) of this material was combined with 5% Pd on carbon (2.45 g), MeOH (47.5 mL) was added, and the mixture was stirred at room temperature under 1 atm H₂ for 2 h. The mixture was then filtered through celite, the filter cake was washed with MeOH (3×50 mL), and the combined filtrates concentrated in vacuo to give the crude product as a pale-yellow foam (2.12 g). This material was purified by column chromatography (8:2 hexanes:EtOAc+2% Et₃N, 3 column volumes→7:3 hexanes:EtOAc+2% Et₃N, 3 column volumes) to give pure corynantheidine as an amorphous off-white solid (1.17 g, 62% over 2 steps). ¹H NMR (500 MHz, CDCl₃) δ 7.76 (br s, 1H), 7.47 (d, J=7.6 Hz, 1H), 7.44 (s, 1H), 7.30 (d, J=7.9 Hz, 1H), 7.15-7.04 (m, 2H), 3.73 (s, 3H), 3.72 (s, 3H), 3.19 (dd, J=11.4, 2.4 Hz, 1H), 3.09-2.93 (m, 4H), 2.74-2.66 (m, 1H), 2.62-2.46 (m, 3H), 1.88-1.73 (m, 2H), 1.68-1.60 (m, 1H), 1.28-1.16 (m, 1H), 0.88 (t, J=7.4 Hz, 3H); ¹³C NMR (126 MHz, CDCl₃) δ 169.4, 160.7, 136.0, 135.7, 127.7, 121.3, 119.4, 118.2, 111.6, 110.8, 108.2, 61.7, 61.4, 57.9, 53.6, 51.5, 40.8, 40.1, 30.0, 22.0, 19.2, 13.0.

7-Hydroxycorynantheidine. To a solution of corynantheidine (479 mg, 1.30 mmol) in acetone (39 mL) was added saturated aqueous NaHCO₃ (26 mL) and the mixture was cooled to 0° C. A solution of Oxone monopersulfate (2KHSO₅.KHSO₄.K₂SO₄; 800 mg, 1.30 mmol) in water (13 mL) was then pre-cooled to 0° C. and added in 20 approximately equal portions over 20 minutes. At the end of the addition, the reaction mixture was diluted with water (100 mL) and extracted with EtOAc (3×50 mL). The combined organics were then washed with brine (50 mL), dried over Na₂SO₄, and concentrated in vacuo to give the crude product (0.48 g). This material was purified by column chromatography (7:3 hexanes:EtOAc+2% Et₃N, 3 column volumes→6:4 hexanes:EtOAc+2% Et₃N, 3 column volumes) to give 7-hydroxycorynantheidine as an amorphous yellow solid (264 mg, 53%). ¹H NMR (400 MHz, Methanol-d₄) δ 7.56 (s, 1H), 7.53 (dt, J=7.7, 0.9 Hz, 1H), 7.45 (ddd, J=7.3, 1.3, 0.7 Hz, 1H), 7.37 (td, J=7.6, 1.3 Hz, 1H), 7.26 (td, J=7.4, 1.0 Hz, 1H), 3.86 (s, 3H), 3.70 (s, 3H), 3.13 (ddd, J=15.4, 11.5, 2.2 Hz, 2H), 3.04 (dt, J=13.6, 3.4 Hz, 1H), 2.91-2.78 (m, 2H), 2.65 (ddd, J=11.8, 4.5, 2.3 Hz, 1H), 2.52-2.45 (m, 1H), 2.41 (dt, J=14.0, 2.4 Hz, 1H), 1.87-1.80 (m, 1H), 1.76-1.58 (m, 2H), 1.53 (ddd, J=13.9, 12.9, 4.4 Hz, 1H), 1.38-1.24 (m, 1H), 0.85 (t, J 7.2 Hz, 3H); ¹³C NMR (101 MHz, Methanol-d₄) δ 186.7, 170.8, 162.5, 154.0, 143.1, 130.4, 127.5, 123.5, 121.5, 112.1, 81.3, 62.9, 62.3, 59.2, 51.8, 51.5, 42.1, 40.6, 37.6, 27.3, 20.1, 13.2.

### Example 13. Opioid Receptor Binding of Deuterated Compounds

Deuterium-enriched compounds of the present invention are tested for binding affinity at opioid receptors (MOR, KOR, and/or DOR) using radioligand displacement experiments. The binding affinities of the deuterium-enriched compounds are substantially similar to those of their non-deuterated counterparts.

### Example 14. Opioid Receptor Functional Activity of Deuterated Compounds

Deuterium-enriched compounds of the present invention are tested in vitro for functional activity (either agonist or antagonist) at opioid receptors (MOR, KOR, and/or DOR). The functional activities of the deuterium-enriched compounds are substantially similar, in both potency and type (agonist or antagonist), to those of their non-deuterated counterparts.

### Example 15. Analgesic Activity of 3-Deuteromitragynine in Rats

Mitragynine and 3-deuteromitragynine (3-DM) were tested in the tail-flick assay in rats. Both compounds exhibited a dose-dependent analgesic effect with similar potency and maximal efficacy (FIG. 9).

Animals. Male Sprague-Dawley rats, aged 7-8 weeks, were used in experiments. Animals were housed under controlled temperatures and 12-hour light/dark cycles (lights on at 06:00-18:00 h), with ad libitum food and water. The study was approved by the Institutional Animal Care and Use Committee (IACUC) at WuXi AppTec (Shanghai). WuXi's animal facilities and IACUC are fully accredited by the Association for Assessment and Accreditation of Laboratory Animal Care International (AAALAC). All efforts were made to minimize animal suffering.

Drugs and Drug Administration. Drugs were prepared as described above and administered by oral gavage at a volume of 5 mL/kg in de-ionized water acidified with acetic acid.

Tail-Flick. Analgesic activity was assessed in the tail-flick assay 30 minutes (peak effect) after administration of vehicle and each dose of drug (n=8 per treatment). Animals were tested for their latency to withdraw their tails from a hot water bath maintained at 50° C. with a room temperature of 22° C. A cutoff time of 7 seconds was used to prevent tissue damage. Data were analyzed as percent maximal effect, % MPE, which was calculated according to the formula: % MPE=[(observed latency−vehicle latency)/(maximal latency−vehicle latency)]×100. Dose-response curves were fit by nonlinear regression (GraphPad Prism, La Jolla, Calif.).

### Example 16. Administration of MOR Agonists

An amount of a composition comprising any one of the following compounds:

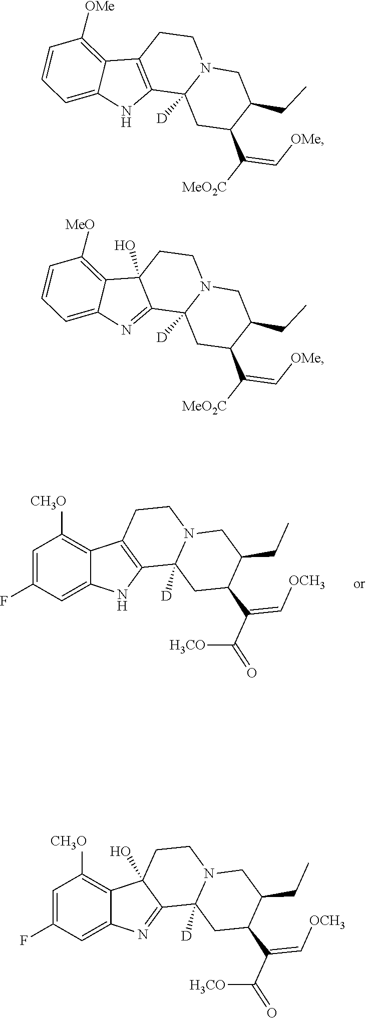

is administered to a subject afflicted with pain, a depressive disorder, an anxiety disorder, a mood disorder, borderline personality disorder, a substance use disorder, opioid use disorder or opioid withdrawal symptoms. The amount of the compound is effective to treat the subject. In these structures, D represents a deuterium-enriched site.

The non-deuterated analogs of the above compounds were previously shown to be active as MOR agonists and, thus, useful as treatments for pain, mood disorders, depressive disorders, anxiety disorders, and opioid use disorder (Kruegel et al. 2016; WO/2017/165738 A1). Analogous examples are repeated with deuterium-enriched compounds.

The effects are substantially similar. However, formation of toxic metabolites is significantly attenuated.

### Example 17. Administration of MOR Antagonists

An amount of a composition comprising any one of the following compounds:

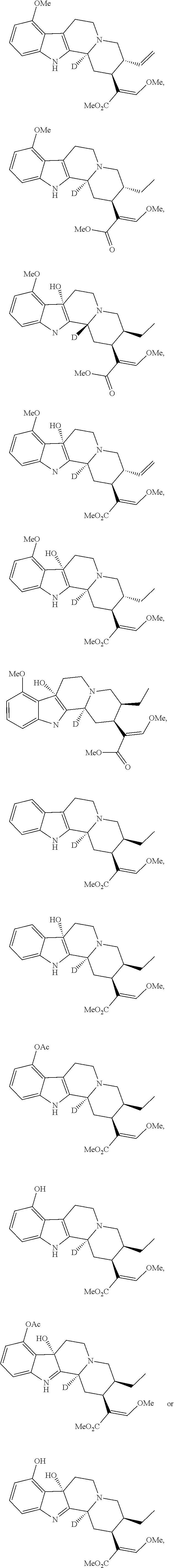

is administered to a subject afflicted with a depressive disorder, a mood disorder, an anxiety disorder, borderline personality disorder, a substance use disorder or opioid use disorder. The amount of the compound is effective to treat the subject. In these structures, D represents a deuterium-enriched site.

An amount of a composition comprising any one of the following compounds:

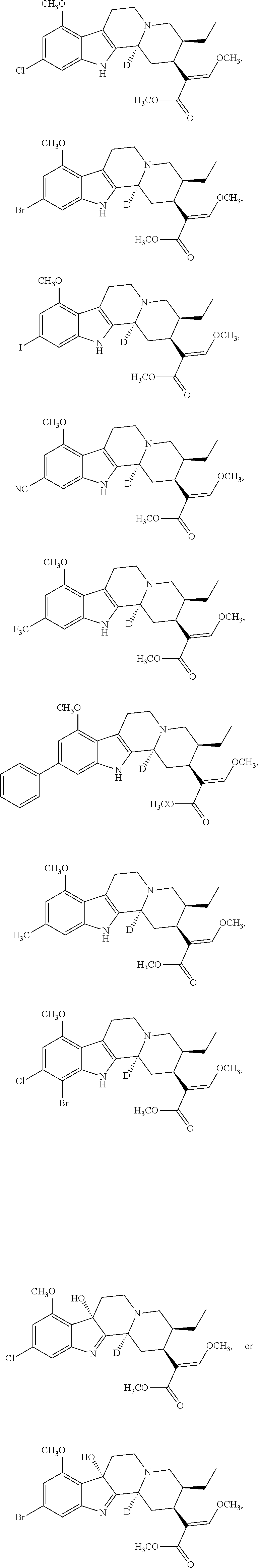

is administered to a subject afflicted with a depressive disorder, a mood disorder, an anxiety disorder, borderline personality disorder,
a substance use disorder or opioid use disorder. The amount of the compound is effective to treat the subject. In these structures, D represents a deuterium-enriched site.

### Example 18. Combinations with NMDA Receptor Antagonists

Antagonists of the N-methyl-D-aspartate receptor (NMDAR) are known to potentiate the beneficial effects of opioid receptor agonists in the treatment of pain and to prevent the development of tolerance to those effects (Trujillo, K. A. et al. 1994; Mao, J. et al. 1996). NMDAR antagonists are also known to be effective in the treatment of depression (Murrough, J. W. et al. 2013). Therefore, pharmaceutical compositions of the compounds disclosed herein, combined with NMDAR antagonists, may be useful in the treatment of pain, anxiety disorders or mood disorders with increased efficacy and/or slower development of tolerance. Alternatively, the opioid modulator and NMDAR antagonist may be dosed separately, as a novel method for treating pain, anxiety disorders or mood disorders.

### Non-Limiting Examples of NMDA Receptor Antagonists:

Dextromorphinans—dextromethorphan, dextrorphan, dextrallorphan

Adamantanes—memantine, amantadine, rimantadine, nitromemantine (YQW -36)

Arylcyclohexylamines—ketamine (and its analogs, e.g. tiletamine), phencyclidine (and its analogs, e.g. tenocyclidine, eticyclidine, rolicyclidine), methoxetamine (and its analogs), gacyclidine (GK-11)

Miscellaneous—neramexane, lanicemine (AZD6765), diphenidine, dizocilpine (MK-801), 8a-phenyldecahydroquinoline (8A-PDHQ), remacemide, ifenprodil, traxoprodil (CP-101,606), eliprodil (SL-82.0715), etoxadrol (CL-1848C), dexoxadrol, WMS-2539, NEFA, delucemine (NPS-1506), aptiganel (Cerestat; CNS-1102), midafotel (CPPene; SDZ EAA 494), dexanabinol (HU-211 or ETS2101), selfotel (CGS-19755), 7-chlorokynurenic acid (7-CKA), 5,7-dichlorokynurenic acid (5,7-DCKA), L-683344, L-689560, L-701324, GV150526A, GV196771A, CERC-301 (formerly MK-0657), atomoxetine, LY-235959, CGP 61594, CGP 37849, CGP 40116 (active enantiomer of CGP 37849), LY-233536, PEAQX (NVP-AAM077), ibogaine, noribogaine, Ro 25-6981, GW468816, EVT-101, indantadol, perzinfotel (EAA-090), SSR240600, 2-MDP (U-23807A), AP-7

### Example 19. Combinations with NMDA Receptor Partial Agonists

Weak partial agonists of NMDAR are also known (Moskal, J. R. et al. 2005), and may be expected to produce beneficial or synergistic effects similar to an antagonist when intrinsic glutamate signaling activity is high or over-activated. Therefore, pharmaceutical compositions of the novel compounds disclosed herein, combined with NMDAR partial agonists, may be useful in the treatment of pain, anxiety disorders or mood disorders with increased efficacy and/or slower development of tolerance. Alternatively, the opioid modulator and NMDAR partial agonist may be dosed separately, as a novel method for treating pain, anxiety disorders or mood disorders.

### Non-Limiting Examples of NMDA Receptor Partial Agonists:

NRX-1074, rapastinel (GLYX-13)

### Example 20. Combinations with Neurokinin 1 Receptor Antagonists

Antagonists of the neurokinin 1 receptor (NK-1) are known to modulate the effects of opioid agonists, specifically in reward and self-administration protocols. More specifically, NK-1 antagonists attenuate opioid reward and self-administration in animal models (Robinson, J. E. et al. 2012). NK-1 antagonists are also known to be effective in the treatment of depression (Kramer, M. S. et al. 2004). Therefore, pharmaceutical compositions of the novel compounds disclosed herein, combined with NK-1 antagonists, may be useful in the treatment of pain, anxiety disorders or mood disorders with increased efficacy and/or less potential for abuse. Alternatively, the opioid modulator and NK-1 antagonist may be dosed separately, as a novel method for treating pain, anxiety disorders or mood disorders.

### Non-Limiting Examples of Neurokinin 1 Receptor Antagonists:

aprepitant, fosaprepitant, casopitant, maropitant, vestipitant, vofopitant, lanepitant, orvepitant, ezlopitant, netupitant, rolapitant, L-733060, L-703606, L-759274, L-822429, L-760735, L-741671, L-742694, 1-732138, CP-122721, RPR-100893, CP-96345, CP-99994, TAK-637, T-2328, CJ-11974, RP 67580, NKP608, VPD-737, GR 205171, LY686017, AV608, SR140333B, SSR240600C, FK 888, GR 82334

### Example 21. Combinations with Neurokinin 2 Receptor Antagonists

Antagonists of the neurokinin 2 receptor (NK-2) are known to show antidepressant effects and to synergize with tricyclic antidepressants (Overstreet, D. H. et al. 2010). Therefore, pharmaceutical compositions of the novel compounds disclosed herein, combined with NK-2 antagonists, may be useful in the treatment of anxiety disorders or mood disorders with increased efficacy. Alternatively, the opioid modulator and NK-2 antagonist may be dosed separately, as a novel method for treating anxiety disorders or mood disorders.

### Non-Limiting Examples of Neurokinin 2 Receptor Antagonists:

saredutant, ibodutant, nepadutant, GR-159897, MEN-10376

### Example 22. Combinations with Neurokinin 3 Receptor Antagonists

Antagonists of the neurokinin 3 receptor (NK-3) are known to show antidepressant effects (Salome, et al. 2006). Further, the actions of NK-3 modulators show a dependency on the opioid receptor system (Panocka, I. et al. 2001). Therefore, pharmaceutical compositions of the novel compounds disclosed herein, combined with NK-3 antagonists, may be useful in the treatment of anxiety disorders or mood disorders with increased efficacy. Alternatively, the opioid modulator and NK-3 antagonist may be dosed separately, as a novel method for treating anxiety disorders or mood disorders.

Non-Limiting Examples of Neurokinin 3 Receptor Antagonists:

osanetant, talnetant, SB-222200, SB-218795

### Example 23. Combinations with DOR Agonists

DOR Agonists have also been shown to elicit antidepressant and anxiolytic effects (Saitoh, A. et al. 2004; Torregrossa, et al. 2005; Jutkiewicz, E. M. 2006) and are analgesic (Vanderah, T. W. 2010; Peppin, J. F. and Raffa, R. B. 2015). They have also been shown to reverse the respiratory depression induced by MOR agonists (Su, Y-F. et al. 1998). Therefore, pharmaceutical compositions of the novel compounds disclosed herein, combined with DOR agonists, may be useful in the treatment of pain, anxiety disorders, or mood disorders with increased efficacy or reduced side effects. Alternatively, the opioid modulator and DOR agonist may be dosed separately, as a novel method for treating pain, anxiety disorders or mood disorders.

### Non-Limiting Examples of DOR Agonists:

tianeptine, (+)BW373U86, SNC-80, SNC-121, SNC-162, DPI-287, DPI-3290, DPI-221, TAN-67, KN-127, AZD2327, JNJ-20788560, NIH11082, RWJ-394674, ADL5747, ADL5859, UFP-512, AR-M100390, SB-235863, 7-spiroindanyloxymorphone.

### Example 24. Combinations with Naloxone

Naloxone is a MOR antagonist that is effective in blockading all behavioral effects induced by classical MOR agonists and is the standard treatment for opioid overdose. It is highly bioavailable by parenteral routes of administration but not by the oral route (Smith, K. et al. 2012). Accordingly, pharmaceutical compositions containing mixtures of a MOR agonist and naloxone remain effective agonists when given by the oral route, but the naloxone component inhibits the effects of the MOR agonist component when the mixture is administered parenterally. Thus, addition of naloxone to pharmaceutical compositions containing MOR agonists is useful for preventing their misuse or abuse by parenteral routes of administration. Therefore, pharmaceutical compositions of the compounds of the present invention, combined with naloxone, may be useful in providing the therapeutic benefits of the compounds of the present invention while having diminished potential for abuse.

### Example 25. Combinations with SSRIs or SNRIs

Selective serotonin reuptake inhibitors (SSRIs) and serotonin-norepinephrine reuptake inhibitors (SNRIs) are the standard of care for many depressive disorders, mood disorders, and anxiety disorders (Thase, M. E. 2008; Vaswani, M. et al. 2003). They are also useful in the treatment of chronic pain (Marks, D. M. et al. 2009). Therefore, pharmaceutical compositions of the compounds of the present invention, combined with SSRIs or SNRIs, are useful in the treatment of depressive disorders, mood disorders, borderline personality disorder, anxiety disorders, or pain with increased efficacy compared to the compounds of the present invention alone. Alternatively, the opioid modulator and SSRI or SNRI may be dosed separately, as a novel method for treating the conditions described above. Further, the compound of the present invention may be used as an add-on therapy to enhance the efficacy of preexisting SSRI or SNRI therapy for the conditions described above.

Non-Limiting Examples of SSRIs: citalopram, escitalopram, fluoxetine, fluvoxamine, paroxetine, sertraline, dapoxetine

Non-Limiting Examples of SNRIs: venlafaxine, desvenlafaxine

### Example 26. Combinations with Methylnaltrexone

Constipation is a frequent, unpleasant side effect of MOR agonists resulting from inhibition of intestinal smooth muscle contractions via activation of MORs located in this tissue. Methylnaltrexone (Relistor) is a clinically approved quaternary ammonium salt of the opioid receptor antagonist naltrexone that does not cross the blood brain barrier. Accordingly, this compound is capable of inhibiting MORs in the gastrointestinal tract and preventing opioid-induced constipation while avoiding simultaneous inhibition of centrally mediated therapeutic effects. Therefore, pharmaceutical compositions of the compounds of the present invention, combined with methylnaltrexone, are useful in the treatment of depressive disorders, mood disorders, borderline personality disorder, pain, opioid addiction, or opioid withdrawal symptoms with reduced constipation compared to the compounds of the present invention alone. Alternatively, the opioid modulator and methylnaltrexone may be dosed separately, as a novel method for treating the conditions described above with less constipation.

### Example 27. Treatment of Opioid Use Disorder

There is substantial human data suggesting the clinical efficacy of kratom leaf and/or its extracts in treating opioid withdrawal symptoms or opioid use disorder (Grundmann, O. 2017; Swogger, M. T. et al. 2015; Pain News Network; Smith, K. E. and Lawson, T. 2017). Further, in rats, mitragynine treatment attenuates later self-administration of opioid agonists, including heroin and morphine (Hemby, S. E. et al. 2018; Yue, K. et al. 2018). Accordingly, mitragynine and related compounds which act on mu-opioid receptors, and thus also the analogous deuterated compounds of this invention, are useful as treatments for opioid withdrawal and opioid use disorder.

### Example 28. Attenuated Formation of 3-Dehydromitragynine in Plasma

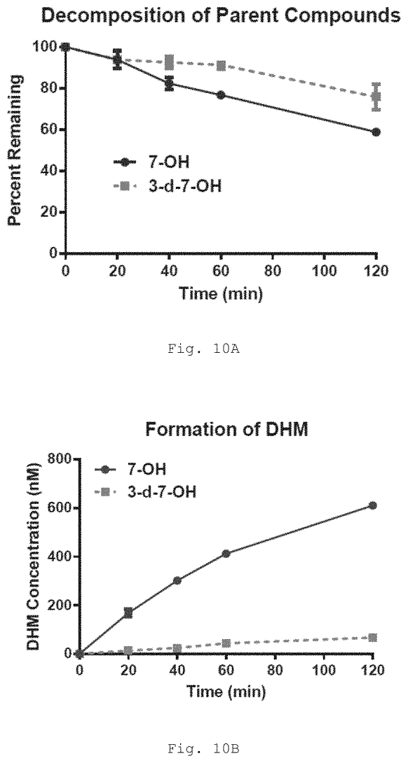

In dog plasma (DP), 7-hydroxymitragynine (7-OH) is unstable and decomposes to form 3-dehydromitragynine (DHM) (FIG. 10). Deuteration of the 3 position of 7-OH, as in 3-deutero-7-hydroxymitragynine (3-d-7-OH), slows the decomposition of mitragynine (FIG. 10A) and attenuates formation of DHM (FIG. 10B), via a kinetic isotope effect. Accordingly, 3-d-7-OH provides a significant advantage over 7-OH because it is both more stable in plasma and also attenuates formation of the toxic metabolite DHM.

Stability and Metabolite Formation in Dog Plasma. Beagle dog plasma with Na citrate (Innovatine Research, Inc., lot #IBG-NaCitrate-28323) was used in this study. Plasma incubations were carried out in 5 aliquots of 70 μL each (one for each time point), in duplicate. Test compounds (1 μM, final DMSO concentration 1%) were incubated at 37° C. with shaking at 100 rpm. Five time points over 120 minutes were analyzed. The reactions were stopped by adding 400 μL of acetonitrile-methanol mixture (1:1) with subsequent plasma protein sedimentation by centrifugation at 5500 rpm for 5 minutes. Supernatants were analyzed for parent compound remaining and DHM, using a fit-for-purpose liquid chromatography-tandem mass spectrometry (LC-MS/MS) method, with authentic samples of each analyte used for calibration and identification.

### DISCUSSION

It was found, for the first time, that a mitragynine derivative, 3-dehydromitragynine (DHM) is a major metabolite of mitragynine. DHM does not exhibit analgesic activity on its own in mice, but does induce profound signs of toxicity, characterized by ataxia and at sufficiently high doses, death. Therefore, analogs of mitragynine where conversion to DHM is slowed or blocked possess an improved therapeutic ratio between useful therapeutic properties (e.g. analgesia or antidepressant effects) and toxic side effects.

The present invention provides deuterated analogs of mitragynine, 7-OH, and related compounds where the hydrogen (protium) atom at position 3 has been replaced by a deuterium atom. According to the present invention, the greater strength of the deuterium-carbon bond relative to the protium-carbon bond and the resulting kinetic isotope effect attenuates conversion of such 3-deuterated compounds to DHM or their analogous 3-dehydro metabolites compared to the analogous nondeuterated compounds. Similarly, the invention also provides 3-substituted mitragynine derivatives that block conversion to DHM or analogous 3-dehydro compounds. Accordingly, the compounds of the invention provide the therapeutic properties of mitragynine and its analogs with less risk of toxic side effects.

Mitragynine is converted by CYP-mediated metabolism to 7-hydroxymitragynine (7-OH), a metabolite with potent agonist activity at the mu-opioid receptor (MOR) (Kruegel et al. 2016), which is a major contributor to mitragynine's analgesic activity in rodents (Scheme 7). Recent studies have shown that treatment with mitragynine attenuates opioid self-administration in rats (Hemby, S. E., et al. 2018; Yue, K. et al. 2018). At the same time, mitragynine is also converted by other metabolic pathways to 3-dehydromitragynine (DHM), a major metabolite in the brain, which induces toxic effects, such as ataxia, when administered directly to rodents (Scheme 7). DHM is also formed from 7-OH by dehydration and rearrangement under acidic conditions, such as those that occur in the stomach. Accordingly, analogs of mitragynine where formation of DHM (or an analogous metabolite) is blocked, while formation of 7-OH (or an analogous metabolite) is spared, represent compounds with improved separation between analgesia (or ether therapeutic effects) and side effects. Likewise, analogs of 7-OH that attenuate acid-mediated formation of DHM also provide a therapeutic advantage because they limit formation of this toxic metabolite in the acidic environment of the stomach following oral administration.

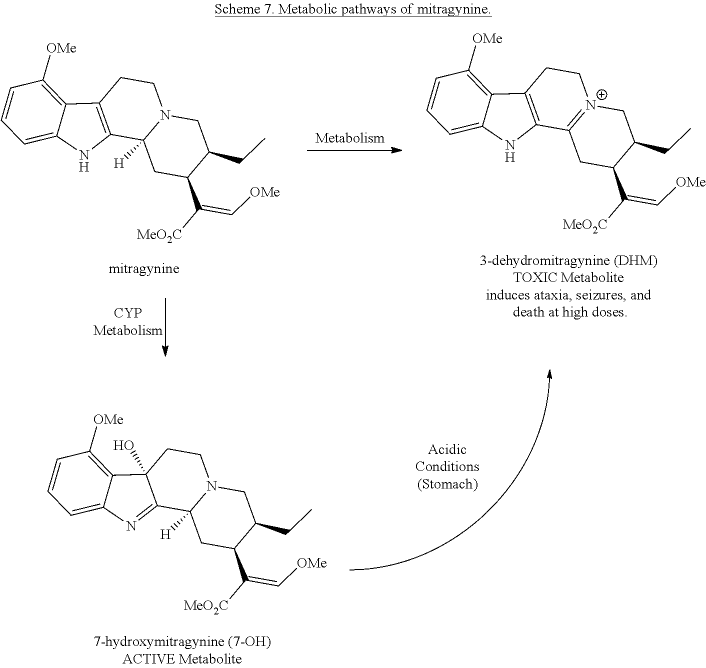 [SMILES: CC[C@@H]1CN2CCc3c([nH]c4cccc(OC)c34)[C@@H]2C[C@@H]1/C(=C\OC)C(=O)OC ; CC[C@@H]1C[N+]2=C(C[C@@H]1/C(=C\OC)C(=O)OC)c1[nH]c3cccc(OC)c3c1CC2 ; CC[C@@H]1CN2CC[C@@]3(O)C(=Nc4cccc(OC)c43)[C@@H]2C[C@@H]1/C(=C\OC)C(=O)OC]

Deuteration of mitragynine at position 3, as in 3-deuteromitragynine (3-DM), attenuates formation of the toxic metabolite DHM via a kinetic isotope effect, while leaving conversion to the active metabolite 3-deutero-7-hydroxymitragynine (3-d-7-OH) unaffected (Scheme 8). Accordingly, 3-DM is a less toxic analog of mitragynine with equivalent analgesic and other therapeutic effects. Further, deuteration of 7-OH, as in 3-d-7-OH, attenuates acid-mediated conversion of this compound to DHM, as occurs in contact with stomach acid (Scheme 8). Accordingly, 3-d-7-OH provides a significant advantage over 7-OH because it permits oral administration with reduced exposure to the toxic metabolite DHM.

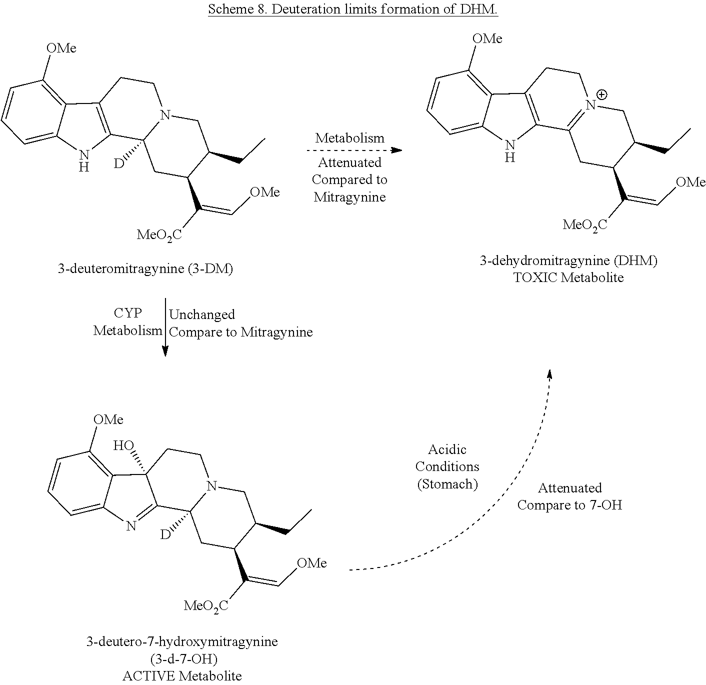 [SMILES: [2H][C@@]12C[C@H](/C(=C\OC)C(=O)OC)[C@H](CC)CN1CCc1c2[nH]c2cccc(OC)c12 ; CC[C@@H]1C[N+]2=C(C[C@@H]1/C(=C\OC)C(=O)OC)c1[nH]c3cccc(OC)c3c1CC2 ; [2H][C@@]12C[C@H](/C(=C\OC)C(=O)OC)[C@H](CC)CN1CC[C@@]1(O)C2=Nc2cccc(OC)c21]

### REFERENCES

Barteselli, A.; Casagrande, M.; Basilico, N.; Parapini, S.; Rusconi, C. M.; Tonelli, M.; Boido, V.; Taramelli, D.; Sparatore, F.; Sparatore, A. Clofazimine analogs with antileishmanial and antiplamodial activity. Bioorg. Med. Chem. 2015, 23, 55-56.
Besson, A. et al. Psychopharmacology 1996, 123, 71-78.
Bodkin, J. A. et al. J. Clin. Psychopharmacol. 1995, 15, 49-57.
Dean, A. J.; Bell, J.; Christie, M. J.; Mattick, R. P. Eur. Psychiatry 2004, 19, 510-513.
Emrich, H. M.; Vogt, P.; Herz, A. Ann. N. Y. Acad. Sci. 1982, 398, 108-112.
Evanno, L.; Ormala, J.; Pihko, P. M. A Highly Enantioselective Access to Tetrahydroisoquinoline and β-Carboline Alkaloids with Simple Noyori-Type Catalysts in Aqueous Media. Chem. Eur. J. 2009, 15, 12963-12967.
Fichna, J.; Janecka, A.; Piestrzeniewicz, M.; Costentin, J.; do Rego, J.-C. Neuropsychopharmacology 2007, 32, 813-821.
Gassaway, M. M. et al. Transl. Psychiatry 2014, 4, e411.
Cerner, R. H. et al. Arch. Gen. Psychiatry 1980, 37, 642-647.
Grinnell, S. G. et al. J. Pharmacol. Exp. Ther. 2014, 350, 710-718.
Grundmann, O. Drug and alcohol dependence 2017, 176, 63-70.
Hemby, S. E.; McIntosh, S.; Leon, F.; Cutler, S. J.; McCurdy, C. R. Addict. Biol. 2018.
Jutkiewicz, E. M. Mol. Interv. 2006, 6, 162-169.
Karp, J. F.; Butters, M. A.; Begley, A. E.; Miller, M. D.; Lenze, E. J.; Blumberger, D. M.; Mulsant, B. H.; Reynolds, C. F. J. Clin. Psychiatry 2014, 75, e785-e793.
Kraepelin, E. Einführung in die psychiatrische Klinik: Zweiunddreissig Vorlesungen; Barth: Leipzig, 1905.
Kramer, M. S. et al. Neuropsychopharmacology 2004, 29, 385-392.
Kruegel, A. C.; Grundmann, O. Neuropharmacology 2018, 134, 108-120.
Kruegel, A. C.; Gassaway, M. M.; Kapoor, A.; Váradi, A.; Majumdar, S.; Filizola, M.; Javitch, J. A.; Sames, D. J. Am. Chem. Soc. 2016, 138, 6754-6764.
Kruegel, A. C. et al. ACS Cent. Sci. 2019, 5, 6, 992-1001.
Largent-Milnes, T.; Yamamoto, T.; Nair, P.; Moulton, J.; Hruby, V.; Lai, J.; Porreca, F.; Vanderah, T. Br. J. Pharmacol. 2010, 161, 986-1001.
Mao, J.; Price, D. D.; Caruso, F. S.; Mayer, D. J. Pain 1996, 67, 361-368.
Moskal, J. R.; Kuo, A. G.; Weiss, C.; Wood, P. L.; Hanson, A. O.; Kelso, S.; Harris, R. B.; Disterhoft, J. F. Neuropharmacology 2005, 49, 1077-1087:
Murrough, J. W. et al. Am. J. Psychiatry 2013, 170, 1134-1142.
Nakagawa, M.; Kawate, T.; Yamazaki, H.; Hino, T. Alkylation of 3,4-dihydro-β-carboline. J. Chem. Soc., Chem. Commun. 1990, 991-992.
Overstreet, D. H.; Naimoli, V. M.; Griebel, G. Pharmacol. Biochem. Behav. 2010, 96, 206-210.
Pain News Network. KRATOM SURVEY—Pain News Network https://www.painnewsnetwork.org/kratom-survey/(accessed Dec. 19, 2018).
Panocka, I. et al. Peptides 2001, 22, 1037-1042.
Pasternak, G. W. Clin. J. Pain 2010, 26 (Supplement 10), S3-S9.
Pasternak, G. W.; Pan, Y.-X. Pharmacol. Rev. 2013, 65, 1257-1317.
Peppin, J. F.; Raffa, R. B. J. Clin. Pharm. Ther. 2015, 40, 155-166.
Piemontesi, C.; Wang, Q.; Zhu, J. Enantioselective Total Synthesis of (−)-Terengganensine A. Angew. Chem. Int. Ed. 2016, 55, 6556-6560
Raffa, R. B. et al. J. Med. Chem. 2013, 56, 4840-4848.
Robinson, J. E.; Fish, E. W.; Krouse, M. C.; Thorsell, A.; Heilig, M.; Malanga, C. J. Psychopharmacology 2012, 220, 215-224.
Rojas-Corrales, M. O.; Gibert-Rahola, J.; Micó, J. A. Life Sci. 1998, 63, PL175-PL180.
Rojas-Corrales, M. O.; Berrocoso, E.; Gibert-Rahola, J.; Micó, J. A. Life Sci. 2002, 72, 143-152.
Saitoh, A.; Kimura, Y.; Suzuki, T.; Kawai, K.; Nagase, H.; Kamei, J. J. Pharmacol. Sci. 2004, 95, 374-380.
Samuels, B. A.; Nautiyal, K. M.; Kruegel, A. C.; Levinstein, M. R.; Magalong, V. M.; Gassaway, M. M.; Grinnell, S. G.; Han, J.; Ansonoff, M. A.; Pintar, J. E.; Javitch, J. A.; Sames, D.; Hen, R. Neuropsychopharmacology 2017, 42, 2052-2063.
Shapira, N. A.; Keck, P. E.; Goldsmith, T. D.; McConville, B. J.; Eis, M.; McElroy, S. L. Depress. Anxiety 1997, 6, 170-173.
Shapira, N. A.; Verduin, M. L.; DeGraw, J. D. J. Clin. Psychiatry 2001, 62, 205-206.
Smith, K. E.; Lawson, T. Drug Alcohol Depend. 2017, 180, 340-348.
Stevenson, G. W. et al. Pharmacol. Biochem. Behay. 2015, 132, 49-55.
Stoll, A. L.; Rueter, S. Am. J. Psychiatry 1999, 156, 2017.
Swogger, M. T.; Hart, E.; Erowid, F.; Erowid, E.; Trabold, N.; Yee, K.; Parkhurst, K. A.; Priddy, B. M.; Walsh, Z. Journal of psychoactive Drugs 2015, 47, 360-367.
Takayama, H. et al. J. Med. Chem. 2002, 45, 1949-1956.
Takayama, H. Chem. Pharm. Bull. 2004, 52, 916-928.
Takayama, H. et al. U.S. Pat. No. 8,648,090 B2, Feb. 11, 2014.
Torregrossa, M. M.; Folk, J. E.; Rice, K. C.; Watson, S. J.; Woods, J. H. Psychopharmacology (Berl). 2005, 183, 31-40.
Trujillo, K. A.; Akil, H. Brain Res. 1994, 633, 178-188.
Vanderah, T. W. Clin. J. Pain. 2010, 26 Suppl, 810-15.
Yue, K.; Kopajtic, T. A.; Katz, J. L. Psychopharmacology 2018, 235, 2823-2829.

1. A composition which comprises a carrier and a compound having the structure: 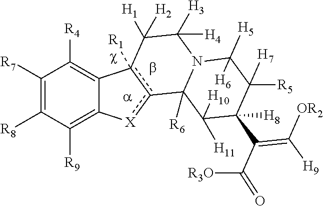
wherein
X is N or NH;
R¹ is —OH, —O-alkyl, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —NO₂, —OH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H—or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl, or a deuterium-enriched —H site;
R₇, R₈ and R₉ are each, independently, —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, —CO₂-(alkyl), —NH(CO)-alkyl, —NH(CO) NH-alkyl, —NH(CO)-aryl, or —NH(CO)NH-aryl;
α is a bond and is absent or present;
β is a bond and is absent or present; and
χ is a bond and is absent or present,
wherein when α is absent, β is present, χ is absent,
X is NH and R₁ is absent, and
wherein when α is present, β is absent, χ is present,
X is N and R₁ is present,
or a pharmaceutically acceptable salt or ester of the compound.

2. The composition of claim 1,
wherein
R₁ is —OH, —O—C(O) (alkyl), or is absent;
R₅ is alkyl or alkenyl;
R₆ is alkyl, aryl, or a deuterium-enriched —H site;
R₇, R₈, and R₉ are each, independently, —H, —F, —Cl, —Br, —I, -alkyl, -alkenyl, -alkynyl, —CN, —CF₃, —NO₂, —OH, —NH₂, —SH, —C(O) NH₂, —C(O)NH(alkyl), —C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H or —CO₂-(alkyl),
or a pharmaceutically acceptable salt or ester of the compound; or
wherein
R₄ is —H, —OH, -alkyl or —O-alkyl;
F₅ is alkyl, alkenyl, alkyl-OH, alkyl-O-alkyl, cycloalkyl, alkyl-cycloalkyl, alkyl-aryl or alkyl-heteroaryl;
R₇, R₈ and R₉ are each, independently, —H, —F, —Cl, —Br, —I, —CN, —CF₃, —NO₂, —OH, —NH₂, —C(O), —NH(CO)-alkyl, —NH(CO)NH-alkyl, —NH(CO)-aryl, —NH(CO)NH-aryl, —O-alkyl, —O-aryl, —O-heteroaryl, alkyl, aryl or heteroaryl,
or a pharmaceutically acceptable salt or ester of the compound.

3. (canceled)

4. The composition of claim 1 wherein the compound has the structure: 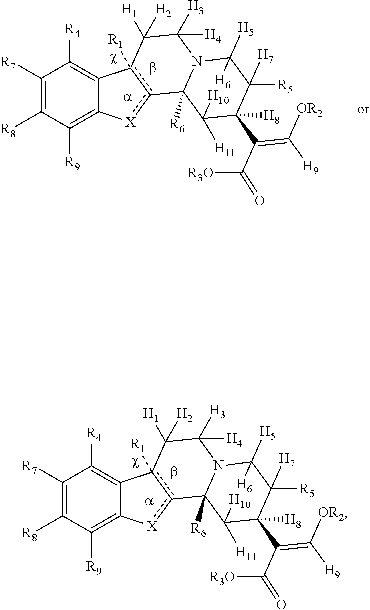
or a pharmaceutically acceptable salt or ester of the compound.

5. The composition of claim 1 wherein the compound has the structure: 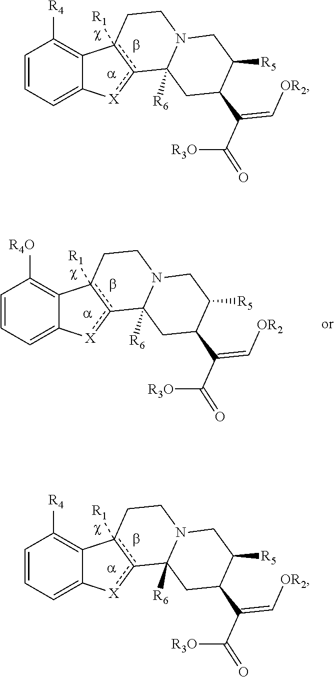
or a pharmaceutically acceptable salt or ester thereof; or 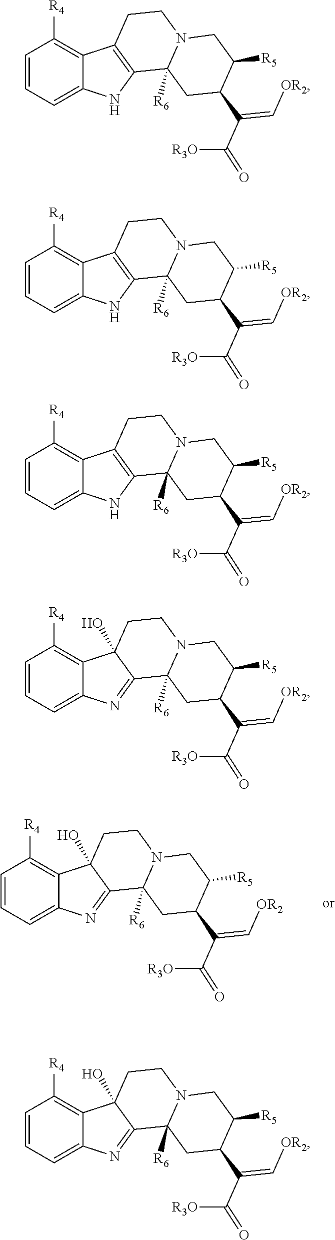
or a pharmaceutically acceptable salt or ester thereof; or 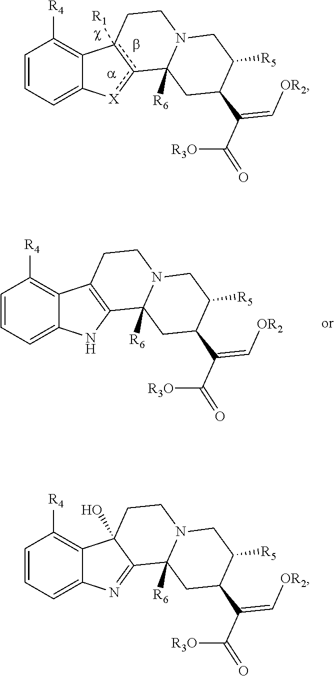
or a pharmaceutically acceptable salt or ester thereof.

6-7. (canceled)

8. The composition of claim 1,
wherein R₂ and R₃ are each methyl; or
wherein R₄ is methoxy; or
wherein R₅ is ethyl or vinyl; or
wherein R₆ is methyl.

9-10. (canceled)

11. The composition of claim 1, wherein one or more of H₁-H₁₁ are deuterium-enriched; or
wherein R₆ is a deuterium-enriched —H site; or
wherein at least one of R₇, R₈ or R₉ is a deuterium-enriched —H site; or
wherein H₁₀ and/or H₁₁ is a deuterium-enriched —H site.

12-15. (canceled)

16. The composition of claim 1 wherein the compound has the structure: 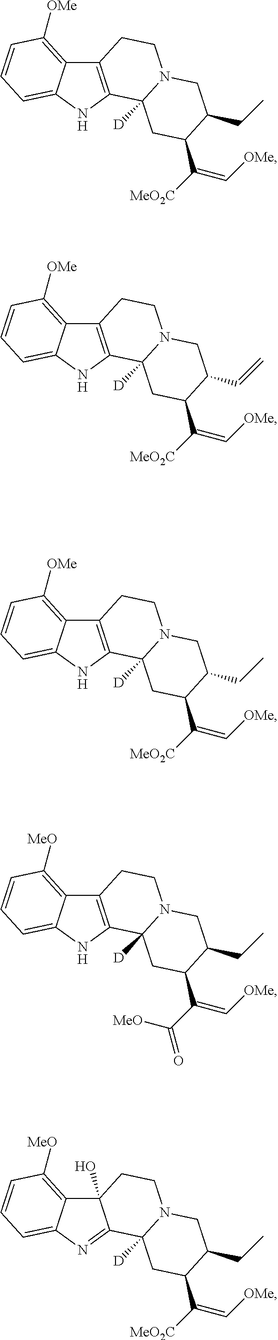 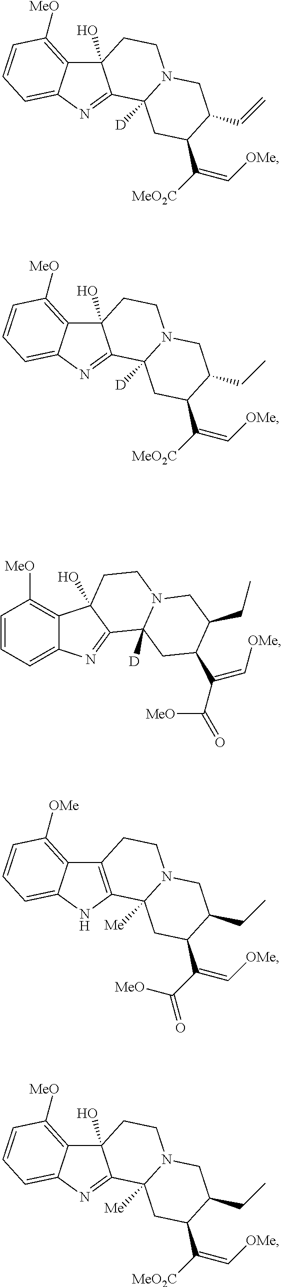 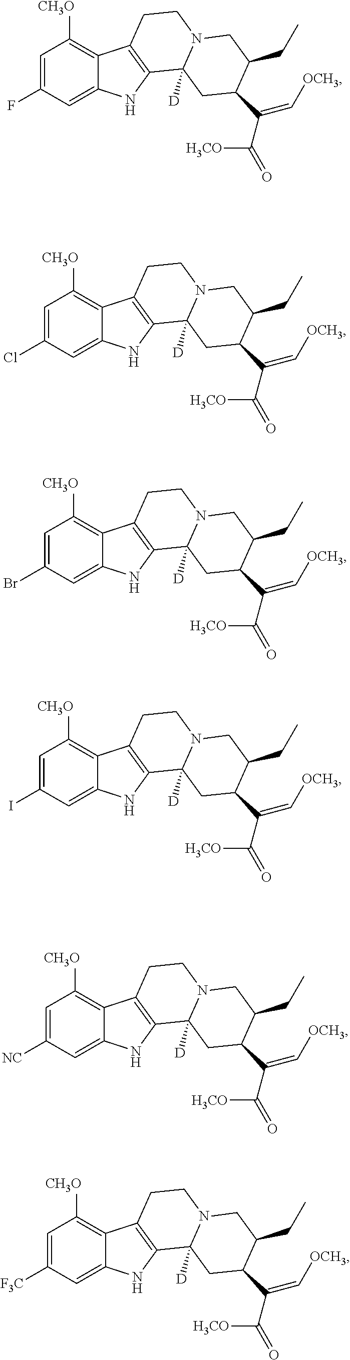 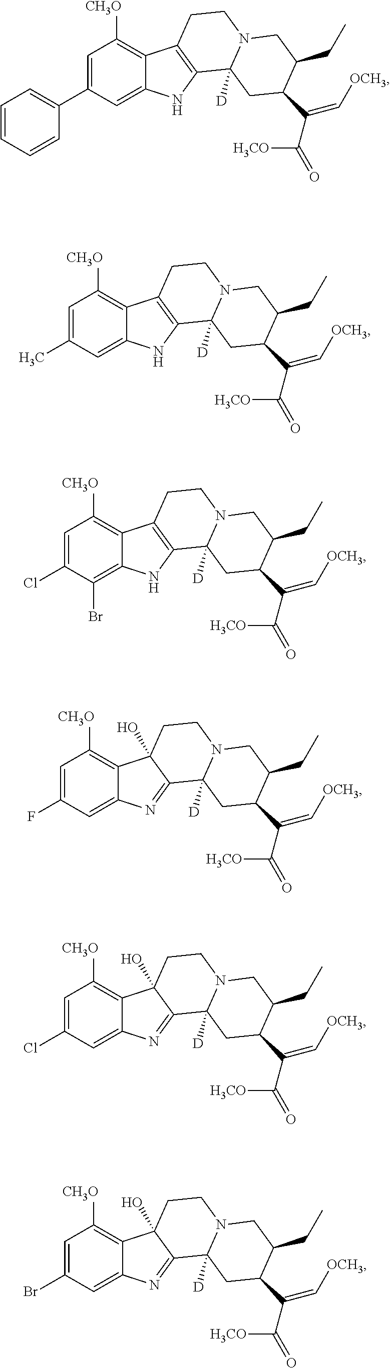 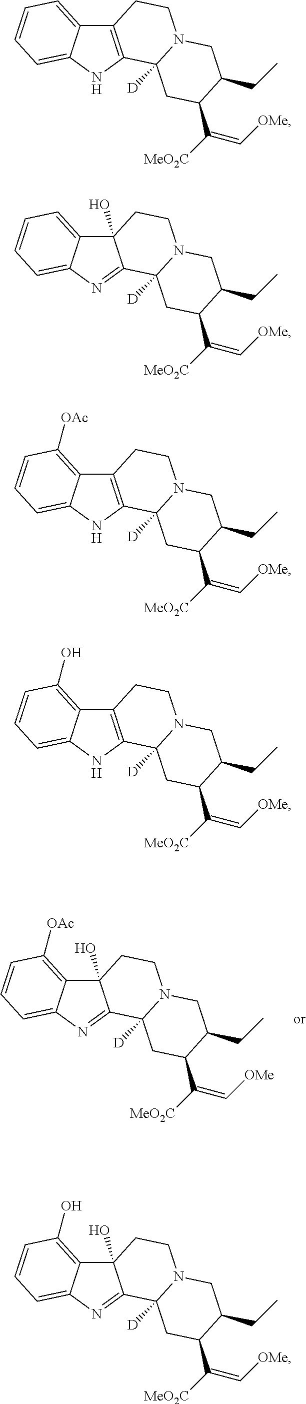
wherein D represents a deuterium-enriched —H site, or a pharmaceutically acceptable salt or ester thereof.

17-18. (canceled)

19. The composition of claim 1, wherein R₆ is a deuterium-enriched —H site and the level of deuterium at the deuterium-enriched —H site of the compound is 0.02% to 100%; or
wherein R₆ is a deuterium-enriched —H site and the level of deuterium at the deuterium-enriched —H site of the compound is 20%-100%, 50%-100%, 70%-100%, 90%-100%, 97%-100%, or 99%-100%; or
wherein R₆ is a deuterium-enriched —H site and the level of deuterium at the deuterium-enriched —H site of the compound is no less than 50%, no less than 70%, no less than 90%, no less than 97% or no less than 99%.

20-21. (canceled)

22. A composition which comprises a mixture of molecules each having the structure: 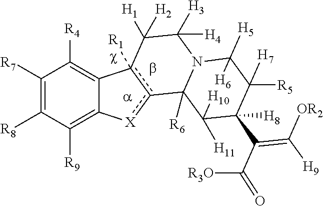
wherein
X is N or NH;
R₁ is —OH, —O-alkyl, —O—C(O) (alkyl), or is absent;
R₂ is —H or -alkyl;
R₃ is —H or -alkyl;
R₄ is —H, —F, —Cl, —Br, —I, -alkyl, -alkenyl, -alkynyl, —CN, —CF₃, —NO₂, —OH, —NH₂, —SH, —C(O)NH₂, —C(O)NH(alkyl), C(O)N(alkyl)₂, —NH(alkyl), —N(alkyl)₂, —O-alkyl, —S-alkyl, —O-aryl, —S-aryl, —O-heteroaryl, —S-heteroaryl, -aryl, -heteroaryl, —O—C(O) (alkyl), —CO₂H, or —CO₂-(alkyl);
R₅ is alkyl, alkenyl, alkyl-OH, alkyl-O-alkyl, cycloalkyl, alkyl-cycloalkyl, alkyl-aryl or alkyl-heteroaryl;
R₆ is alkyl, aryl or a deuterium-enriched —H site;
R₇, R₈ and R₉ are each, independent —H, —F, —Cl, —Br, —I, -alkyl, -alkenyl, -alkynyl, —CN, —CF₃, —NO₂, —OH, —NH₂, —SH, —C(O) NH₂, —C(O) NH (alkyl), —C(O)N(alkyl)₂, —NH(alkyl), —N(alkyl)₂, —O-alkyl, —S-alkyl, —O-aryl, —S-aryl, —O-heteroaryl, —S— heteroaryl, -aryl, -heteroaryl, —O—C(O) (alkyl), —CO₂H, —CO₂-(alkyl), —NH(CO)-alkyl, —NH(CO) NH— alkyl, —NH(CO)-aryl, or —NH(CO)NH-aryl;
α is a bond and is absent or present;
β is a bond and is absent or present; and
χ is a bond and is absent or present,
wherein when α is absent, β is present, χ is absent,
X is NH and R₁ is absent, and
wherein when α is present, β is absent, χ is present,
X is N and R₁ is present,
or a pharmaceutically acceptable salt or ester of the compound,
wherein when R₆ is a deuterium-enriched —H site, the proportion of molecules having deuterium at the —R₆ position is substantially greater than 0.0156% of molecules in the composition.

23. The composition claim 22, wherein the proportion of molecules having deuterium at the —R₆ position is substantially greater than 90% of molecules in the composition.

24. The composition of claim 22 wherein the compound having deuterium at the —R₆ deuterium-enriched —H site is 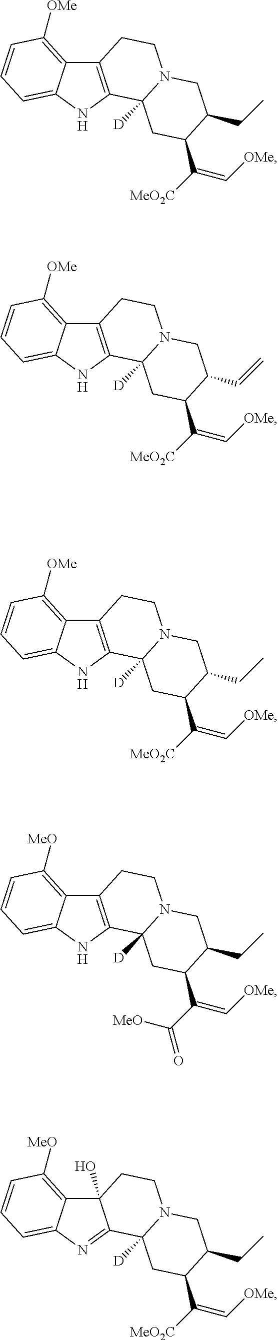 [SMILES: [2H][C@@]12C[C@H](/C(=C\OC)C(=O)OC)[C@H](CC)CN1CCc1c2[nH]c2cccc(OC)c12 ; [2H][C@@]12C[C@H](/C(=C\OC)C(=O)OC)[C@@H](C=C)CN1CCc1c2[nH]c2cccc(OC)c12 ; [2H][C@@]12C[C@H](/C(=C\OC)C(=O)OC)[C@@H](CC)CN1CCc1c2[nH]c2cccc(OC)c12 ; [2H][C@]12C[C@H](/C(=C\OC)C(=O)OC)[C@H](CC)CN1CCc1c2[nH]c2cccc(OC)c12 ; [2H][C@@]12C[C@H](/C(=C\OC)C(=O)OC)[C@H](CC)CN1CC[C@@]1(O)C2=Nc2cccc(OC)c21] 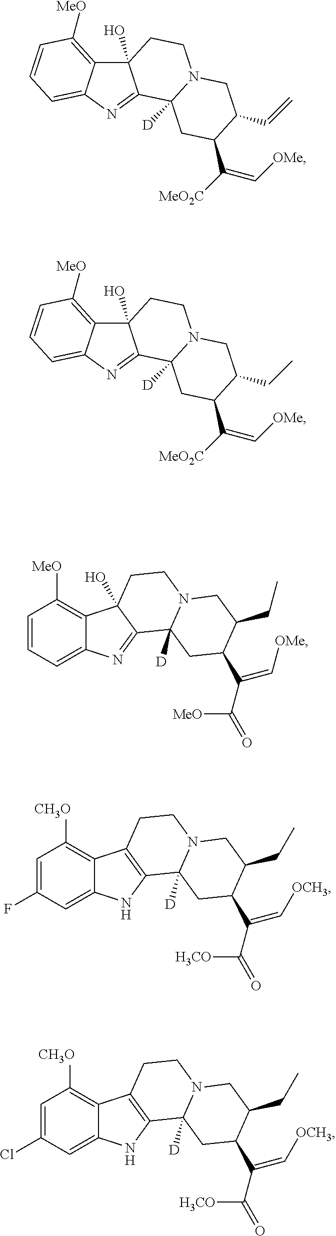 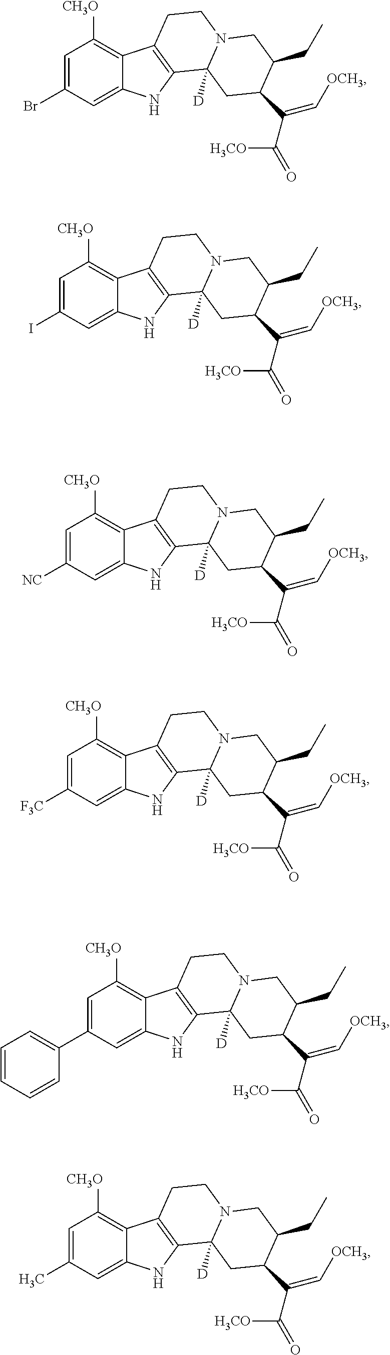 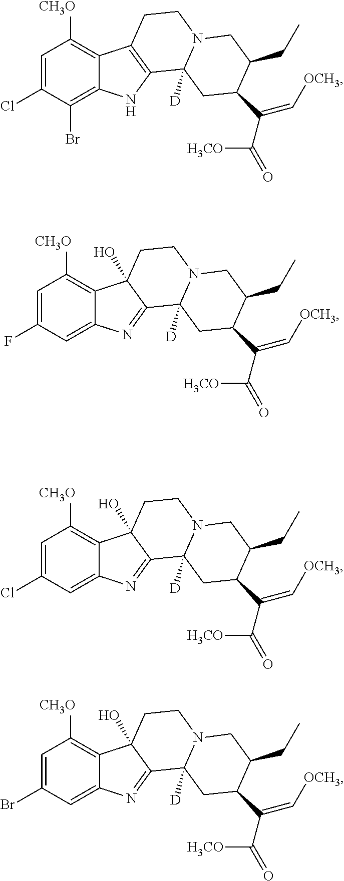 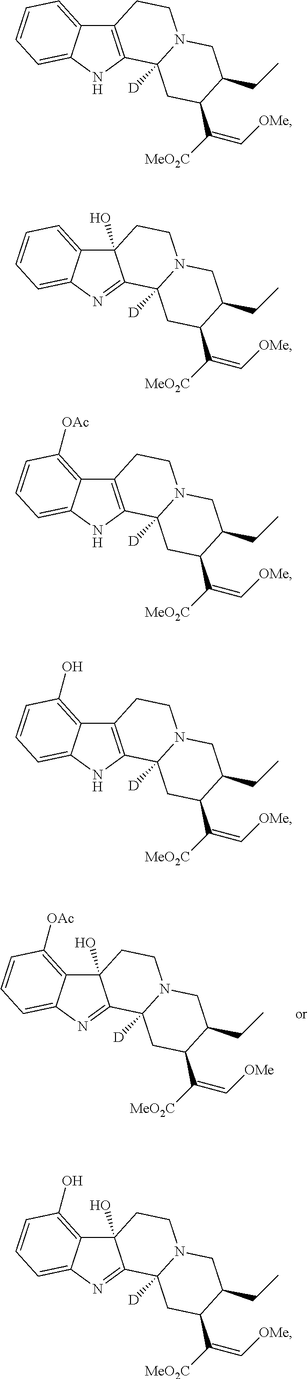 [SMILES: [2H][C@@]12C[C@H](/C(=C\OC)C(=O)OC)[C@H](CC)CN1CCc1c2[nH]c2ccccc12 ; [2H][C@@]12C[C@H](/C(=C\OC)C(=O)OC)[C@H](CC)CN1CC[C@@]1(O)C2=Nc2ccccc21 ; [2H][C@@]12C[C@H](/C(=C\OC)C(=O)OC)[C@H](CC)CN1CCc1c2[nH]c2cccc(OC(C)=O)c12 ; [2H][C@@]12C[C@H](/C(=C\OC)C(=O)OC)[C@H](CC)CN1CCc1c2[nH]c2cccc(O)c12 ; [2H][C@@]12C[C@H](/C(=C\OC)C(=O)OC)[C@H](CC)CN1CC[C@@]1(O)C2=Nc2cccc(OC(C)=O)c21 ; [2H][C@@]12C[C@H](/C(=C\OC)C(=O)OC)[C@H](CC)CN1CC[C@@]1(O)C2=Nc2cccc(O)c21]
or a pharmaceutically acceptable salt or ester thereof.

25-26. (canceled)

27. The composition of claim 1, further comprising a carrier.

28. The composition of claim 1, wherein the carrier is a pharmaceutically acceptable carrier.

29. The composition of claim 28, further comprising an NMDA receptor antagonist, an NMDA receptor partial agonist, a neurokinin 1 receptor antagonist, a neurokinin 2 receptor antagonist, a neurokinin 3 receptor antagonist, a DOR agonist, naloxone, methylnaltrexone, a selective serotonin reuptake inhibitor or a serotonin-norepinephrine reuptake inhibitor.

30. The composition of claim 29, wherein the NMDA receptor antagonist is ibogaine or noribogaine.

31. A method of activating a mu-opioid receptor comprising contacting the mu-opioid receptor with the composition of claim 1, or
of antagonizing a delta-opioid receptor and/or a kappa-opioid receptor comprising contacting the delta-opioid receptor and/or the kappa-opioid receptor with the composition of claim 1.

32. (canceled)

33. A method of treating a subject afflicted with pain, a depressive disorder, a mood disorder, an anxiety disorder, borderline personality disorder, substance use disorder, opioid use disorder or opioid withdrawal symptoms comprising administering an effective amount of the composition of claim 1 to the subject so as to thereby treat the subject afflicted with pain, the depressive disorder, mood disorder, anxiety disorder, borderline personality disorder, a substance use disorder, opioid use disorder or opioid withdrawal symptoms.

34. A method of treating a subject afflicted with pain comprising administering to the subject an effective amount of an NMDA receptor antagonist, an NMDA receptor partial agonist, a neurokinin 1 receptor antagonist, or a delta-opioid receptor agonist and an effective amount of the composition of claim 1 so as to thereby treat the subject afflicted with pain, or
of treating a subject afflicted with a depressive disorder or mood disorder comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist, or a delta-opioid receptor agonist and an effective amount of the composition of claim 1 so as to thereby treat the subject afflicted with the depressive disorder or mood disorder, or
of treating a subject afflicted with an anxiety disorder comprising administering to the subject an effective amount of an NMDA receptor antagonist, an NMDA receptor partial agonist, a neurokinin 1 receptor antagonist, a neurokinin 2 receptor antagonist, a neurokinin 3 receptor antagonist, or a delta-opioid receptor agonist and an effective amount of the composition of claim 1 so as to thereby treat the subject afflicted with the anxiety disorder, or
of treating a subject afflicted with borderline personality disorder comprising administering to the subject an effective amount: of an NMDA receptor antagonist, an NMDA receptor partial agonist, a neurokinin 1 receptor antagonist, or a CUR agonist and an effective amount of the composition of claim 1 so as to thereby treat the subject afflicted with borderline personality disorder, or
of treating a subject afflicted with opioid use disorder or opioid withdrawal symptoms comprising administering to the subject an effective amount of an NMDA receptor antagonist, an NMDA receptor partial agonist, or a neurokinin 1 receptor antagonist and an effective amount of the composition of claim 1 so as to thereby treat the subject: afflicted with the opioid use disorder or opioid withdrawal symptoms, or
of treating a subject afflicted with opioid use disorder or opioid withdrawal symptoms comprising administering to the subject an effective amount of naloxone or methylnaltrexone and an effective amount of the composition of claim 1 so as to thereby treat the subject afflicted with the opioid use disorder or opioid withdrawal symptoms, or
of treating a subject afflicted with pain, a depressive disorder, a mood disorder, an anxiety disorder, or borderline personality disorder, comprising administering to the subject an effective amount of naloxone or methylnaltrexone and an effective amount of the composition of claim 1 so as to thereby treat the subject afflicted with pain, the depressive disorder, the mood disorder, the anxiety disorder, or borderline personality disorder, or
of treating a subject afflicted with a depressive disorder, a mood disorder, an anxiety disorder, or borderline personality disorder, comprising administering to the subject an effective amount of a selective serotonin reuptake inhibitor or a serotonin-norepinephrine reuptake inhibitor and an effective amount of the composition of claim 1 so as to thereby treat the subject afflicted with the depressive disorder, the mood disorder, the anxiety disorder, or borderline personality disorder.

35. A process for producing a composition comprising a compound having the structure: 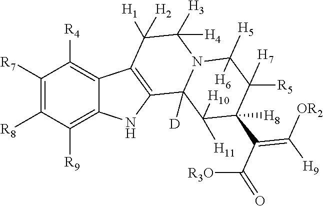
wherein C represents a hydrogen site which is deuterium-enriched, comprising
(i) reacting the compound having the following structure: 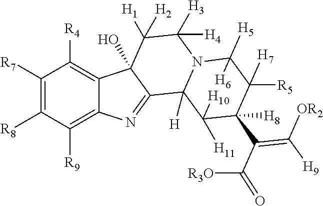
with an acid in a first suitable solvent so as to thereby produce the compound having the following structure: 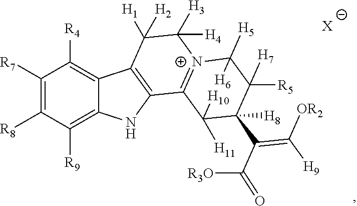
wherein X⁻ is a suitable counter ion; and
(ii) reacting the product of step (i) with NaBD₄ in a second suitable solvent under conditions sufficient to thereby produce the compound; or
producing a composition comprising a compound having the structure: 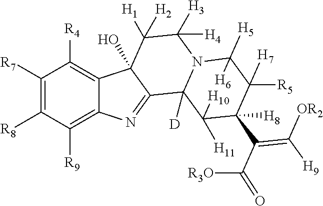
wherein D represents a deuterium-enriched site,
comprising
(i) reacting the compound having the following structure: 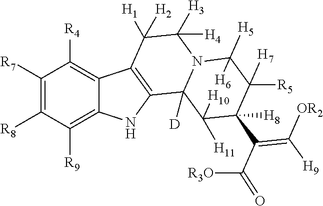
with an oxidizing agent in a suitable solvent under conditions sufficient to thereby produce the compound.

36. (canceled)

37. A method for systemic in vivo delivery of a first composition of claim 1 which comprises a first carrier and a first compound having the structure: 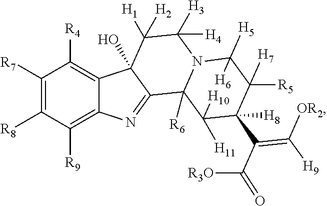
to a subject, the method comprising administering to the subject a second composition of claim 1 which comprises a second carrier and a second compound having the structure: 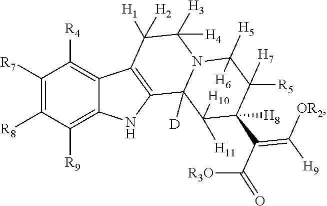
so as to thereby deliver the first compound to the subject.
